(19) United States
(12) Patent Application Publication
(10) Pub. No.: US 2022/0056469 A1
(43) Pub. Date: Feb. 24, 2022

(54) INSECTICIDAL PROTEINS AND METHODS FOR THEIR USE

(71) Applicant: PIONEER HI-BRED INTERNATIONAL, INC., JOHNSTON, IA (US)

(72) Inventors: DANIEL J ALTIER, GRANGER, IA (US); JENNIFER KARA BARRY, AMES, IA (US); CHRISTIAN BARTHOLOMAY, MADISON, WI (US); HUA DONG, JOHNSTON, IA (US); RYAN MICHAEL GERBER, APEX, NC (US); JACOB GILLIAM, NORWALK, IA (US); STEVEN D GRUVER, PACIFICA, CA (US); LU LIU, PALO ALTO, CA (US); JARRED KENNETH ORAL, JOHNSTON, IA (US); JESSICA O'REAR, REDWOOD CITY, CA (US); UTE SCHELLENBERGER, PALO ALTO, CA (US); ERIC SCHEPERS, PORT DEPOSIT, MD (US); DANIEL JAMES THORPE, JOHNSTON, IA (US); THOMAS CHAD WOLFE, DES MOINES, IA (US); WEIPING XIE, EAST PALO ALTO, CA (US); NASSER YALPANI, KELOWNA (CA); YOU YOU, FREMONT, CA (US); GENHAI ZHU, SAN JOSE, CA (US)

(73) Assignee: PIONEER HI-BRED INTERNATIONAL, INC., JOHNSTON, IA (US)

(21) Appl. No.: 17/274,888

(22) PCT Filed: Sep. 10, 2019
(86) PCT No.: PCT/US19/50434

§ 371 Date: Mar. 10, 2021

Related U.S. Application Data
(60) Provisional application No. 62/729,759, filed on Sep. 11, 2018.

(51) Int. Cl. C12N 15/82; C07K 14/21; A01N 63/50; C07K 14/195

(52) U.S. Cl. CPC C12N 15/8286; C07K 14/195; A01N 63/50; C07K 14/21

(57) ABSTRACT

Compositions and methods for controlling pests are provided. The methods involve transforming organisms with a nucleic acid sequence encoding an insecticidal protein. In particular, the nucleic acid sequences are useful for preparing plants and microorganisms that possess insecticidal activity. Thus, transformed bacteria, plants, plant cells, plant tissues and seeds are provided. Compositions are insecticidal nucleic acids and proteins of bacterial species. The sequences find use in the construction of expression vectors for subsequent transformation into organisms of interest including plants, as probes for the isolation of other homologous (or partially homologous) genes. The pesticidal proteins find use in controlling, inhibiting growth or killing Lepidopteran, Coleopteran, Dipteran, fungal, Hemipteran and nematode pest populations and for producing compositions with insecticidal activity.

### REFERENCE TO SEQUENCE LISTING SUBMITTED ELECTRONICALLY

The official copy of the sequence listing is submitted electronically via EFS-Web as an ASCII formatted sequence listing with a file named “6456WOPCT_SequenceListing” created on Sep. 10, 2019, and having a size of 1,557 kilobytes and is filed concurrently with the specification. The sequence listing contained in this ASCII formatted document is part of the specification and is herein incorporated by reference in its entirety.

### FIELD

This disclosure relates to the field of molecular biology. Provided are novel genes encoding pesticidal proteins. These pesticidal proteins and the nucleic acid sequences encoding them are useful in preparing pesticidal formulations and in the production of transgenic pest-resistant plants.

### BACKGROUND

Biological control of insect pests of agricultural significance using a microbial agent, such as fungi, bacteria or another species of insect affords an environmentally friendly and commercially attractive alternative to synthetic chemical pesticides. The use of biopesticides presents a lower risk of pollution and environmental hazards and biopesticides provide greater target specificity than is characteristic of traditional broad-spectrum chemical insecticides. In addition, biopesticides often cost less to produce and thus improve economic yield for a wide variety of crops.

Certain species of microorganisms of the genus Bacillus are known to possess pesticidal activity against a range of insect pests including Lepidoptera, Diptera, Coleoptera, Hemiptera and others. Bacillus thuringiensis (Bt) and Bacillus popilliae are among the most successful biocontrol agents discovered to date. Insect pathogenicity has also been attributed to strains of B. larvae, B. lentimorbus, B. sphaericus and B. cereus. Microbial insecticides, particularly those obtained from Bacillus strains, have played an important role in agriculture as alternatives to chemical pest control.

Crop plants have been developed with enhanced insect resistance by genetically engineering crop plants to produce pesticidal proteins from Bacillus. For example, corn and cotton plants have been genetically engineered to produce pesticidal proteins isolated from strains of Bacillus thuringiensis. These genetically engineered crops are now widely used in agriculture and have provided the farmer with an environmentally friendly alternative to traditional insect-control methods. While they have proven to be very successful commercially, these genetically engineered, insect-resistant crop plants provide resistance to only a narrow range of the economically important insect pests. In some cases, insects can develop resistance to different insecticidal compounds, which raises the need to identify alternative biological control agents for pest control.

Accordingly, there remains a need for new pesticidal proteins with different ranges of insecticidal activity against insect pests, e.g., insecticidal proteins which are active against a variety of insects in the order Lepidoptera and the order Coleoptera including but not limited to insect pests that have developed resistance to existing insecticides.

### SUMMARY

In one aspect, compositions and methods for conferring pesticidal activity to bacteria, plants, plant cells, tissues and seeds are provided. Compositions include nucleic acid molecules encoding sequences for pesticidal and insecticidal polypeptides, vectors comprising those nucleic acid molecules, and host cells comprising the vectors. Compositions also include the pesticidal polypeptide sequences and antibodies to those polypeptides. Compositions also comprise transformed bacteria, plants, plant cells, tissues and seeds.

In another aspect, isolated or recombinant nucleic acid molecules are provided encoding IPD092-1 polypeptides, IPD092-2 polypeptides, IPD095-1 polypeptides, IPD095-2 polypeptides, IPD097 polypeptides, IPD099-1 polypeptides, IPD099-2 polypeptides, IPD099-3 polypeptides, IPD100-1 polypeptides, IPD100-2 polypeptides, IPD105 polypeptides, IPD106-1 polypeptides, IPD106-2 polypeptides, IPD107 polypeptides, IPD111 polypeptides, and IPD112 polypeptides including amino acid substitutions, deletions, insertions, and insecticidally active portions thereof. Nucleic acid sequences that are complementary to a nucleic acid sequence of the embodiments or that hybridize to a sequence of the embodiments are also encompassed. The nucleic acid sequences can be used in DNA constructs or expression cassettes for transformation and expression in organisms, including microorganisms and plants. The nucleotide or amino acid sequences may be synthetic sequences that have been designed for expression in an organism including, but not limited to, a microorganism or a plant.

In another aspect, IPD092-1 polypeptides, IPD092-2 polypeptides, IPD095-1 polypeptides, IPD095-2 polypeptides, IPD097 polypeptides, IPD099-1 polypeptides, IPD099-2 polypeptides, IPD099-3 polypeptides, IPD100-1 polypeptides, IPD100-2 polypeptides, IPD105 polypeptides, IPD106-1 polypeptides, IPD106-2 polypeptides, IPD107 polypeptides, IPD111 polypeptides, and IPD112 polypeptides are encompassed. Also provided are isolated or recombinant IPD092-1 polypeptides, IPD092-2 polypeptides, IPD095-1 polypeptides, IPD095-2 polypeptides, IPD097 polypeptides, IPD099-1 polypeptides, IPD099-2 polypeptides, IPD099-3 polypeptides, IPD100-1 polypeptides, IPD100-2 polypeptides, IPD105 polypeptides, IPD106-1 polypeptides, IPD106-2 polypeptides, IPD107 polypeptides, IPD111 polypeptides, and IPD112 polypeptides, as well as amino acid substitutions, deletions, insertions, insecticidally active portions thereof and combinations thereof are provided.

In another aspect, methods are provided for producing the polypeptides and for using those polypeptides for controlling or killing a Lepidopteran, Coleopteran, nematode, fungi, and/or Dipteran pests. The transgenic plants of the embodiments express one or more of the pesticidal sequences disclosed herein. In various embodiments, the transgenic plant further comprises one or more additional genes for insect resistance, for example, one or more additional genes for controlling Coleopteran, Lepidopteran, Hemipteran or nematode pests. It will be understood by one of skill in the art that the transgenic plant may comprise any gene imparting an agronomic trait of interest.

In another aspect, methods for detecting the nucleic acids and polypeptides of the embodiments in a sample are also included. A kit for detecting the presence of a polypeptide of the disclosure or detecting the presence of a polynucleotide encoding a polypeptide of the disclosure in a sample is provided. The kit may be provided along with all reagents and control samples necessary for carrying out a method for detecting the intended agent, as well as instructions for use.

In another aspect, the compositions and methods of the embodiments are useful to produce organisms with enhanced pest resistance or tolerance. These organisms and compositions comprising the organisms are desirable for agricultural purposes. The compositions of the embodiments are also useful for generating altered or improved proteins that have pesticidal activity or for detecting the presence of the polypeptides of the disclosure.

### BRIEF DESCRIPTION OF THE FIGURES

FIG. 1A-1B shows an amino acid sequence alignment, using the ALIGNX® module of the Vector NTI® suite, of IPD092-1Aa polypeptide (SEQ ID NO: 1), IPD092-1Ab polypeptide (SEQ ID NO: 3), IPD092-1Ba polypeptide (SEQ ID NO: 5), IPD092-1Bb polypeptide (SEQ ID NO: 7), IPD092-1Ca polypeptide (SEQ ID NO: 9), IPD092-1Cb polypeptide (SEQ ID NO: 11), IPD092-1Da polypeptide (SEQ ID NO: 13), IPD092-1Db polypeptide (SEQ ID NO: 15), IPD092-1Ea polypeptide (SEQ ID NO: 17), IPD092-1Eb polypeptide (SEQ ID NO: 19), IPD092-1Fa polypeptide (SEQ ID NO: 22), IPD092-1Fb polypeptide (SEQ ID NO: 24), and IPD092-1Fc polypeptide (SEQ ID NO: 26). The amino acid sequence diversity between the amino acid sequences is highlighted.

FIG. 2A-2B shows an amino acid sequence alignment, using the ALIGNX® module of the Vector NTI® suite, of IPD092-2Aa polypeptide (SEQ ID NO: 2), IPD092-2Ab polypeptide (SEQ ID NO: 4), IPD092-2Ba polypeptide (SEQ ID NO: 6), IPD092-2Bb polypeptide (SEQ ID NO: 8), IPD092-2Ca polypeptide (SEQ ID NO: 10), IPD092-2Cb polypeptide (SEQ ID NO: 12), IPD092-2Da polypeptide (SEQ ID NO:14), IPD092-2Db polypeptide (SEQ ID NO: 16), IPD092-2Ea polypeptide (SEQ ID NO: 18), IPD092-2Eb polypeptide (SEQ ID NO: 20), IPD092-2Ec polypeptide (SEQ ID NO: 21), IPD092-2Fa polypeptide (SEQ ID NO: 23), IPD092Fb-2 polypeptide (SEQ ID NO: 25. The amino acid sequence diversity between the amino acid sequences is highlighted.

### DETAILED DESCRIPTION

It is to be understood that this disclosure is not limited to the methodologies, protocols, cell lines, genera, and reagents described, as such may vary. It is also to be understood that the terminology used herein is for the purpose of describing embodiments only, and is not intended to limit the scope of the present disclosure.

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof. All technical and scientific terms used herein have the same meaning as commonly understood to one of ordinary skill in the art to which this disclosure belongs unless clearly indicated otherwise.

The present disclosure is drawn to compositions and methods for controlling pests. The methods involve transforming organisms with nucleic acid sequences encoding IPD092-1 polypeptides, IPD092-2 polypeptides, IPD095-1 polypeptides, IPD095-2 polypeptides, IPD097 polypeptides, IPD099-1 polypeptides, IPD099-2 polypeptides, IPD099-3 polypeptides, IPD100-1 polypeptides, IPD100-2 polypeptides, IPD105 polypeptides, IPD106-1 polypeptides, IPD106-2 polypeptides, IPD107 polypeptides, IPD111 polypeptides, and IPD112 polypeptides. The nucleic acid sequences of the embodiments are useful for preparing plants and microorganisms that possess pesticidal activity. Thus, transformed bacteria, plants, plant cells, plant tissues and seeds are provided. The compositions include pesticidal nucleic acids and proteins of bacterial species. The nucleic acid sequences find use in the construction of expression vectors for subsequent transformation into organisms of interest, as probes for the isolation of other homologous (or partially homologous) genes, and for the generation of altered IPD092-1 polypeptides, IPD092-2 polypeptides, IPD095-1 polypeptides, IPD095-2 polypeptides, IPD097 polypeptides, IPD099-1 polypeptides, IPD099-2 polypeptides, IPD099-3 polypeptides, IPD100-1 polypeptides, IPD100-2 polypeptides, IPD105 polypeptides, IPD106-1 polypeptides, IPD106-2 polypeptides, IPD107 polypeptides, IPD111 polypeptides, and IPD112 polypeptides by methods such as site directed mutagenesis, domain swapping or DNA shuffling. The IPD092-1 polypeptides, IPD092-2 polypeptides, IPD095-1 polypeptides, IPD095-2 polypeptides, IPD097 polypeptides, IPD099-1 polypeptides, IPD099-2 polypeptides, IPD099-3 polypeptides, IPD100-1 polypeptides, IPD100-2 polypeptides, IPD105 polypeptides, IPD106-1 polypeptides, IPD106-2 polypeptides, IPD107 polypeptides, IPD111 polypeptides, and IPD112 polypeptides find use in controlling or killing Lepidopteran, Coleopteran, Dipteran, fungal, Hemipteran and nematode pest populations and for producing compositions with pesticidal activity. Insect pests of interest include, but are not limited to, Lepidoptera species including but not limited to: Corn Earworm, (CEVV) (Helicoverpa zea), European Corn Borer (ECB) (Ostrinia nubialis), diamond-back moth, e.g., Helicoverpa zea Boddie; soybean looper, e.g., Pseudoplusia includens Walker; and velvet bean caterpillar e.g., Anticarsia gemmatalis Hübner and Coleoptera species including but not limited to Western corn rootworm (Diabrotica virgifera)—WCRW, Southern corn rootworm (Diabrotica undecimpunctata howardi)—SCRW, and Northern corn rootworm (Diabrotica barberi)—NCRW.

By “pesticidal toxin” or “pesticidal protein” or “insecticidal protein” is used herein to refer to a toxin that has toxic activity against one or more pests, including, but not limited to, members of the Lepidoptera, Diptera, Hemiptera and Coleoptera orders or the Nematoda phylum or a protein that has homology to such a protein. Pesticidal proteins have been isolated from organisms including, for example, Bacillus sp., Pseudomonas sp., Photorhabdus sp., Xenorhabdus sp., Clostridium bifermentans and Paenibacillus popilliae.

In some embodiments, the polypeptide of the disclosure includes an amino acid sequence deduced from the full-length nucleic acid sequence disclosed herein and amino acid sequences that are shorter than the full-length sequences, either due to the use of an alternate downstream start site or due to processing that produces a shorter protein having pesticidal activity. Processing may occur in the organism the protein is expressed in or in the pest after ingestion of the protein.

Thus, provided herein are novel isolated or recombinant nucleic acid sequences that confer pesticidal activity. Also provided are the amino acid sequences of polypeptides of the disclosure. The protein resulting from translation of these genes encoding the polypeptides of the disclosure allows cells to control or kill pests that ingest it.

### IPD092-1 and IPD092-2 Proteins and Variants and Fragments Thereof

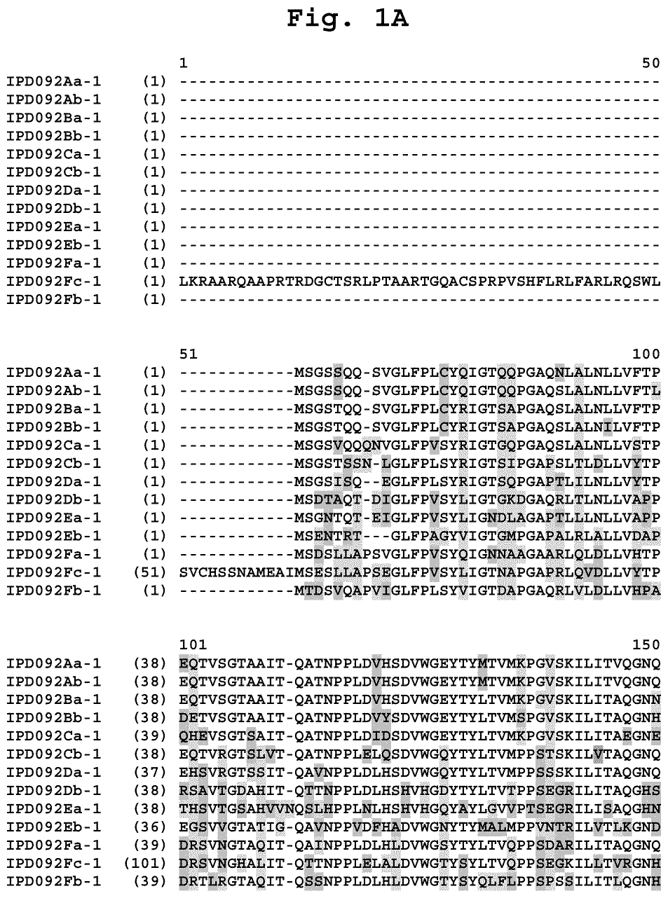

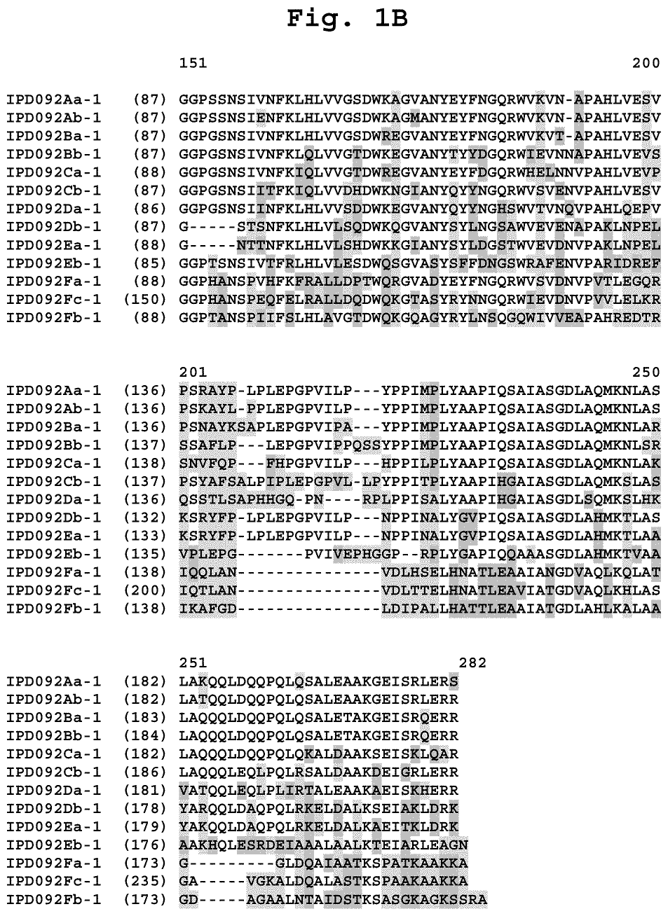

IPD092-1 and IPD092-2 polypeptides are encompassed by the disclosure. “IPD092-1 polypeptide”, and “IPD092-1 protein” as used herein interchangeably refers to a polypeptide having insecticidal activity, in combination with a IPD092-2 polypeptide, against one or more insect pests of the Lepidoptera and/or Coleoptera orders including but not limited to Western corn rootworm (WCRW), and is sufficiently homologous to the IPD092-1 polypeptide of SEQ ID NO: 1. A variety of IPD092-1 polypeptide homologs are contemplated. Sources of IPD092-1 polypeptide homologs or related proteins include bacterial species selected from but not limited to Pseudomonas species, Chromobacterium species, Burkholderia species, and Woodsholea species. Alignment of the amino acid sequences of IPD092-1 polypeptide homologs (for example—FIG. 1A-1B), allows for the identification of residues that are highly conserved amongst the natural homologs of this family.

“Sufficiently homologous” is used herein to refer to an amino acid sequence that has at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence homology compared to a reference sequence using one of the alignment programs described herein using standard parameters. In some embodiments, the sequence homology is against the full-length sequence of an IPD092-1 polypeptide. In some embodiments, the IPD092-1 polypeptide has at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 22, SEQ ID NO: 24 or SEQ ID NO: 26. The term “about” when used herein in context with percent sequence identity means +/−0.5%. One of skill in the art will recognize that these values can be appropriately adjusted to determine corresponding homology of proteins considering amino acid similarity and the like. In some embodiments, the sequence identity is calculated using ClustalW algorithm in the ALIGNX® module of the Vector NTI® Program Suite (Invitrogen Corporation, Carlsbad, Calif.) with all default parameters. In some embodiments, the sequence identity is across the entire length of polypeptide calculated using ClustalW algorithm in the ALIGNX® module of the Vector NTI® Program Suite (Invitrogen Corporation, Carlsbad, Calif.) with all default parameters.

In some embodiments an IPD092-1 polypeptide comprises an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 22, SEQ ID NO: 24 or SEQ ID NO: 26.

In some embodiments, the sequence identity is across the entire length of the polypeptide calculated using ClustalW algorithm in the ALIGNX® module of the Vector NTI® Program Suite (Invitrogen Corporation, Carlsbad, Calif.) with all default parameters.

In some embodiments an IPD092-1 polypeptide comprises an amino acid sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 22, SEQ ID NO: 24 or SEQ ID NO: 26 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70,71, 72, 73, 74, 75, 76, 77, 78, 79, 80, 81, 82, 83, 84, 85, 86, 87, 88, 89, 90, 91, 92, 93, 94, 95 or more amino acid substitutions compared to the native amino acid at the corresponding position of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 22, SEQ ID NO: 24 or SEQ ID NO: 26.

In some embodiments, the IPD092-1 polypeptide comprises the amino acid sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 22, SEQ ID NO: 24 or SEQ ID NO: 26.

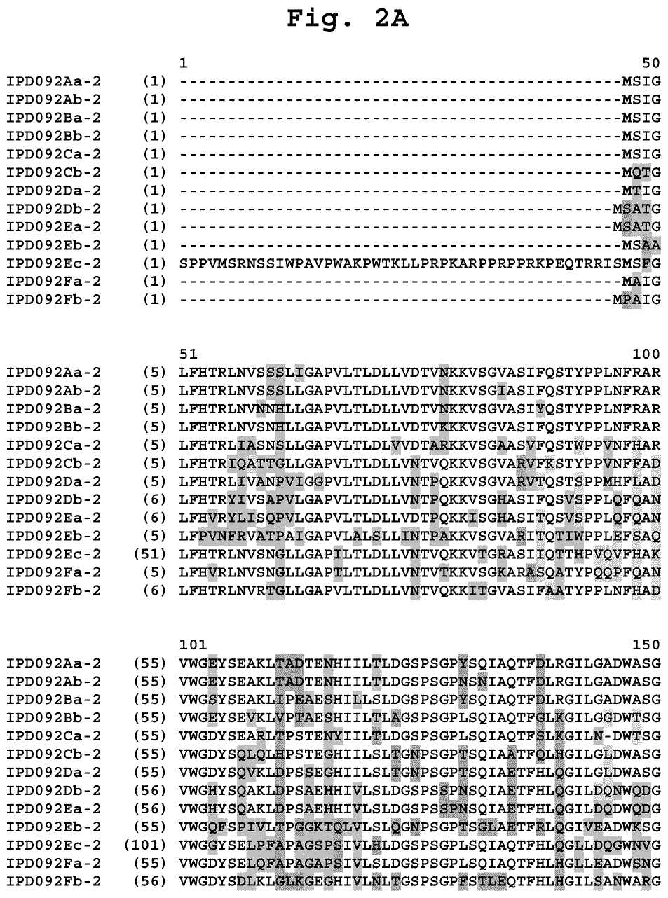

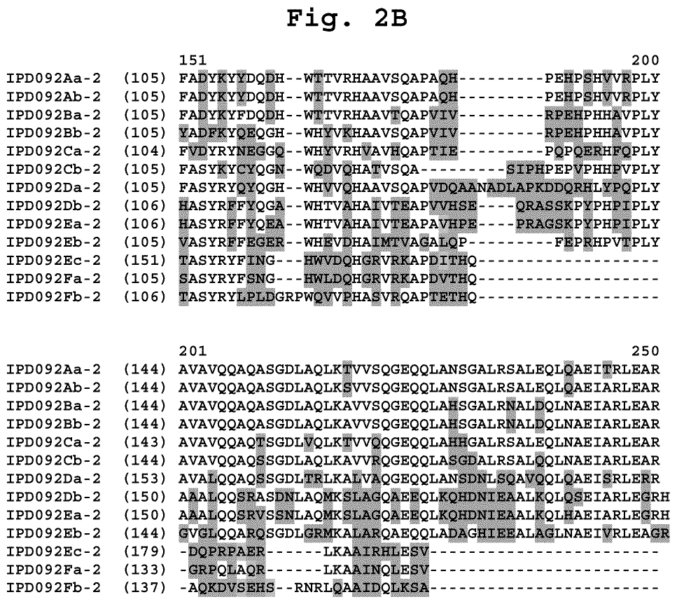

“IPD092-2 polypeptide”, and “IPD092-2 protein” as used herein interchangeably refers to a polypeptide having insecticidal activity, in combination with a IPD092-1 polypeptide, against one or more insect pests of the Lepidoptera and/or Coleoptera orders including but not limited to Western corn rootworm (WCRW), and is sufficiently homologous to the IPD092-2 polypeptide of SEQ ID NO: 2. A variety of IPD092-2 polypeptide homologs are contemplated. Sources of IPD092-2 polypeptide homologs or related proteins include bacterial species selected from but not limited to Pseudomonas species, Chromobacterium species, Burkholderia species, and Woodsholea species. Alignment of the amino acid sequences of IPD092-2 polypeptide homologs (for example—FIG. 2A-2B), allows for the identification of residues that are highly conserved amongst the natural homologs of this family.

In some embodiments, the sequence homology is against the full-length sequence of an

IPD092-2 polypeptide. In some embodiments, the IPD092-2 polypeptide has at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 21, SEQ ID NO: 23 or SEQ ID NO: 25.

In some embodiments an IPD092-2 polypeptide comprises an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 21, SEQ ID NO: 23 or SEQ ID NO: 25.

In some embodiments an IPD092-2 polypeptide comprises an amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 21, SEQ ID NO: 23 or SEQ ID NO: 25 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more amino acid substitutions compared to the native amino acid at the corresponding position of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 21, SEQ ID NO: 23 or SEQ ID NO: 25.

In some embodiments, the IPD092-2 polypeptide comprises the amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 21, SEQ ID NO: 23 or SEQ ID NO: 25.

### IPD095-1 and IPD095-2 Proteins and Variants and Fragments Thereof

IPD095-1 and IPD095-2 polypeptides are encompassed by the disclosure. “IPD095-1 polypeptide”, and “IPD095-1 protein” as used herein interchangeably refers to a polypeptide having insecticidal activity, in combination with a IPD095-2 polypeptide, against one or more insect pests of the Lepidoptera and/or Coleoptera orders including but not limited to Western corn rootworm (WCRVV), and is sufficiently homologous to the IPD095-1 polypeptide of SEQ ID NO: 27. A variety of IPD095-1 polypeptide homologs are contemplated. Sources of IPD095-1 polypeptide homologs or related proteins include bacterial species selected from but not limited to Serratia species, Leminorella species, Dickeya species, Enterobacter species, Erwinia species, Yersinia species, and Rahnella species. Alignment of the amino acid sequences of IPD095-1 polypeptide homologs allows for the identification of residues that are highly conserved amongst the natural homologs of this family. IPD095-1 homologs can be aligned in a similar manner as shown in FIGS. 1 and 2 for IPD092-1 and IPD092-2 homologs to identify conserved amino acid positions, positions tolerant to change, motifs, and domains.

In some embodiments, the IPD095-1 polypeptide has at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to SEQ ID NO: 27, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 or SEQ ID NO: 58.

In some embodiments, the sequence homology is against the full-length sequence of an IPD095-1 polypeptide. In some embodiments an IPD095-1 polypeptide comprises an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 27, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 or SEQ ID NO: 58.

In some embodiments an IPD095-1 polypeptide comprises an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 27, SEQ ID NO: 29, SEQ ID NO: 30, SEQ ID NO: 34, SEQ ID NO: 35, SEQ ID NO: 38, SEQ ID NO: 41, SEQ ID NO: 42, SEQ ID NO: 43, SEQ ID NO: 48, SEQ ID NO: 49, SEQ ID NO: 50, SEQ ID NO: 51, SEQ ID NO: 54, SEQ ID NO: 56 or SEQ ID NO: 57.

In some embodiments an IPD095-1 polypeptide comprises an amino acid sequence of

SEQ ID NO: 27, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 or SEQ ID NO: 58 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85 or more amino acid substitutions compared to the native amino acid at the corresponding position of SEQ ID NO: 27, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 or SEQ ID NO: 58.

In some embodiments, the IPD095-1 polypeptide comprises the amino acid sequence of SEQ ID NO: 27, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 or SEQ ID NO: 58.

In some embodiments, the IPD095-1 polypeptide comprises the amino acid sequence of SEQ ID NO: 27, SEQ ID NO: 29, SEQ ID NO: 30, SEQ ID NO: 34, SEQ ID NO: 35, SEQ ID NO: 38, SEQ ID NO: 41, SEQ ID NO: 42, SEQ ID NO: 43, SEQ ID NO: 48, SEQ ID NO: 49, SEQ ID NO: 50, SEQ ID NO: 51, SEQ ID NO: 54, SEQ ID NO: 56 or SEQ ID NO: 57.

“IPD095-2 polypeptide”, and “IPD095-2 protein” as used herein interchangeably refers to a polypeptide having insecticidal activity, in combination with a IPD095-1 polypeptide, against one or more insect pests of the Lepidoptera and/or Coleoptera orders including but not limited to Western corn rootworm (WCRW), and is sufficiently homologous to the IPD095-2 polypeptide of SEQ ID NO: 28. A variety of IPD095-2 polypeptide homologs are contemplated. Sources of IPD095-2 polypeptide homologs or related proteins include bacterial species selected from but not limited to Serratia species, Leminorella species, Dickeya species, Enterobacter species, Erwinia species, Yersinia species, and Rahnella species. Alignment of the amino acid sequences of IPD095-2 polypeptide homologs allows for the identification of residues that are highly conserved amongst the natural homologs of this family. IPD095-2 homologs can be aligned in a similar manner as shown in FIGS. 1 and 2 for IPD092-1 and IPD092-2 homologs to identify conserved amino acid positions, positions tolerant to change, motifs, and domains.

In some embodiments, the sequence homology is against the full-length sequence of an IPD095-2 polypeptide. In some embodiments, the IPD095-2 polypeptide has at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to SEQ ID NO: 28, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119 or SEQ ID NO: 120.

In some embodiments an IPD095-2 polypeptide comprises an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 28, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID

NO: 114, SEQ ID NO: 115, SEQ ID NO: 116, SEQ ID NO: 117, SEQ ID NO: 118, SEQ ID NO: 119 or SEQ ID NO: 120.

In some embodiments an IPD095-2 polypeptide comprises an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 28, SEQ ID NO: 59, SEQ ID NO: 60, SEQ ID NO: 61, SEQ ID NO: 63, SEQ ID NO: 66, SEQ ID NO: 67, SEQ ID NO: 68, SEQ ID NO: 75, SEQ ID NO: 77, SEQ ID NO: 78, SEQ ID NO: 79, SEQ ID NO: 82, SEQ ID NO: 83, SEQ ID NO: 84, SEQ ID NO: 85, SEQ ID NO: 86, SEQ ID NO: 94, SEQ ID NO: 95, SEQ ID NO: 96, SEQ ID NO: 97, SEQ ID NO: 99, SEQ ID NO: 100, SEQ ID NO: 103, SEQ ID NO: 107, SEQ ID NO: 119 or SEQ ID NO: 120.

In some embodiments an IPD095-2 polypeptide comprises an amino acid sequence of SEQ ID NO: 28, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119 or SEQ ID NO: 120 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85 or more amino acid substitutions compared to the native amino acid at the corresponding position of SEQ ID NO: 28, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119 or SEQ ID NO: 120.

In some embodiments, the IPD095-2 polypeptide comprises the amino acid sequence of SEQ ID NO: 28, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119 or SEQ ID NO: 120.

In some embodiments, the IPD095-2 polypeptide comprises the amino acid sequence of SEQ ID NO: 28, SEQ ID NO: 59, SEQ ID NO: 60, SEQ ID NO: 61, SEQ ID NO: 63, SEQ ID NO: 66, SEQ ID NO: 67, SEQ ID NO: 68, SEQ ID NO: 75, SEQ ID NO: 77, SEQ ID NO: 78, SEQ ID NO: 79, SEQ ID NO: 82, SEQ ID NO: 83, SEQ ID NO: 84, SEQ ID NO: 85, SEQ ID NO: 86, SEQ ID NO: 94, SEQ ID NO: 95, SEQ ID NO: 96, SEQ ID NO: 97, SEQ ID NO: 99, SEQ ID NO: 100, SEQ ID NO: 103, SEQ ID NO: 107, SEQ ID NO: 119 or SEQ ID NO: 120.

### IPD097 Proteins and Variants and Fragments Thereof

IPD097 polypeptides are encompassed by the disclosure. “IPD097 polypeptide”, and “IPD097 protein” as used herein interchangeably refers to a polypeptide having insecticidal activity, against one or more insect pests of the Lepidoptera and/or Coleoptera orders including but not limited to Western corn rootworm (WCRW), and is sufficiently homologous to the IPD097 polypeptide of SEQ ID NO: 121. A variety of IPD097 polypeptide homologs are contemplated. Sources of IPD097 polypeptide homologs or related proteins include bacterial species selected from but not limited to Haemophilus species, Aeromonas species, and Clostridiales species. Alignment of the amino acid sequences of IPD097 polypeptide homologs allows for the identification of residues that are highly conserved amongst the natural homologs of this family. IPD097 homologs can be aligned in a similar manner as shown in FIGS. 1 and 2 for IPD092-1 and IPD092-2 homologs to identify conserved amino acid positions, positions tolerant to change, motifs, and domains.

In some embodiments, the sequence homology is against the full-length sequence of an IPD097 polypeptide. In some embodiments, the IPD097 polypeptide has at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134 or SEQ ID NO: 135.

In some embodiments an IPD097 polypeptide comprises an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134 or SEQ ID NO: 135.

In some embodiments an IPD097 polypeptide comprises an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 121, SEQ ID NO: 123, SEQ ID NO: 127, SEQ ID NO: 128, SEQ ID NO: 129, SEQ ID NO: 131 or SEQ ID NO: 132.

In some embodiments an IPD097 polypeptide comprises an amino acid sequence of SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134 or SEQ ID NO: 135 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more amino acid substitutions compared to the native amino acid at the corresponding position of SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134 or SEQ ID NO: 135.

In some embodiments, the IPD097 polypeptide comprises the amino acid sequence of SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134 or SEQ ID NO: 135.

In some embodiments, the IPD097 polypeptide comprises the amino acid sequence of SEQ ID NO: 121, SEQ ID NO: 123, SEQ ID NO: 127, SEQ ID NO: 128, SEQ ID NO: 129, SEQ ID NO: 131 or SEQ ID NO: 132.

### IPD099-1, IPD099-2 and IPD099-3 Proteins and Variants and Fragments Thereof

IPD099-1, IPD099-2 and IPD099-3 polypeptides are encompassed by the disclosure. “IPD099-1 polypeptide”, and “IPD099-1 protein” as used herein interchangeably refers to a polypeptide having insecticidal activity, in combination with a IPD099-2 polypeptide and IPD099-3, against one or more insect pests of the Lepidoptera and/or Coleoptera orders including but not limited to Western corn rootworm (WCRW), and is sufficiently homologous to the IPD099-1 polypeptide of SEQ ID NO: 136. A variety of IPD099-1 polypeptide homologs are contemplated. Sources of IPD099-1 polypeptide homologs or related proteins include bacterial species selected from but not limited to Aeromonas species, Haemophilus species, Burkholderia species, Chromobacterium species, Erwinia species, Serratia species, Salinivibrio species, Aquimarina species, Janthinobacterium species, Tolypothrix species, Photobacterium species, Janthinobacterium species, Rhizobium species, Moritella species, Providencia species, Yersinia species and Vibrio species. Alignment of the amino acid sequences of IPD099-1 polypeptide homologs allows for the identification of residues that are highly conserved amongst the natural homologs of this family. IPD099-1 homologs can be aligned in a similar manner as shown in FIGS. 1 and 2 for IPD092-1 and IPD092-2 homologs to identify conserved amino acid positions, positions tolerant to change, motifs, and domains.

In some embodiments, the sequence homology is against the full-length sequence of an IPD099-1 polypeptide. In some embodiments, the IPD099-1 polypeptide has at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to SEQ ID NO: 136, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214 or SEQ ID NO: 215.

In some embodiments an IPD099-1 polypeptide comprises an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 136, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214 or SEQ ID NO: 215.

In some embodiments an IPD099-1 polypeptide comprises an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 136, SEQ ID NO: 139, SEQ ID NO: 140, SEQ ID NO: 142, SEQ ID NO: 144, SEQ ID NO: 145, SEQ ID NO: 148, SEQ ID NO: 149, SEQ ID NO: 150, SEQ ID NO: 155, SEQ ID NO: 157, SEQ ID NO: 158, SEQ ID NO: 177, SEQ ID NO: 179, SEQ ID NO: 180, SEQ ID NO: 183, SEQ ID NO: 185, SEQ ID NO: 186, SEQ ID NO: 187, SEQ ID NO: 188, SEQ ID NO: 189, SEQ ID NO: 192, SEQ ID NO: 195, SEQ ID NO: 196, SEQ ID NO: 197, SEQ ID NO: 199, SEQ ID NO: 205, SEQ ID NO: 208, SEQ ID NO: 209, SEQ ID NO: 210 or SEQ ID NO: 215.

In some embodiments an IPD099-1 polypeptide comprises an amino acid sequence of SEQ ID NO: 136, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214 or SEQ ID NO: 215 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more amino acid substitutions compared to the native amino acid at the corresponding position of SEQ ID NO: 136, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214 or SEQ ID NO: 215.

In some embodiments, the IPD099-1 polypeptide comprises the amino acid sequence of SEQ ID NO: 136, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214 or SEQ ID NO: 215.

In some embodiments, the IPD099-1 polypeptide comprises the amino acid sequence of SEQ ID NO: 136, SEQ ID NO: 139, SEQ ID NO: 140, SEQ ID NO: 142, SEQ ID NO: 144, SEQ ID NO: 145, SEQ ID NO: 148, SEQ ID NO: 149, SEQ ID NO: 150, SEQ ID NO: 155, SEQ ID NO: 157, SEQ ID NO: 158, SEQ ID NO: 177, SEQ ID NO: 179, SEQ ID NO: 180, SEQ ID NO: 183, SEQ ID NO: 185, SEQ ID NO: 186, SEQ ID NO: 187, SEQ ID NO: 188, SEQ ID NO: 189, SEQ ID NO: 192, SEQ ID NO: 195, SEQ ID NO: 196, SEQ ID NO: 197, SEQ ID NO: 199, SEQ ID NO: 205, SEQ ID NO: 208, SEQ ID NO: 209, SEQ ID NO: 210 or SEQ ID NO: 215.

“IPD099-2 polypeptide”, and “IPD099-2 protein” as used herein interchangeably refers to a polypeptide having insecticidal activity, against one or more insect pests of the Lepidoptera and/or Coleoptera orders including but not limited to Western corn rootworm (WCRW), and is sufficiently homologous to the IPD099-2 polypeptide of SEQ ID NO: 137. A variety of IPD099-2 polypeptide homologs are contemplated. Sources of IPD099-2 polypeptide homologs or related proteins include bacterial species selected from but not limited to Aeromonas species, Haemophilus species, Burkholderia species, Chromobacterium species, Erwinia species, Serratia species, Salinivibrio species, Aquimarina species, Janthinobacterium species, Tolypothrix species, Photobacterium species, Janthinobacterium species, Rhizobium species, Moritella species, Providencia species, Yersinia species and Vibrio species. Alignment of the amino acid sequences of IPD099-2 polypeptide homologs allows for the identification of residues that are highly conserved amongst the natural homologs of this family. IPD099-2 homologs can be aligned in a similar manner as shown in FIGS. 1 and 2 for IPD092-1 and IPD092-2 homologs to identify conserved amino acid positions, positions tolerant to change, motifs, and domains.

In some embodiments, the sequence homology is against the full-length sequence of an IPD099-2 polypeptide. In some embodiments, the IPD099-2 polypeptide has at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to SEQ ID NO: 137, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270 or SEQ ID NO: 271.

In some embodiments an IPD099-2 polypeptide comprises an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 137, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270 or SEQ ID NO: 271.

In some embodiments an IPD099-2 polypeptide comprises an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 137, SEQ ID NO: 216, SEQ ID NO: 221, SEQ ID NO: 222, SEQ ID NO: 225, SEQ ID NO: 227, SEQ ID NO: 230, SEQ ID NO: 232, SEQ ID NO: 244, SEQ ID NO: 246, SEQ ID NO: 249, SEQ ID NO: 251, SEQ ID NO: 254, SEQ ID NO: 255, SEQ ID NO: 262, SEQ ID NO: 265, SEQ ID NO: 268 or SEQ ID NO: 269.

In some embodiments an IPD099-2 polypeptide comprises an amino acid sequence of SEQ ID NO: 137, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270 or SEQ ID NO: 271 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more amino acid substitutions compared to the native amino acid at the corresponding position of SEQ ID NO: 137, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270 or SEQ ID NO: 271.

In some embodiments, the IPD099-2 polypeptide comprises the amino acid sequence of SEQ ID NO: 137, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270 or SEQ ID NO: 271.

In some embodiments, the IPD099-2 polypeptide comprises the amino acid sequence of SEQ ID NO: 137, SEQ ID NO: 216, SEQ ID NO: 221, SEQ ID NO: 222, SEQ ID NO: 225, SEQ ID NO: 227, SEQ ID NO: 230, SEQ ID NO: 232, SEQ ID NO: 244, SEQ ID NO: 246, SEQ ID NO: 249, SEQ ID NO: 251, SEQ ID NO: 254, SEQ ID NO: 255, SEQ ID NO: 262, SEQ ID NO: 265, SEQ ID NO: 268 or SEQ ID NO: 269.

“IPD099-3 polypeptide”, and “IPD099-3 protein” as used herein interchangeably refers to a polypeptide having insecticidal activity, in combination with an IPD099-1 polypeptide and an IPD099-2 polypeptide, against one or more insect pests of the Lepidoptera and/or Coleoptera orders including but not limited to Western corn rootworm (WCRW), and is sufficiently homologous to the IPD099-3 polypeptide of SEQ ID NO: 138. A variety of IPD099-3 polypeptide homologs are contemplated. Sources of IPD099-3 polypeptide homologs or related proteins include bacterial species selected from but not limited to Aeromonas species, Haemophilus species, Burkholderia species, Chromobacterium species, Erwinia species, Serratia species, Salinivibrio species, Aquimarina species, Janthinobacterium species, Tolypothrix species, Photobacterium species, Janthinobacterium species, Rhizobium species, Moritella species, Providencia species, Yersinia species and Vibrio species. Alignment of the amino acid sequences of IPD099-3 polypeptide homologs allows for the identification of residues that are highly conserved amongst the natural homologs of this family IPD099-3 homologs can be aligned in a similar manner as shown in FIGS. 1 and 2 for IPD092-1 and IPD092-2 homologs to identify conserved amino acid positions, positions tolerant to change, motifs, and domains.

In some embodiments, the sequence homology is against the full-length sequence of an IPD099-3 polypeptide. In some embodiments, the IPD099-3 polypeptide has at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to SEQ ID NO: 138, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330 or SEQ ID NO: 331.

In some embodiments an IPD099-3 polypeptide comprises an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 138, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330 or SEQ ID NO: 331.

In some embodiments an IPD099-3 polypeptide comprises an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 138, SEQ ID NO: 272, SEQ ID NO: 273, SEQ ID NO: 275, SEQ ID NO: 285, SEQ ID NO: 286, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 298, SEQ ID NO: 299, SEQ ID NO: 300, SEQ ID NO: 301, SEQ ID NO: 302, SEQ ID NO: 304, SEQ ID NO: 306, SEQ ID NO: 307, SEQ ID NO: 308, SEQ ID NO: 312, SEQ ID NO: 313, SEQ ID NO: 320, SEQ ID NO: 323, SEQ ID NO: 324, SEQ ID NO: 326 or SEQ ID NO: 331.

In some embodiments an IPD099-3 polypeptide comprises an amino acid sequence of SEQ ID NO: 138, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330 or SEQ ID NO: 331 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more amino acid substitutions compared to the native amino acid at the corresponding position of SEQ ID NO: 138, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330 or SEQ ID NO: 331.

In some embodiments, the IPD099-3 polypeptide comprises the amino acid sequence of SEQ ID NO: 138, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330 or SEQ ID NO: 331.

In some embodiments, the IPD099-3 polypeptide comprises the amino acid sequence of SEQ ID NO: 138, SEQ ID NO: 272, SEQ ID NO: 273, SEQ ID NO: 275, SEQ ID NO: 285, SEQ ID NO: 286, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 298, SEQ ID NO: 299, SEQ ID NO: 300, SEQ ID NO: 301, SEQ ID NO: 302, SEQ ID NO: 304, SEQ ID NO: 306, SEQ ID NO: 307, SEQ ID NO: 308, SEQ ID NO: 312, SEQ ID NO: 313, SEQ ID NO: 320, SEQ ID NO: 323, SEQ ID NO: 324, SEQ ID NO: 326, SEQ ID NO: 331.

### IPD100-1 and IPD100-2 Proteins and Variants and Fragments Thereof

IPD100-1 and IPD100-2 polypeptides are encompassed by the disclosure. “IPD100-1 polypeptide”, and “IPD100-1 protein” as used herein interchangeably refers to a polypeptide having insecticidal activity, in combination with a IPD100-2 polypeptide, against one or more insect pests of the Lepidoptera and/or Coleoptera orders including but not limited to Western corn rootworm (WCRVV), and is sufficiently homologous to the IPD100-1 polypeptide of SEQ ID NO: 332. A variety of IPD100-1 polypeptide homologs are contemplated. Sources of IPD100-1 polypeptide homologs or related proteins include bacterial species selected from but not limited to Pseudomonas species, Candidatus species, Burkholderia species, Duganella species, Salmonella species, Tenacibaculum species, Dickeya species, Pedobacter species, and Mycobacterium species. Alignment of the amino acid sequences of IPD100-1 polypeptide homologs allows for the identification of residues that are highly conserved amongst the natural homologs of this family. IPD100-1 homologs can be aligned in a similar manner as shown in FIGS. 1 and 2 for IPD092-1 and IPD092-2 homologs to identify conserved amino acid positions, positions tolerant to change, motifs, and domains. In some embodiments, the sequence homology is against the full-length sequence of an IPD100-1 polypeptide. In some embodiments, the IPD100-1 polypeptide has at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to SEQ ID NO: 332, SEQ ID NO: 334, SEQ ID NO: 335 or SEQ ID NO: 336.

In some embodiments an IPD100-1 polypeptide comprises an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 332, SEQ ID NO: 334, SEQ ID NO: 335 or SEQ ID NO: 336.

In some embodiments, an IPD100-1 polypeptide comprises an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 332.

In some embodiments an IPD100-1 polypeptide comprises an amino acid sequence of SEQ ID NO: 332, SEQ ID NO: 334, SEQ ID NO: 335 or SEQ ID NO: 336 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75 or more amino acid substitutions compared to the native amino acid at the corresponding position of SEQ ID NO: 332, SEQ ID NO: 334, SEQ ID NO: 335 or SEQ ID NO: 336.

In some embodiments, the IPD100-1 polypeptide comprises the amino acid sequence of SEQ ID NO: 332, SEQ ID NO: 334, SEQ ID NO: 335 or SEQ ID NO: 336.

In some embodiments, the IPD100-1 polypeptide comprises the amino acid sequence of SEQ ID NO: 332.

“IPD100-2 polypeptide”, and “IPD100-2 protein” as used herein interchangeably refers to a polypeptide having insecticidal activity, in combination with a IPD100-1 polypeptide, against one or more insect pests of the Lepidoptera and/or Coleoptera orders including but not limited to Western corn rootworm (WCRW), and is sufficiently homologous to the IPD100-2 polypeptide of SEQ ID NO: 333. A variety of IPD100-2 polypeptide homologs are contemplated. Sources of IPD100-2 polypeptide homologs or related proteins include bacterial species selected from but not limited to Pseudomonas species, Candidatus species, Burkholderia species, Duganella species, Salmonella species, Tenacibaculum species, Dickeya species, Pedobacter species, and Mycobacterium species. Alignment of the amino acid sequences of IPD100-2 polypeptide homologs allows for the identification of residues that are highly conserved amongst the natural homologs of this family. IPD100-2 homologs can be aligned in a similar manner as shown in FIGS. 1 and 2 for IPD092-1 and IPD092-2 homologs to identify conserved amino acid positions, positions tolerant to change, motifs, and domains.

In some embodiments, the sequence homology is against the full-length sequence of an IPD100-2 polypeptide. In some embodiments, the IPD100-2 polypeptide has at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to SEQ ID NO: 333, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348 or SEQ ID NO: 349.

In some embodiments an IPD100-2 polypeptide comprises an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 333, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348 or SEQ ID NO: 349.

In some embodiments an IPD100-2 polypeptide comprises an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 333, SEQ ID NO: 337, SEQ ID NO: 338, SEQ ID NO: 341, SEQ ID NO: 342, SEQ ID NO: 343, SEQ ID NO: 344 or SEQ ID NO: 347.

In some embodiments an IPD100-2 polypeptide comprises an amino acid sequence of SEQ ID NO: 333, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348 or SEQ ID NO: 349 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more amino acid substitutions compared to the native amino acid at the corresponding position of SEQ ID NO: 333, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348 or SEQ ID NO: 349.

In some embodiments, the IPD100-2 polypeptide comprises the amino acid sequence of SEQ ID NO: 333, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348 or SEQ ID NO: 349.

In some embodiments, the IPD100-2 polypeptide comprises the amino acid sequence of SEQ ID NO: 333, SEQ ID NO: 337, SEQ ID NO: 338, SEQ ID NO: 341, SEQ ID NO: 342, SEQ ID NO: 343, SEQ ID NO: 344 or SEQ ID NO: 347.

### IPD105 Proteins and Variants and Fragments Thereof

IPD105 polypeptides are encompassed by the disclosure. “IPD105 polypeptide”, and “IPD105 protein” as used herein interchangeably refers to a polypeptide having insecticidal activity against one or more insect pests of the Lepidoptera and/or Coleoptera orders including but not limited to Western corn rootworm (WCRW), and is sufficiently homologous to the IPD105 polypeptide of SEQ ID NO: 350. A variety of IPD105 polypeptide homologs are contemplated. Sources of IPD105 polypeptide homologs or related proteins include bacterial species selected from but not limited to Chromobacterium species and Pseudogulbenkiania species. Alignment of the amino acid sequences of IPD105 polypeptide homologs (for example—FIG. 3A-3B), allows for the identification of residues that are highly conserved amongst the natural homologs of this family. IPD105 homologs can be aligned in a similar manner as shown in FIGS. 1 and 2 for IPD092-1 and IPD092-2 homologs to identify conserved amino acid positions, positions tolerant to change, motifs, and domains.

In some embodiments, the sequence homology is against the full-length sequence of an

IPD105 polypeptide. In some embodiments, the IPD105 polypeptide has at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364 or SEQ ID NO: 365.

In some embodiments an IPD105 polypeptide comprises an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364 or SEQ ID NO: 365.

In some embodiments an IPD105 polypeptide comprises an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 350, SEQ ID NO: 353, SEQ ID NO: 355, SEQ ID NO: 357 or SEQ ID NO: 362.

In some embodiments an IPD105 polypeptide comprises an amino acid sequence of SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364 or SEQ ID NO: 365 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more amino acid substitutions compared to the native amino acid at the corresponding position of SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364 or SEQ ID NO: 365.

In some embodiments, the IPD105 polypeptide comprises the amino acid sequence of SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364 or SEQ ID NO: 365.

In some embodiments, the IPD105 polypeptide comprises the amino acid sequence of SEQ ID NO: 350, SEQ ID NO: 353, SEQ ID NO: 355, SEQ ID NO: 357 or SEQ ID NO: 362.

### IPD106-1 and IPD106-2 Proteins and Variants and Fragments Thereof

IPD106-1 and IPD106-2 polypeptides are encompassed by the disclosure. “IPD106-1 polypeptide”, and “IPD106-1 protein” as used herein interchangeably refers to a polypeptide having insecticidal activity, in combination with a IPD106-2 polypeptide, against one or more insect pests of the Lepidoptera and/or Coleoptera orders including but not limited to Western corn rootworm (WCRW), and is sufficiently homologous to the IPD106-1 polypeptide of SEQ ID NO: 366. A variety of IPD106-1 polypeptide homologs are contemplated. Sources of IPD106-1 polypeptide homologs or related proteins include bacterial species selected from but not limited to Arsenicibacter species and Chitinophaga species. Alignment of the amino acid sequences of IPD106-1 polypeptide homologs allows for the identification of residues that are highly conserved amongst the natural homologs of this family. IPD106-1 homologs can be aligned in a similar manner as shown in FIGS. 1 and 2 for IPD092-1 and IPD092-2 homologs to identify conserved amino acid positions, positions tolerant to change, motifs, and domains.

In some embodiments, the sequence homology is against the full-length sequence of an IPD106-1 polypeptide. In some embodiments, the IPD106-1 polypeptide has at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to SEQ ID NO: 366, SEQ ID NO: 368, SEQ ID NO: 369, SEQ ID NO: 370 or SEQ ID NO: 371.

In some embodiments an IPD106-1 polypeptide comprises an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 366, SEQ ID NO: 368, SEQ ID NO: 369, SEQ ID NO: 370 or SEQ ID NO: 371.

In some embodiments an IPD106-1 polypeptide comprises an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 366, SEQ ID NO: 368 or SEQ ID NO: 369.

In some embodiments an IPD106-1 polypeptide comprises an amino acid sequence of SEQ ID NO: 366, SEQ ID NO: 368, SEQ ID NO: 369, SEQ ID NO: 370 or SEQ ID NO: 371 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more amino acid substitutions compared to the native amino acid at the corresponding position of SEQ ID NO: 366, SEQ ID NO: 368, SEQ ID NO: 369, SEQ ID NO: 370 or SEQ ID NO: 371.

In some embodiments, the IPD106-1 polypeptide comprises the amino acid sequence of SEQ ID NO: 366, SEQ ID NO: 368, SEQ ID NO: 369, SEQ ID NO: 370 or SEQ ID NO: 371.

In some embodiments, the IPD106-1 polypeptide comprises the amino acid sequence of SEQ ID NO: 366, SEQ ID NO: 368 or SEQ ID NO: 369.

“IPD106-2 polypeptide”, and “IPD106-2 protein” as used herein interchangeably refers to a polypeptide having insecticidal activity, in combination with a IPD106-1 polypeptide, against one or more insect pests of the Lepidoptera and/or Coleoptera orders including but not limited to Western corn rootworm (WCRW), and is sufficiently homologous to the IPD106-2 polypeptide of SEQ ID NO: 367. A variety of IPD106-2 polypeptide homologs are contemplated. Sources of IPD106-2 polypeptide homologs or related proteins include bacterial species selected from but not limited to Arsenicibacter species and Chitinophaga species. Alignment of the amino acid sequences of IPD106-2 polypeptide homologs allows for the identification of residues that are highly conserved amongst the natural homologs of this family. IPD106-2 homologs can be in a similar manner as shown in FIGS. 1 and 2 for IPD092-1 and IPD092-2 homologs to identify conserved amino acid positions, positions tolerant to change, motifs, and domains.

In some embodiments, the sequence homology is against the full-length sequence of an IPD106-2 polypeptide. In some embodiments, the IPD106-2 polypeptide has at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to SEQ ID NO: 367, SEQ ID NO: 372, SEQ ID NO: 373, SEQ ID NO: 374, SEQ ID NO: 375 or SEQ ID NO: 376.

In some embodiments an IPD106-2 polypeptide comprises an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 367, SEQ ID NO: 372, SEQ ID NO: 373, SEQ ID NO: 374, SEQ ID NO: 375 or SEQ ID NO: 376.

In some embodiments an IPD106-2 polypeptide comprises an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 367, SEQ ID NO: 372, SEQ ID NO: 373 or SEQ ID NO: 376.

In some embodiments an IPD106-2 polypeptide comprises an amino acid sequence of SEQ ID NO: 367, SEQ ID NO: 372, SEQ ID NO: 373, SEQ ID NO: 374, SEQ ID NO: 375 or SEQ ID NO: 376 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more amino acid substitutions compared to the native amino acid at the corresponding position of SEQ ID NO: 367, SEQ ID NO: 372, SEQ ID NO: 373, SEQ ID NO: 374, SEQ ID NO: 375 or SEQ ID NO: 376.

In some embodiments, the IPD106-2 polypeptide comprises the amino acid sequence of SEQ ID NO: 367, SEQ ID NO: 372, SEQ ID NO: 373, SEQ ID NO: 374, SEQ ID NO: 375 or SEQ ID NO: 376.

In some embodiments, the IPD106-2 polypeptide comprises the amino acid sequence of SEQ ID NO: 367, SEQ ID NO: 372, SEQ ID NO: 373 or SEQ ID NO: 376.

### IPD107 Proteins and Variants and Fragments Thereof

IPD107 polypeptides are encompassed by the disclosure. “IPD107 polypeptide”, and “IPD107 protein” as used herein interchangeably refers to a polypeptide having insecticidal activity against one or more insect pests of the Lepidoptera and/or Coleoptera orders including but not limited to Western corn rootworm (WCRW), and is sufficiently homologous to the IPD107 polypeptide of SEQ ID NO: 377. A variety of IPD107 polypeptide homologs are contemplated. Sources of IPD107 polypeptide homologs or related proteins include bacterial species selected from but not limited to Pseudomonas species, Chromobacterium species, and Bradyrhizobium species. Alignment of the amino acid sequences of IPD107 polypeptide homologs allows for the identification of residues that are highly conserved amongst the natural homologs of this family. IPD107 homologs can be aligned in a similar manner as shown in FIGS. 1 and 2 for IPD092-1 and IPD092-2 homologs, to identify conserved amino acid positions, positions tolerant to change, motifs, and domains.

In some embodiments, the sequence homology is against the full-length sequence of an IPD107 polypeptide. In some embodiments, the IPD107 polypeptide has at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451 or SEQ ID NO: 452.

In some embodiments an IPD107 polypeptide comprises an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451 or SEQ ID NO: 452.

In some embodiments an IPD107 polypeptide comprises an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 377, SEQ ID NO: 378, SEQ ID NO: 381, SEQ ID NO: 382, SEQ ID NO: 384, SEQ ID NO: 386, SEQ ID NO: 387, SEQ ID NO: 388, SEQ ID NO: 389, SEQ ID NO: 390, SEQ ID NO: 391, SEQ ID NO: 393, SEQ ID NO: 396, SEQ ID NO: 397, SEQ ID NO: 398, SEQ ID NO: 400, SEQ ID NO: 401, SEQ ID NO: 402, SEQ ID NO: 403, SEQ ID NO: 404, SEQ ID NO: 405, SEQ ID NO: 407, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 417, SEQ ID NO: 418, SEQ ID NO: 419, SEQ ID NO: 420, SEQ ID NO: 421, SEQ ID NO: 422, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 438, SEQ ID NO: 439, SEQ ID NO: 440, SEQ ID NO: 442, SEQ ID NO: 443, SEQ ID NO: 445, SEQ ID NO: 446, SEQ ID NO: 451 or SEQ ID NO: 452.

In some embodiments an IPD107 polypeptide comprises an amino acid sequence of SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451 or SEQ ID NO: 452 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more amino acid substitutions compared to the native amino acid at the corresponding position of SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451 or SEQ ID NO: 452.

In some embodiments, the IPD107 polypeptide comprises the amino acid sequence of SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451 or SEQ ID NO: 452.

In some embodiments, the IPD107 polypeptide comprises the amino acid sequence of SEQ ID NO: 377, SEQ ID NO: 378, SEQ ID NO: 381, SEQ ID NO: 382, SEQ ID NO: 384, SEQ ID NO: 386, SEQ ID NO: 387, SEQ ID NO: 388, SEQ ID NO: 389, SEQ ID NO: 390, SEQ ID NO: 391, SEQ ID NO: 393, SEQ ID NO: 396, SEQ ID NO: 397, SEQ ID NO: 398, SEQ ID NO: 400, SEQ ID NO: 401, SEQ ID NO: 402, SEQ ID NO: 403, SEQ ID NO: 404, SEQ ID NO: 405, SEQ ID NO: 407, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 417, SEQ ID NO: 418, SEQ ID NO: 419, SEQ ID NO: 420, SEQ ID NO: 421, SEQ ID NO: 422, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 438, SEQ ID NO: 439, SEQ ID NO: 440, SEQ ID NO: 442, SEQ ID NO: 443, SEQ ID NO: 445, SEQ ID NO: 446, SEQ ID NO: 451 or SEQ ID NO: 452.

### IPD111 Proteins and Variants and Fragments Thereof

IPD111 polypeptides are encompassed by the disclosure. “IPD111 polypeptide”, and

“IPD111 protein” as used herein interchangeably refers to a polypeptide having insecticidal activity against one or more insect pests of the Lepidoptera and/or Coleoptera orders including but not limited to Western corn rootworm (WCRW), and is sufficiently homologous to the IPD111 polypeptide of SEQ ID NO: 453. A variety of IPD111 polypeptide homologs are contemplated. Sources of IPD111 polypeptide homologs or related proteins include bacterial species selected from but not limited to Pseudomonas species, Chromobacterium species, and Burkholderia species. Alignment of the amino acid sequences of IPD111 polypeptide homologs allows for the identification of residues that are highly conserved amongst the natural homologs of this family. IPD111 homologs can be aligned in a similar manner as shown in FIGS. 1 and 2 for IPD092-1 and IPD092-2 homologs, to identify conserved amino acid positions, positions tolerant to change, motifs, and domains.

In some embodiments, the sequence homology is against the full-length sequence of an IPD111 polypeptide. In some embodiments, the IPD111 polypeptide has at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to SEQ ID NO: 453, SEQ ID NO: 454, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524, SEQ ID NO: 525, SEQ ID NO: 526, SEQ ID NO: 527 or SEQ ID NO: 528.

In some embodiments an IPD111 polypeptide comprises an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 453, SEQ ID NO: 454, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524, SEQ ID NO: 525, SEQ ID NO: 526, SEQ ID NO: 527 or SEQ ID NO: 528.

In some embodiments an IPD111 polypeptide comprises an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 453, SEQ ID NO: 454, SEQ ID NO: 455, SEQ ID NO: 456, SEQ ID NO: 462, SEQ ID NO: 463, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 478, SEQ ID NO: 479, SEQ ID NO: 489, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524 or SEQ ID NO: 526.

In some embodiments an IPD111 polypeptide comprises an amino acid sequence of SEQ ID NO: 453, SEQ ID NO: 454, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524, SEQ ID NO: 525, SEQ ID NO: 526, SEQ ID NO: 527 or SEQ ID NO: 528 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more amino acid substitutions compared to the native amino acid at the corresponding position of SEQ ID NO: 453, SEQ ID NO: 454, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524, SEQ ID NO: 525, SEQ ID NO: 526, SEQ ID NO: 527 or SEQ ID NO: 528.

In some embodiments, the IPD111 polypeptide comprises the amino acid sequence of SEQ ID NO: 453, SEQ ID NO: 454, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524, SEQ ID NO: 525, SEQ ID NO: 526, SEQ ID NO: 527 or SEQ ID NO: 528.

In some embodiments, the IPD111 polypeptide comprises the amino acid sequence of SEQ ID NO: 453, SEQ ID NO: 454, SEQ ID NO: 455, SEQ ID NO: 456, SEQ ID NO: 462, SEQ ID NO: 463, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 478, SEQ ID NO: 479, SEQ ID NO: 489, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524 or SEQ ID NO: 526.

### IPD112 Proteins and Variants and Fragments Thereof

IPD112 polypeptides are encompassed by the disclosure. “IPD112 polypeptide”, and “IPD112 protein” as used herein interchangeably refers to a polypeptide having insecticidal activity against one or more insect pests of the Lepidoptera and/or Coleoptera orders including but not limited to Western corn rootworm (WCRW), and is sufficiently homologous to the IPD112 polypeptide of SEQ ID NO: 529. A variety of IPD112 polypeptide homologs are contemplated. Sources of IPD112 polypeptide homologs or related proteins include bacterial species selected from but not limited to Pseudomonas species and Hafnia species. Alignment of the amino acid sequences of IPD112 polypeptide homologs allows for the identification of residues that are highly conserved amongst the natural homologs of this family. IPD112 homologs can be aligned in a similar manner as shown in FIGS. 1 and 2 for IPD092-1 and IPD092-2 homologs, to identify conserved amino acid positions, positions tolerant to change, motifs, and domains.

In some embodiments, the sequence homology is against the full-length sequence of an IPD112 polypeptide. In some embodiments, the IPD112 polypeptide has at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544 or SEQ ID NO: 545.

In some embodiments an IPD112 polypeptide comprises an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544 or SEQ ID NO: 545.

In some embodiments an IPD112 polypeptide comprises an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 529, SEQ ID NO: 530, SEQ ID NO: 531, SEQ ID NO: 532, SEQ ID NO: 534, SEQ ID NO: 537 or SEQ ID NO: 545.

In some embodiments an IPD112 polypeptide comprises an amino acid sequence of SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544 or SEQ ID NO: 545 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more amino acid substitutions compared to the native amino acid at the corresponding position of SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544 or SEQ ID NO: 545.

In some embodiments, the IPD112 polypeptide comprises the amino acid sequence of SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544 or SEQ ID NO: 545.

In some embodiments, the IPD112 polypeptide comprises the amino acid sequence of SEQ ID NO: 529, SEQ ID NO: 530, SEQ ID NO: 531, SEQ ID NO: 532, SEQ ID NO: 534, SEQ ID NO: 537 or SEQ ID NO: 545.

As used herein, the terms “protein,” “peptide molecule,” or “polypeptide” includes any molecule that comprises five or more amino acids. Protein, peptide or polypeptide molecules may undergo modification, including post-translational modifications, such as, but not limited to, disulfide bond formation, glycosylation, phosphorylation or oligomerization. Thus, as used herein, the terms “protein,” “peptide molecule” or “polypeptide” includes any protein that is modified by any biological or non-biological process. The terms “amino acid” and “amino acids” refer to all naturally occurring L-amino acids.

A “recombinant protein” is used herein to refer to a protein that is no longer in its natural environment, for example in vitro or in a recombinant bacterial or plant host cell. A polypeptide of the disclosure that is substantially free of cellular material includes preparations of protein having less than about 30%, 20%, 10% or 5% (by dry weight) of non-pesticidal protein (also referred to herein as a “contaminating protein”).

“Fragments” or “biologically active portions” include polypeptide fragments comprising amino acid sequences sufficiently identical to polypeptides of the disclosure and that exhibit insecticidal activity. “Fragments” or “biologically active portions” of polypeptides of the disclosure includes fragments comprising amino acid sequences sufficiently identical to have insecticidal activity. Such biologically active portions can be prepared by recombinant techniques and evaluated for insecticidal activity. In some embodiments, the polypeptide fragment is an N-terminal and/or a C-terminal truncation of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more amino acids from the N-terminus and/or C-terminus by proteolysis, by insertion of a start codon, by deletion of the codons encoding the deleted amino acids and concomitant insertion of a start codon, and/or insertion of a stop codon.

“Variants” as used herein refers to proteins or polypeptides having an amino acid sequence that is at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or greater identical to the parental amino acid sequence.

Amino acid sequence variants of a polypeptide of the disclosure can be prepared by mutations in the DNA. This may also be accomplished by one of several forms of mutagenesis and/or in directed evolution. In some aspects, the changes encoded in the amino acid sequence will not substantially affect the function of the protein. Such variants will possess the desired pesticidal activity. However, it is understood that the ability of a polypeptide to confer pesticidal activity may be improved using such techniques upon the compositions of this disclosure.

Variants may be made by making random mutations or the variants may be designed. In the case of designed mutants, there is a high probability of generating variants with similar activity to the native toxin when amino acid identity is maintained in critical regions of the toxin which account for biological activity or are involved in the determination of three-dimensional configuration which ultimately is responsible for the biological activity. A high probability of retaining activity will also occur if substitutions are conservative. Amino acids may be placed in the following classes: non-polar, uncharged polar, basic, and acidic. Conservative substitutions whereby an amino acid of one class is replaced with another amino acid of the same type are least likely to materially alter the biological activity of the variant. Table 1 provides a listing of examples of amino acids belonging to each class.

TABLE 1

| Class of Amino Acid | Examples of Amino Acids |
|---|---|
| Nonpolar Side Chains | Ala (A), Val (V), Leu (L), Ile (I), |
| | Pro (P), Met (M), Phe (F), Trp (W) |
| Uncharged Polar Side Chains | Gly (G), Ser (S), Thr (T), Cys (C), |
| | Tyr (Y), Asn (N), Gln (Q) |
| Acidic Side Chains | Asp (D), Glu (E) |
| Basic Side Chains | Lys (K), Arg (R), His (H) |
| Beta-branched Side Chains | Thr, Val, Ile |
| Aromatic Side Chains | Tyr, Phe, Trp, His |

Variant proteins encompassed by the disclosure are biologically active, that is they continue to possess the desired biological activity (i.e. pesticidal activity) of the native protein. In some embodiment, the variant will have at least about 10%, at least about 30%, at least about 50%, at least about 70%, at least about 80% or more of the insecticidal activity of the native protein. In some embodiments, the variants may have improved activity over the native protein.

Variant nucleotide and amino acid sequences of the disclosure also encompass sequences derived from mutagenic and recombinogenic procedures such as DNA shuffling. With such a procedure, one or more different polypeptide coding regions can be used to create a new polypeptide possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between a pesticidal gene and other known pesticidal genes to obtain a new gene coding for a protein with an improved property of interest, such as an increased insecticidal activity. Strategies for such DNA shuffling can be found in Stemmer, (1994) Proc. Natl. Acad. Sci. USA 91:10747-10751; Stemmer, (1994) Nature 370:389-391; Crameri, et al., (1997) Nature Biotech. 15:436-438; Moore, et al., (1997) J. Mol. Biol. 272:336-347; Zhang, et al., (1997) Proc. Natl. Acad. Sci. USA 94:4504-4509; Crameri, et al., (1998) Nature 391:288-291; and U.S. Pat. Nos. 5,605,793 and 5,837,458.

Domain swapping or shuffling is another mechanism for generating altered polypeptides. Domains may be swapped between polypeptides resulting in hybrid or chimeric toxins with improved insecticidal activity or target spectrum. Methods for generating recombinant proteins and testing them for pesticidal activity can be found in Naimov, et al., (2001) Appl. Environ. Microbiol. 67:5328-5330; de Maagd, et al., (1996) Appl. Environ. Microbiol. 62:1537-1543; Ge, et al., (1991) J. Biol. Chem. 266:17954-17958; Schnepf, et al., (1990) J. Biol. Chem. 265:20923-20930; Rang, et al., 91999) Appl. Environ. Microbiol. 65:2918-2925).

A sequence and structure analysis method can be employed, which is composed of four components: phylogenetic tree construction, protein sequence motifs finding, secondary structure prediction, and alignment of protein sequences and secondary structures. Details about each component are illustrated below.

1) Phylogenetic Tree Construction

The phylogenetic analysis can be performed using the software MEGAS. Protein sequences can be subjected to ClustalW version 2 analysis (Larkin M. A et al (2007) Bioinformatics 23(21): 2947-2948) for multiple sequence alignment. The evolutionary history is then inferred by the Maximum Likelihood method based on the JTT matrix-based model. The tree with the highest log likelihood is obtained, exported in Newick format, and further processed to extract the sequence IDs in the same order as they appeared in the tree. A few clades representing sub-families can be manually identified for each insecticidal protein family.

2) Protein Sequence Motifs Finding

Protein sequences are re-ordered according to the phylogenetic tree built previously, and fed to the MOTIF analysis tool MEME (Multiple EM for MOTIF Elicitation) (Bailey T. L., and Elkan C., Proceedings of the Second International Conference on Intelligent Systems for Molecular Biology, pp. 28-36, AAAI Press, Menlo Park, Calif., 1994.) for identification of key sequence motifs. MEME is setup as follows: Minimum number of sites 2, Minimum motif width 5, and Maximum number of motifs 30. Sequence motifs unique to each sub-family were identified by visual observation. The distribution of MOTIFs across the entire gene family could be visualized in HTML webpage. The MOTIFs are numbered relative to the ranking of the E-value for each MOTIF.

3) Secondary Structure Prediction

PSIPRED, top ranked secondary structure prediction method (Jones D T. (1999) J. Mol. Biol. 292: 195-202), can be used for protein secondary structure prediction. The tool provides accurate structure prediction using two feed-forward neural networks based on the PSI-BLAST output. The PSI-BLAST database is created by removing low-complexity, transmembrane, and coiled-coil regions in Uniref100. The PSIPRED results contain the predicted secondary structures (Alpha helix: H, Beta strand: E, and Coil: C) and the corresponding confidence scores for each amino acid in a given protein sequence.

4) Alignment of Protein Sequences and Secondary Structures

A script can be developed to generate gapped secondary structure alignment according to the multiple protein sequence alignment from step 1 for all proteins. All aligned protein sequences and structures are concatenated into a single FASTA file, and then imported into MEGA for visualization and identification of conserved structures.

In some embodiments, the polypeptides of the disclosure have a modified physical property. As used herein, the term “physical property” refers to any parameter suitable for describing the physical-chemical characteristics of a protein. As used herein, “physical property of interest” and “property of interest” are used interchangeably to refer to physical properties of proteins that are being investigated and/or modified. Examples of physical properties include, but are not limited to, net surface charge and charge distribution on the protein surface, net hydrophobicity and hydrophobic residue distribution on the protein surface, surface charge density, surface hydrophobicity density, total count of surface ionizable groups, surface tension, protein size and its distribution in solution, melting temperature, heat capacity, and second virial coefficient. Examples of physical properties also include, polypeptides having increased expression, increased solubility, decreased phytotoxicity, and digestibility of proteolytic fragments in an insect gut. Models for digestion by simulated gastric fluids can be found in Fuchs, R. L. and J. D. Astwood. Food Technology 50: 83-88, 1996; Astwood, J. D., et al Nature Biotechnology 14: 1269-1273, 1996; Fu T J et al J. Agric Food Chem. 50: 7154-7160, 2002).

In some embodiments variants include polypeptides that differ in amino acid sequence due to mutagenesis. Variant proteins encompassed by the disclosure are biologically active, that is they continue to possess the desired biological activity (i.e. pesticidal activity) of the native protein. In some embodiments, the variant will have at least about 10%, at least about 30%, at least about 50%, at least about 70%, at least about 80% or more of the insecticidal activity of the native protein. In some embodiments, the variants may have improved activity over the native protein.

Bacterial genes quite often possess multiple methionine initiation codons in proximity to the start of the open reading frame. Often, translation initiation at one or more of these start codons will lead to generation of a functional protein. These start codons can include ATG codons. However, bacteria such as Bacillus sp. also recognize the codon GTG as a start codon, and proteins that initiate translation at GTG codons contain a methionine at the first amino acid. On rare occasions, translation in bacterial systems can initiate at a TTG codon, though in this event the TTG encodes a methionine. Furthermore, it is not often determined a priori which of these codons are used naturally in the bacterium. Thus, it is understood that use of one of the alternate methionine codons may also lead to generation of pesticidal proteins. These pesticidal proteins are encompassed in the present disclosure and may be used in the methods of the present disclosure. It will be understood that, when expressed in plants, it will be necessary to alter the alternate start codon to ATG for proper translation.

In some embodiments, chimeric polypeptides are provided comprising regions of at least two different IPD092-1 polypeptides, IPD092-2 polypeptides, IPD095-1 polypeptides, IPD095-2 polypeptides, IPD097 polypeptides, IPD099-1 polypeptides, IPD099-2 polypeptides, IPD099-3 polypeptides, IPD100-1 polypeptides, IPD100-2 polypeptides, IPD105 polypeptides, IPD106-1 polypeptides, IPD106-2 polypeptides, IPD107 polypeptides, IPD111 polypeptides or IPD112 polypeptides are encompassed.

In some embodiments, chimeric IPD092-1 polypeptides, chimeric IPD092-2 polypeptides, chimeric IPD095-1 polypeptides, chimeric IPD095-2 polypeptides, chimeric IPD097 polypeptides, chimeric IPD099-1 polypeptides, chimeric IPD099-2 polypeptides, chimeric IPD099-3 polypeptides, chimeric IPD100-1 polypeptides, chimeric IPD100-2 polypeptides, chimeric IPD105 polypeptides, chimeric IPD106-1 polypeptides, chimeric IPD106-2 polypeptides, chimeric IPD107 polypeptides, chimeric IPD111 polypeptides or chimeric IPD112 polypeptides are provided comprising an N-terminal Region of a first IPD092-1 polypeptides, IPD092-2 polypeptides, IPD095-1 polypeptides, IPD095-2 polypeptides, IPD097 polypeptides, IPD099-1 polypeptides, IPD099-2 polypeptides, IPD099-3 polypeptides, IPD100-1 polypeptides, IPD100-2 polypeptides, IPD105 polypeptides, IPD106-1 polypeptides, IPD106-2 polypeptides, IPD107 polypeptides, IPD111 polypeptides or IPD112 polypeptides of the disclosure operably fused to a C-terminal Region of a second IPD092-1 polypeptides, IPD092-2 polypeptides, IPD095-1 polypeptides, IPD095-2 polypeptides, IPD097 polypeptides, IPD099-1 polypeptides, IPD099-2 polypeptides, IPD099-3 polypeptides, IPD100-1 polypeptides, IPD100-2 polypeptides, IPD105 polypeptides, IPD106-1 polypeptides, IPD106-2 polypeptides, IPD107 polypeptides, IPD111 polypeptides or IPD112 polypeptides of the disclosure.

In other embodiments, the polypeptides of the disclosure can be expressed as a precursor protein with an intervening sequence that catalyzes multi-step, post translational protein splicing. Protein splicing involves the excision of an intervening sequence from a polypeptide with the concomitant joining of the flanking sequences to yield a new polypeptide (Chong, et al., (1996) J. Biol. Chem., 271:22159-22168). This intervening sequence or protein splicing element, referred to as inteins, which catalyze their own excision through three coordinated reactions at the N-terminal and C-terminal splice junctions: an acyl rearrangement of the N-terminal cysteine or serine; a transesterfication reaction between the two termini to form a branched ester or thioester intermediate and peptide bond cleavage coupled to cyclization of the intein C-terminal asparagine to free the intein (Evans, et al., (2000) J. Biol. Chem., 275:9091-9094. In other embodiments, the polypeptides of the disclosure can be encoded by two separate genes where the intein of the precursor protein comes from the two genes, referred to as a split-intein, and the two portions of the precursor are joined by a peptide bond formation.

In some embodiments, the polypeptide of the disclosure is a circular permuted variant. The development of recombinant DNA methods has made it possible to study the effects of sequence transposition on protein folding, structure and function. The approach used in creating new sequences resembles that of naturally occurring pairs of proteins that are related by linear reorganization of their amino acid sequences (Cunningham, et al., (1979) Proc. Natl. Acad. Sci. U.S.A. 76:3218-3222; Teather and Erfle, (1990) J. Bacteriol. 172:3837-3841; Schimming, et al., (1992) Eur. J. Biochem. 204:13-19; Yamiuchi and Minamikawa, (1991) FEBS Lett. 260:127-130; MacGregor, et al., (1996) FEBS Lett. 378:263-266).

In another embodiment, fusion proteins are provided including within its amino acid sequence an amino acid sequence comprising a polypeptide of the disclosure. Polynucleotides encoding a polypeptide of the disclosure can be fused to a signal sequence transit peptide which will direct the localization of the polypeptide to particular compartments of a prokaryotic or eukaryotic cell and/or direct the secretion of the polypeptide of the embodiments from a prokaryotic or eukaryotic cell. For example, in E. coli, one may wish to direct the expression of the protein to the periplasmic space. Examples of signal sequences or proteins (or fragments thereof) to which the polypeptide may be fused to direct the expression of the polypeptide to the periplasmic space of bacteria include, but are not limited to, the pelB signal sequence, the maltose binding protein (MBP) signal sequence, MBP, the ompA signal sequence, the signal sequence of the periplasmic E. coli heat-labile enterotoxin B-subunit and the signal sequence of alkaline phosphatase. Several vectors are commercially available for the construction of fusion proteins which will direct the localization of a protein, such as the pMAL series of vectors (particularly the pMAL-p series) available from New England Biolabs. In a specific embodiment, the polypeptide of the disclosure may be fused to the pelB pectate lyase signal sequence to increase the efficiency of expression and purification of such polypeptides in Gram-negative bacteria (see, U.S. Pat. Nos. 5,576,195 and 5,846,818). Polynucleotides encoding a polypeptide of the disclosure can be fused to a plant plastid transit peptide. The plastid transit peptide is generally fused N-terminal to the polypeptide to be targeted (e.g., the fusion partner). In one embodiment, the fusion protein consists essentially of the plastid transit peptide and the polypeptide of the disclosure to be targeted. In another embodiment, the fusion protein comprises the plastid transit peptide and the polypeptide to be targeted. In such embodiments, the plastid transit peptide is preferably at the N-terminus of the fusion protein. However, additional amino acid residues may be N-terminal to the plastid transit peptide if the fusion protein is at least partially targeted to a plastid. In a specific embodiment, the plastid transit peptide is in the N-terminal half, N-terminal third or N-terminal quarter of the fusion protein. Most or all the plastid transit peptide is generally cleaved from the fusion protein upon insertion into the plastid. The position of cleavage may vary slightly between plant species, at different plant developmental stages, because of specific intercellular conditions or the combination of transit peptide/fusion partner used. In one embodiment, the plastid transit peptide cleavage is homogenous such that the cleavage site is identical in a population of fusion proteins. In another embodiment, the plastid transit peptide is not homogenous, such that the cleavage site varies by 1-10 amino acids in a population of fusion proteins. The plastid transit peptide can be recombinantly fused to a second protein in one of several ways. For example, a restriction endonuclease recognition site can be introduced into the nucleotide sequence of the transit peptide at a position corresponding to its C-terminal end and the same or a compatible site can be engineered into the nucleotide sequence of the protein to be targeted at its N-terminal end. Care must be taken in designing these sites to ensure that the coding sequences of the transit peptide and the second protein are kept “in frame” to allow the synthesis of the desired fusion protein. In some cases, it may be preferable to remove the initiator methionine of the second protein when the new restriction site is introduced. The introduction of restriction endonuclease recognition sites on both parent molecules and their subsequent joining through recombinant DNA techniques may result in the addition of one or more extra amino acids between the transit peptide and the second protein. This generally does not affect targeting activity if the transit peptide cleavage site remains accessible and the function of the second protein is not altered by the addition of these extra amino acids at its N-terminus. Alternatively, one skilled in the art can create a precise cleavage site between the transit peptide and the second protein (with or without its initiator methionine) using gene synthesis (Stemmer, et al., (1995) Gene 164:49-53) or similar methods. In addition, the transit peptide fusion can intentionally include amino acids downstream of the cleavage site. The amino acids at the N-terminus of the mature protein can affect the ability of the transit peptide to target proteins to plastids and/or the efficiency of cleavage following protein import. This may be dependent on the protein to be targeted. See, e.g., Comai, et al., (1988) J. Biol. Chem. 263(29):15104-9. In some embodiments, the polypeptide of the disclosure is fused to a heterologous signal peptide or heterologous transit peptide.

### Nucleic Acid Molecules, and Variants and Fragments Thereof

Isolated or recombinant nucleic acid molecules comprising nucleic acid sequences encoding polypeptides of the disclosure or biologically active portions thereof, as well as nucleic acid molecules sufficient for use as hybridization probes to identify nucleic acid molecules encoding proteins with regions of sequence homology are provided. As used herein, the term “nucleic acid molecule” refers to DNA molecules (e.g., recombinant DNA, cDNA, genomic DNA, plastid DNA, mitochondrial DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule (or DNA) is used herein to refer to a nucleic acid sequence (or DNA) that is no longer in its natural environment, for example in vitro. A “recombinant” nucleic acid molecule (or DNA) is used herein to refer to a nucleic acid sequence (or DNA) that is in a recombinant bacterial or plant host cell. In some embodiments, an “isolated” or “recombinant” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For purposes of the disclosure, “isolated” or “recombinant” when used to refer to nucleic acid molecules excludes isolated chromosomes. For example, in various embodiments, the recombinant nucleic acid molecules encoding polypeptides of the disclosure can contain less than about 5 kb, 4 kb, 3 kb, 2 kb, 1 kb, 0.5 kb or 0.1 kb of nucleic acid sequences that naturally flank the nucleic acid molecule in genomic DNA of the cell from which the nucleic acid is derived.

In some embodiments, an isolated nucleic acid molecule encoding polypeptides of the disclosure has one or more change in the nucleic acid sequence compared to the native or genomic nucleic acid sequence. In some embodiments, the change in the native or genomic nucleic acid sequence includes but is not limited to: changes in the nucleic acid sequence due to the degeneracy of the genetic code; changes in the nucleic acid sequence due to the amino acid substitution, insertion, deletion and/or addition compared to the native or genomic sequence; removal of one or more intron; deletion of one or more upstream or downstream regulatory regions; and deletion of the 5′ and/or 3′ untranslated region associated with the genomic nucleic acid sequence. In some embodiments, the nucleic acid molecule encoding a polypeptide of the disclosure is a non-genomic sequence.

A variety of polynucleotides encoding polypeptides of the disclosure or related proteins are contemplated. Such polynucleotides are useful for production of polypeptides of the disclosure in host cells when operably linked to a suitable promoter, transcription termination and/or polyadenylation sequences. Such polynucleotides are also useful as probes for isolating homologous or substantially homologous polynucleotides encoding polypeptides of the disclosure or related proteins.

### Polynucleotides Encoding IPD092-1 Polypeptides

Sources of polynucleotides encoding IPD092-1 polypeptides or related proteins are a Pseudomonas or Woodsholea bacterium.

In some embodiments polynucleotides are provided encoding an IPD092-1 polypeptide comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 22, SEQ ID NO: 24 or SEQ ID NO: 26.

In some embodiments, the polynucleotide of the disclosure encodes an IPD092-1 polypeptide comprising an amino acid sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 22, SEQ ID NO: 24 or SEQ ID NO: 26 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70,71, 72, 73, 74, 75, 76, 77, 78, 79, 80, 81, 82, 83, 84, 85, 86, 87, 88, 89, 90, 91, 92, 93, 94, 95 or more amino acid substitutions compared to the native amino acid at the corresponding position of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 22, SEQ ID NO: 24 or SEQ ID NO: 26.

In some embodiments, the nucleic acid molecule encoding an IPD092-1 polypeptide, comprises the sequence set forth in SEQ ID NO: 546, SEQ ID NO: 549, SEQ ID NO: 550, SEQ ID NO: 552, SEQ ID NO: 554, SEQ ID NO: 556, SEQ ID NO: 558 or SEQ ID NO: 560, and variants, fragments and complements thereof. “Polynucleotide sequence variants” is used herein to refer to a nucleic acid sequence that except for the degeneracy of the genetic code encodes the same polypeptide.

### Polynucleotides Encoding IPD092-2 Polypeptides

Sources of polynucleotides encoding IPD092-2 polypeptide homologs or related proteins include bacterial species selected from but not limited to Pseudomonas species, Chromobacterium species, Burkholderia species, and Woodsholea species.

In some embodiments polynucleotides are provided encoding an IPD092-2 polypeptide having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 21, SEQ ID NO: 23 or SEQ ID NO: 25.

In some embodiments polynucleotides are provided encoding an IPD092-2 polypeptide comprising an amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 21, SEQ ID NO: 23 or SEQ ID NO: 25 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more amino acid substitutions compared to the native amino acid at the corresponding position of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 21, SEQ ID NO: 23 or SEQ ID NO: 25.

In some embodiments polynucleotides are provided encoding an IPD092-2 polypeptide comprising the amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 21, SEQ ID NO: 23 or SEQ ID NO: 25.

In some embodiments, the nucleic acid molecule encoding an IPD092-2 polypeptide, comprises the sequence set forth in SEQ ID NO: 547, SEQ ID NO: 548, SEQ ID NO: 551, SEQ ID NO: 553, SEQ ID NO: 555, SEQ ID NO: 557, SEQ ID NO: 559 or SEQ ID NO: 561, and variants, fragments and complements thereof.

### Polynucleotides Encoding IPD095-1 Polypeptides

Sources of polynucleotides encoding IPD095-1 polypeptide homologs or related proteins include bacterial species selected from but not limited to Serratia species, Leminorella species, Dickeya species, Enterobacter species, Erwinia species, Yersinia species, and Rahnella species.

In some embodiments polynucleotides are provided encoding an IPD095-1 polypeptide comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 27, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 or SEQ ID NO: 58.

In some embodiments polynucleotides of are provided encoding an IPD095-1 polypeptide comprising an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 27, SEQ ID NO: 29, SEQ ID NO: 30, SEQ ID NO: 34, SEQ ID NO: 35, SEQ ID NO: 38, SEQ ID NO: 41, SEQ ID NO: 42, SEQ ID NO: 43, SEQ ID NO: 48, SEQ ID NO: 49, SEQ ID NO: 50, SEQ ID NO: 51, SEQ ID NO: 54, SEQ ID NO: 56 or SEQ ID NO: 57.

In some embodiments polynucleotides are provided encoding an IPD095-1 polypeptide comprising an amino acid sequence of SEQ ID NO: 27, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 or SEQ ID NO: 58 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85 or more amino acid substitutions compared to the native amino acid at the corresponding position of SEQ ID NO: 27, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 or SEQ ID NO: 58.

In some embodiments polynucleotides are provided encoding an IPD095-1 polypeptide comprising the amino acid sequence of SEQ ID NO: 27, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 or SEQ ID NO: 58.

In some embodiments polynucleotides are provided encoding an IPD095-1 polypeptide comprising the amino acid sequence of SEQ ID NO: 27, SEQ ID NO: 29, SEQ ID NO: 30, SEQ ID NO: 34, SEQ ID NO: 35, SEQ ID NO: 38, SEQ ID NO: 41, SEQ ID NO: 42, SEQ ID NO: 43, SEQ ID NO: 48, SEQ ID NO: 49, SEQ ID NO: 50, SEQ ID NO: 51, SEQ ID NO: 54, SEQ ID NO: 56 or SEQ ID NO: 57.

In some embodiments polynucleotides are provided, encoding an IPD095-1 polypeptide, comprising the nucleic acid sequence of SEQ ID NO: 562, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573 or SEQ ID NO: 574.

### Polynucleotides Encoding IPD095-2 Polypeptides

Sources of polynucleotides encoding an IPD095-2 polypeptide homologs or related proteins include bacterial species selected from but not limited to Serratia species, Leminorella species, Dickeya species, Enterobacter species, Erwinia species, Yersinia species, and Rahnella species. In some embodiments, the polynucleotide of the disclosure encodes an IPD095-2 polypeptide comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 28, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119 or SEQ ID NO: 120.

In some embodiments, the polynucleotide of the disclosure encodes an IPD095-2 polypeptide comprising an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 28, SEQ ID NO: 59, SEQ ID NO: 60, SEQ ID NO: 61, SEQ ID NO: 63, SEQ ID NO: 66, SEQ ID NO: 67, SEQ ID NO: 68, SEQ ID NO: 75, SEQ ID NO: 77, SEQ ID NO: 78, SEQ ID NO: 79, SEQ ID NO: 82, SEQ ID NO: 83, SEQ ID NO: 84, SEQ ID NO: 85, SEQ ID NO: 86, SEQ ID NO: 94, SEQ ID NO: 95, SEQ ID NO: 96, SEQ ID NO: 97, SEQ ID NO: 99, SEQ ID NO: 100, SEQ ID NO: 103, SEQ ID NO: 107, SEQ ID NO: 119 or SEQ ID NO: 120.

In some embodiments polynucleotides are provided encoding an IPD095-2 polypeptide comprising an amino acid sequence of SEQ ID NO: 28, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119 or SEQ ID NO: 120 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85 or more amino acid substitutions compared to the native amino acid at the corresponding position of SEQ ID NO: 28, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119 or SEQ ID NO: 120.

In some embodiments, the polynucleotide of the disclosure encodes an IPD095-2 polypeptide comprising the amino acid sequence of SEQ ID NO: 28, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119 or SEQ ID NO: 120.

In some embodiments polynucleotides are provided encoding an IPD095-2 polypeptide comprising the amino acid sequence of SEQ ID NO: 28, SEQ ID NO: 59, SEQ ID NO: 60, SEQ ID NO: 61, SEQ ID NO: 63, SEQ ID NO: 66, SEQ ID NO: 67, SEQ ID NO: 68, SEQ ID NO: 75, SEQ ID NO: 77, SEQ ID NO: 78, SEQ ID NO: 79, SEQ ID NO: 82, SEQ ID NO: 83, SEQ ID NO: 84, SEQ ID NO: 85, SEQ ID NO: 86, SEQ ID NO: 94, SEQ ID NO: 95, SEQ ID NO: 96, SEQ ID NO: 97, SEQ ID NO: 99, SEQ ID NO: 100, SEQ ID NO: 103, SEQ ID NO: 107, SEQ ID NO: 119 or SEQ ID NO: 120.

In some embodiments polynucleotides are provided, encoding an IPD095-2 polypeptide, comprising the nucleic acid sequence of SEQ ID NO: 563, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, SEQ ID NO: 588 or SEQ ID NO: 589.

### Polynucleotides Encoding IPD097 Polypeptides

Sources of polynucleotides encoding IPD097 polypeptide homologs or related proteins include bacterial species selected from but not limited to Haemophilus species, Aeromonas species, and Clostridiales species. In some embodiments polynucleotides are provided encoding an IPD097 polypeptide comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134 or SEQ ID NO: 135.

In some embodiments polynucleotides are provided encoding an IPD097 polypeptide comprising an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 121, SEQ ID NO: 123, SEQ ID NO: 127, SEQ ID NO: 128, SEQ ID NO: 129, SEQ ID NO: 131 or SEQ ID NO: 132.

In some embodiments polynucleotides are provided encoding an IPD097 polypeptide comprising an amino acid sequence of SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134 or SEQ ID NO: 135 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more amino acid substitutions compared to the native amino acid at the corresponding position of SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134 or SEQ ID NO: 135.

In some embodiments polynucleotides are provided encoding an IPD097 polypeptide comprising the amino acid sequence of SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134 or SEQ ID NO: 135.

In some embodiments polynucleotides are provided encoding an IPD097 polypeptide comprising the amino acid sequence of SEQ ID NO: 121, SEQ ID NO: 123, SEQ ID NO: 127, SEQ ID NO: 128, SEQ ID NO: 129, SEQ ID NO: 131 or SEQ ID NO: 132.

In some embodiments polynucleotides are provided, encoding an IPD097 polypeptide, comprising the nucleic acid sequence of SEQ ID NO: 590.

### Polynucleotides Encoding IPD099-1 Polypeptides

Sources of polynucleotides encoding IPD099-1 polypeptide homologs or related proteins include bacterial species selected from but not limited to Aeromonas species, Haemophilus species, Burkholderia species, Chromobacterium species, Erwinia species, Serratia species, Salinivibrio species, Aquimarina species, Janthinobacterium species, Tolypothrix species, Photobacterium species, Janthinobacterium species, Rhizobium species, Moritella species, Providencia species, Yersinia species and Vibrio species.

In some embodiments polynucleotides are provided encoding an IPD099-1 polypeptide comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 136, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214 or SEQ ID NO: 215.

In some embodiments polynucleotides are provided encoding an IPD099-1 polypeptide comprising an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 136, SEQ ID NO: 139, SEQ ID NO: 140, SEQ ID NO: 142, SEQ ID NO: 144, SEQ ID NO: 145, SEQ ID NO: 148, SEQ ID NO: 149, SEQ ID NO: 150, SEQ ID NO: 155, SEQ ID NO: 157, SEQ ID NO: 158, SEQ ID NO: 177, SEQ ID NO: 179, SEQ ID NO: 180, SEQ ID NO: 183, SEQ ID NO: 185, SEQ ID NO: 186, SEQ ID NO: 187, SEQ ID NO: 188, SEQ ID NO: 189, SEQ ID NO: 192, SEQ ID NO: 195, SEQ ID NO: 196, SEQ ID NO: 197, SEQ ID NO: 199, SEQ ID NO: 205, SEQ ID NO: 208, SEQ ID NO: 209, SEQ ID NO: 210 or SEQ ID NO: 215.

In some embodiments polynucleotides are provided encoding an IPD099-1 polypeptide comprising an amino acid sequence of SEQ ID NO: 136, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214 or SEQ ID NO: 215 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more amino acid substitutions compared to the native amino acid at the corresponding position of SEQ ID NO: 136, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214 or SEQ ID NO: 215.

In some embodiments polynucleotides are provided encoding an IPD099-1 polypeptide comprising the amino acid sequence of SEQ ID NO: 136, SEQ ID NO: 139, SEQ ID NO: 140, SEQ ID NO: 141, SEQ ID NO: 142, SEQ ID NO: 143, SEQ ID NO: 144, SEQ ID NO: 145, SEQ

ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214 or SEQ ID NO: 215.

In some embodiments polynucleotides are provided encoding an IPD099-1 polypeptide comprising the amino acid sequence of SEQ ID NO: 136, SEQ ID NO: 139, SEQ ID NO: 140, SEQ ID NO: 142, SEQ ID NO: 144, SEQ ID NO: 145, SEQ ID NO: 148, SEQ ID NO: 149, SEQ ID NO: 150, SEQ ID NO: 155, SEQ ID NO: 157, SEQ ID NO: 158, SEQ ID NO: 177, SEQ ID NO: 179, SEQ ID NO: 180, SEQ ID NO: 183, SEQ ID NO: 185, SEQ ID NO: 186, SEQ ID NO: 187, SEQ ID NO: 188, SEQ ID NO: 189, SEQ ID NO: 192, SEQ ID NO: 195, SEQ ID NO: 196, SEQ ID NO: 197, SEQ ID NO: 199, SEQ ID NO: 205, SEQ ID NO: 208, SEQ ID NO: 209, SEQ ID NO: 210 or SEQ ID NO: 215.

In some embodiments polynucleotides are provided, encoding an IPD099-1 polypeptide, comprising the nucleic acid sequence of SEQ ID NO: 591, SEQ ID NO: 594, SEQ ID NO: 595, SEQ ID NO: 596, SEQ ID NO: 597, SEQ ID NO: 598, SEQ ID NO: 599, SEQ ID NO: 600, SEQ ID NO: 601, SEQ ID NO: 602.

### Polynucleotides Encoding IPD099-2 Polypeptides

Sources of polynucleotides encoding IPD099-2 polypeptide homologs or related proteins include bacterial species selected from but not limited to Aeromonas species, Haemophilus species, Burkholderia species, Chromobacterium species, Erwinia species, Serratia species, Salinivibrio species, Aquimarina species, Janthinobacterium species, Tolypothrix species, Photobacterium species, Janthinobacterium species, Rhizobium species, Moritella species, Providencia species, Yersinia species and Vibrio species.

In some embodiments polynucleotides are provided encoding an IPD099-2 polypeptide having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 137, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270 or SEQ ID NO: 271.

In some embodiments polynucleotides are provided encoding an IPD099-2 polypeptide comprising an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 137, SEQ ID NO: 216, SEQ ID NO: 221, SEQ ID NO: 222, SEQ ID NO: 225, SEQ ID NO: 227, SEQ ID NO: 230, SEQ ID NO: 232, SEQ ID NO: 244, SEQ ID NO: 246, SEQ ID NO: 249, SEQ ID NO: 251, SEQ ID NO: 254, SEQ ID NO: 255, SEQ ID NO: 262, SEQ ID NO: 265, SEQ ID NO: 268 or SEQ ID NO: 269.

In some embodiments polynucleotides are provided encoding an IPD099-2 polypeptide comprising an amino acid sequence of SEQ ID NO: 137, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270 or SEQ ID NO: 271 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more amino acid substitutions compared to the native amino acid at the corresponding position of SEQ ID NO: 137, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270 or SEQ ID NO: 271.

In some embodiments polynucleotides are provided encoding an IPD099-2 polypeptide comprising the amino acid sequence of SEQ ID NO: 137, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270 or SEQ ID NO: 271.

In some embodiments polynucleotides are provided encoding an IPD099-2 polypeptide comprising the amino acid sequence of SEQ ID NO: 137, SEQ ID NO: 216, SEQ ID NO: 221, SEQ ID NO: 222, SEQ ID NO: 225, SEQ ID NO: 227, SEQ ID NO: 230, SEQ ID NO: 232, SEQ ID NO: 244, SEQ ID NO: 246, SEQ ID NO: 249, SEQ ID NO: 251, SEQ ID NO: 254, SEQ ID NO: 255, SEQ ID NO: 262, SEQ ID NO: 265, SEQ ID NO: 268 or SEQ ID NO: 269.

In some embodiments polynucleotides are provided, encoding an IPD099-2 polypeptide, comprising the nucleic acid sequence of SEQ ID NO: 592, SEQ ID NO: 603 or SEQ ID NO: 605.

### Polynucleotides Encoding IPD099-3 Ppolypeptides

Sources of polynucleotides encoding IPD099-3 polypeptide homologs or related proteins include bacterial species selected from but not limited to Aeromonas species, Haemophilus species, Burkholderia species, Chromobacterium species, Erwinia species, Serratia species, Salinivibrio species, Aquimarina species, Janthinobacterium species, Tolypothrix species, Photobacterium species, Janthinobacterium species, Rhizobium species, Moritella species, Providencia species, Yersinia species and Vibrio species.

In some embodiments polynucleotides are provide encoding an IPD099-3 polypeptide comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 138, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330 or SEQ ID NO: 331.

In some embodiments polynucleotides are provided encoding an IPD099-3 polypeptide comprising an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 138, SEQ ID NO: 272, SEQ ID NO: 273, SEQ ID NO: 275, SEQ ID NO: 285, SEQ ID NO: 286, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 298, SEQ ID NO: 299, SEQ ID NO: 300, SEQ ID NO: 301, SEQ ID NO: 302, SEQ ID NO: 304, SEQ ID NO: 306, SEQ ID NO: 307, SEQ ID NO: 308, SEQ ID NO: 312, SEQ ID NO: 313, SEQ ID NO: 320, SEQ ID NO: 323, SEQ ID NO: 324, SEQ ID NO: 326 or SEQ ID NO: 331.

In some embodiments polynucleotides are provided encoding an IPD099-3 polypeptide comprising an amino acid sequence of SEQ ID NO: 138, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330 or SEQ ID NO: 331 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more amino acid substitutions compared to the native amino acid at the corresponding position of SEQ ID NO: 138, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330 or SEQ ID NO: 331.

In some embodiments polynucleotides are provided the encode an IPD099-3 polypeptide comprising the amino acid sequence of SEQ ID NO: 138, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330 or SEQ ID NO: 331.

In some embodiments polynucleotides are provided encoding an IPD099-3 polypeptide comprising the amino acid sequence of SEQ ID NO: 138, SEQ ID NO: 272, SEQ ID NO: 273, SEQ ID NO: 275, SEQ ID NO: 285, SEQ ID NO: 286, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 298, SEQ ID NO: 299, SEQ ID NO: 300, SEQ ID NO: 301, SEQ ID NO: 302, SEQ ID NO: 304, SEQ ID NO: 306, SEQ ID NO: 307, SEQ ID NO: 308, SEQ ID NO: 312, SEQ ID NO: 313, SEQ ID NO: 320, SEQ ID NO: 323, SEQ ID NO: 324, SEQ ID NO: 326, SEQ ID NO: 331.

In some embodiments polynucleotides are provided, encoding an IPD099-3 polypeptide, comprising the nucleic acid sequence of SEQ ID NO: 593, SEQ ID NO: 605, SEQ ID NO: 606, SEQ ID NO: 607, SEQ ID NO: 608, SEQ ID NO; 609 or SEQ ID NO: 610.

### Polynucleotides Encoding IPD100-1 Polypeptides

Sources of polynucleotides encoding IPD100-1 polypeptide homologs or related proteins include bacterial species selected from but not limited to Pseudomonas species, Candidatus species, Burkholderia species, Duganella species, Salmonella species, Tenacibaculum species, Dickeya species, Pedobacter species, and Mycobacterium species.

In some embodiments polynucleotides are provided encoding an IPD100-1 polypeptide having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 332, SEQ ID NO: 334, SEQ ID NO: 335 or SEQ ID NO: 336.

In some embodiments polynucleotide are provided encoding an IPD100-1 polypeptide comprising an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 332.

In some embodiments polynucleotides are provided encoding an IPD100-1 polypeptide comprising an amino acid sequence of SEQ ID NO: 332, SEQ ID NO: 334, SEQ ID NO: 335 or SEQ ID NO: 336 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75 or more amino acid substitutions compared to the native amino acid at the corresponding position of SEQ ID NO: 332, SEQ ID NO: 334, SEQ ID NO: 335 or SEQ ID NO: 336.

In some embodiments polynucleotides are provided encoding an IPD100-1 polypeptide comprising the amino acid sequence of SEQ ID NO: 332, SEQ ID NO: 334, SEQ ID NO: 335 or SEQ ID NO: 336.

In some embodiments polynucleotides are provided encoding an IPD100-1 polypeptide comprising the amino acid sequence of SEQ ID NO: 332.

In some embodiments polynucleotides are provided, encoding an IPD100-1 polypeptide, comprising the nucleic acid sequence of SEQ ID NO: 611.

### Polynucleotides Encoding IPD100-2 Polypeptides

Sources of polynucleotides encoding IPD100-2 polypeptide homologs or related proteins include bacterial species selected from but not limited to Pseudomonas species, Candidatus species, Burkholderia species, Duganella species, Salmonella species, Tenacibaculum species, Dickeya species, Pedobacter species, and Mycobacterium species.

In some embodiments polynucleotides are provided encoding an IPD100-2 polypeptide comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 333, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348 or SEQ ID NO: 349.

In some embodiments polynucleotides are provided encoding an IPD100-2 polypeptide comprising an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 333, SEQ ID NO: 337, SEQ ID NO: 338, SEQ ID NO: 341, SEQ ID NO: 342, SEQ ID NO: 343, SEQ ID NO: 344 or SEQ ID NO: 347.

In some embodiments polynucleotides are provided encoding an IPD100-2 polypeptide comprising an amino acid sequence of SEQ ID NO: 333, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348 or SEQ ID NO: 349 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more amino acid substitutions compared to the native amino acid at the corresponding position of SEQ ID NO: 333, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348 or SEQ ID NO: 349.

In some embodiments polynucleotides are provided encoding an IPD100-2 polypeptide comprising the amino acid sequence of SEQ ID NO: 333, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348 or SEQ ID NO: 349.

In some embodiments polynucleotides are provided encoding an IPD100-2 polypeptide comprising the amino acid sequence of SEQ ID NO: 333, SEQ ID NO: 337, SEQ ID NO: 338, SEQ ID NO: 341, SEQ ID NO: 342, SEQ ID NO: 343, SEQ ID NO: 344 or SEQ ID NO: 347.

In some embodiments polynucleotides are provided, encoding an IPD100-2 polypeptide, comprising the nucleic acid sequence of SEQ ID NO: 612 or SEQ ID NO: 613.

### Polynucleotides Encoding IPD105 Polypeptides

Sources of polynucleotides encoding IPD105 polypeptide homologs or related proteins include bacterial species selected from but not limited to Chromobacterium species and Pseudogulbenkiania l species.

In some embodiments polynucleotides are provided encoding an IPD105 polypeptide having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364 or SEQ ID NO: 365.

In some embodiments polynucleotides are provided encoding an IPD105 polypeptide comprising an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 350, SEQ ID NO: 353, SEQ ID NO: 355, SEQ ID NO: 357 or SEQ ID NO: 362.

In some embodiments polynucleotides are provided encoding an IPD105 polypeptide comprising an amino acid sequence of SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364 or SEQ ID NO: 365 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more amino acid substitutions compared to the native amino acid at the corresponding position of SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364 or SEQ ID NO: 365.

In some embodiments polynucleotides are provided encoding an IPD105 polypeptide comprising the amino acid sequence of SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364 or SEQ ID NO: 365.

In some embodiments polynucleotides are provided encoding an IPD105 polypeptide comprising the amino acid sequence of SEQ ID NO: 350, SEQ ID NO: 353, SEQ ID NO: 355, SEQ ID NO: 357 or SEQ ID NO: 362.

In some embodiments polynucleotides are provided, encoding an IPD105 polypeptide, comprising the nucleic acid sequence of SEQ ID NO: 614, SEQ ID NO: 615 or SEQ ID NO: 616.

### Polynucleotides Encoding IPD106-1 Polypeptides

Sources of polynucleotides encoding IPD106-1 polypeptide homologs or related proteins include bacterial species selected from but not limited to Arsenicibacter species and Chitinophaga species.

In some embodiments polynucleotides are provided encoding an IPD106-1 polypeptide having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 366, SEQ ID NO: 368, SEQ ID NO: 369, SEQ ID NO: 370 or SEQ ID NO: 371.

In some embodiments polynucleotides are provided encoding an IPD106-1 polypeptide comprising an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 366, SEQ ID NO: 368 or SEQ ID NO: 369.

In some embodiments polynucleotides are provided encoding an IPD106-1 polypeptide comprising an amino acid sequence of SEQ ID NO: 366, SEQ ID NO: 368, SEQ ID NO: 369, SEQ ID NO: 370 or SEQ ID NO: 371 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more amino acid substitutions compared to the native amino acid at the corresponding position of SEQ ID NO: 366, SEQ ID NO: 368, SEQ ID NO: 369, SEQ ID NO: 370 or SEQ ID NO: 371.

In some embodiments polynucleotides are provided the encode an IPD106-1 polypeptide comprising the amino acid sequence of SEQ ID NO: 366, SEQ ID NO: 368, SEQ ID NO: 369, SEQ ID NO: 370 or SEQ ID NO: 371.

In some embodiments polynucleotides are provided encoding an IPD106-1 polypeptide comprising the amino acid sequence of SEQ ID NO: 366, SEQ ID NO: 368 or SEQ ID NO: 369.

In some embodiments polynucleotides are provided, encoding an IPD105 polypeptide, comprising the nucleic acid sequence of SEQ ID NO: 617 or SEQ ID NO: 619.

### Polynucleotides Encoding IPD106-2 Polypeptides

Sources of polynucleotides encoding IPD106-2 polypeptide homologs or related proteins include bacterial species selected from but not limited to Arsenicibacter species and Chitinophaga species.

In some embodiments polynucleotides are provided encoding an IPD106-2 polypeptide comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 367, SEQ ID NO: 372, SEQ ID NO: 373, SEQ ID NO: 374, SEQ ID NO: 375 or SEQ ID NO: 376.

In some embodiments polynucleotides are provide encoding an IPD106-2 polypeptide comprising an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 367, SEQ ID NO: 372, SEQ ID NO: 373 or SEQ ID NO: 376.

In some embodiments polynucleotides are provided encoding an IPD106-2 polypeptide comprising an amino acid sequence of SEQ ID NO: 367, SEQ ID NO: 372, SEQ ID NO: 373, SEQ ID NO: 374, SEQ ID NO: 375 or SEQ ID NO: 376 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more amino acid substitutions compared to the native amino acid at the corresponding position of SEQ ID NO: 367, SEQ ID NO: 372, SEQ ID NO: 373, SEQ ID NO: 374, SEQ ID NO: 375 or SEQ ID NO: 376.

In some embodiments polynucleotides are provided encoding an IPD106-2 polypeptide comprising the amino acid sequence of SEQ ID NO: 367, SEQ ID NO: 372, SEQ ID NO: 373, SEQ ID NO: 374, SEQ ID NO: 375 or SEQ ID NO: 376.

In some embodiments polynucleotides are provided encoding an IPD106-2 polypeptide comprising the amino acid sequence of SEQ ID NO: 367, SEQ ID NO: 372, SEQ ID NO: 373 or SEQ ID NO: 376.

In some embodiments polynucleotides are provided, encoding an IPD106-2 polypeptide, comprising the nucleic acid sequence of SEQ ID NO: 618 or SEQ ID NO: 620.

### Polynucleotides Encoding IPD107 Polypeptides

Sources of polynucleotides encoding IPD107 polypeptide homologs or related proteins include bacterial species selected from but not limited to Pseudomonas species, Chromobacterium species, and Bradyrhizobium species.

In some embodiments polynucleotides are provided encoding an IPD107 polypeptide comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451 or SEQ ID NO: 452.

In some embodiments polynucleotides are provided encoding an IPD107 polypeptide comprising an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 377, SEQ ID NO: 378, SEQ ID NO: 381, SEQ ID NO: 382, SEQ ID NO: 384, SEQ ID NO: 386, SEQ ID NO: 387, SEQ ID NO: 388, SEQ ID NO: 389, SEQ ID NO: 390, SEQ ID NO: 391, SEQ ID NO: 393, SEQ ID NO: 396, SEQ ID NO: 397, SEQ ID NO: 398, SEQ ID NO: 400, SEQ ID NO: 401, SEQ ID NO: 402, SEQ ID NO: 403, SEQ ID NO: 404, SEQ ID NO: 405, SEQ ID NO: 407, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 417, SEQ ID NO: 418, SEQ ID NO: 419, SEQ ID NO: 420, SEQ ID NO: 421, SEQ ID NO: 422, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 438, SEQ ID NO: 439, SEQ ID NO: 440, SEQ ID NO: 442, SEQ ID NO: 443, SEQ ID NO: 445, SEQ ID NO: 446, SEQ ID NO: 451 or SEQ ID NO: 452.

In some embodiments polynucleotides are provide encoding an IPD107 polypeptide comprising an amino acid sequence of SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451 or SEQ ID NO: 452 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more amino acid substitutions compared to the native amino acid at the corresponding position of SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451 or SEQ ID NO: 452.

In some embodiments polynucleotides are provided encoding an IPD107 polypeptide comprising the amino acid sequence of SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451 or SEQ ID NO: 452.

In some embodiments polynucleotides are provided encoding an IPD107 polypeptide comprising the amino acid sequence of SEQ ID NO: 377, SEQ ID NO: 378, SEQ ID NO: 381, SEQ ID NO: 382, SEQ ID NO: 384, SEQ ID NO: 386, SEQ ID NO: 387, SEQ ID NO: 388, SEQ ID NO: 389, SEQ ID NO: 390, SEQ ID NO: 391, SEQ ID NO: 393, SEQ ID NO: 396, SEQ ID NO: 397, SEQ ID NO: 398, SEQ ID NO: 400, SEQ ID NO: 401, SEQ ID NO: 402, SEQ ID NO: 403, SEQ ID NO: 404, SEQ ID NO: 405, SEQ ID NO: 407, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 417, SEQ ID NO: 418, SEQ ID NO: 419, SEQ ID NO: 420, SEQ ID NO: 421, SEQ ID NO: 422, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 438, SEQ ID NO: 439, SEQ ID NO: 440, SEQ ID NO: 442, SEQ ID NO: 443, SEQ ID NO: 445, SEQ ID NO: 446, SEQ ID NO: 451 or SEQ ID NO: 452.

In some embodiments polynucleotides are provided, encoding an IPD107 polypeptide, comprising the nucleic acid sequence of SEQ ID NO: 621, SEQ ID NO: 622, SEQ ID NO: 623, SEQ ID NO: 624, SEQ ID NO: 625, SEQ ID NO: 626, SEQ ID NO: 627 or SEQ ID NO: 628

### Polynucleotides Encoding IPD111 Polypeptides

Sources of polynucleotides encoding IPD111 polypeptide homologs or related proteins include bacterial species selected from but not limited to Pseudomonas species, Chromobacterium species, and Burkholderia species

In some embodiments polynucleotides are provided encoding an IPD111 polypeptide comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 453, SEQ ID NO: 454, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524, SEQ ID NO: 525, SEQ ID NO: 526, SEQ ID NO: 527 or SEQ ID NO: 528.

In some embodiments polynucleotides are provided encoding an IPD111 polypeptide comprising an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 453, SEQ ID NO: 454, SEQ ID NO: 455, SEQ ID NO: 456, SEQ ID NO: 462, SEQ ID NO: 463, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 478, SEQ ID NO: 479, SEQ ID NO: 489, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524 or SEQ ID NO: 526.

In some embodiments polynucleotides are provided encoding an IPD111 polypeptide comprising an amino acid sequence of SEQ ID NO: 453, SEQ ID NO: 454, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524, SEQ ID NO: 525, SEQ ID NO: 526, SEQ ID NO: 527 or SEQ ID NO: 528 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more amino acid substitutions compared to the native amino acid at the corresponding position of SEQ ID NO: 453, SEQ ID NO: 454, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524, SEQ ID NO: 525, SEQ ID NO: 526, SEQ ID NO: 527 or SEQ ID NO: 528.

In some embodiments polynucleotides are provided encoding an IPD111 polypeptide comprising the amino acid sequence of SEQ ID NO: 453, SEQ ID NO: 454, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524, SEQ ID NO: 525, SEQ ID NO: 526, SEQ ID NO: 527 or SEQ ID NO: 528.

In some embodiments polynucleotides are provided encoding an IPD111 polypeptide comprising the amino acid sequence of SEQ ID NO: 453, SEQ ID NO: 454, SEQ ID NO: 455, SEQ ID NO: 456, SEQ ID NO: 462, SEQ ID NO: 463, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 478, SEQ ID NO: 479, SEQ ID NO: 489, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524 or SEQ ID NO: 526.

In some embodiments polynucleotides are provided, encoding an IPD111 polypeptide, comprising the nucleic acid sequence of SEQ ID NO: 629, SEQ ID NO: 630, SEQ ID NO: 631, SEQ ID NO: 632, SEQ ID NO: 633 or SEQ ID NO: 634.

### Polynucleotides Encoding IPD112 Polypeptides

Sources of IPD112 polypeptide homologs or related proteins include bacterial species selected from but not limited to Pseudomonas species and Hafnia species.

In some embodiments polynucleotides are provided encoding an IPD112 polypeptide comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544 or SEQ ID NO: 545.

In some embodiments polynucleotides are provided encoding an IPD112 polypeptide comprising an amino acid sequence having at least 95%, 95.5% 96%, 96.5%, 97%, 97%.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or greater identity across the entire length of the amino acid sequence of SEQ ID NO: 529, SEQ ID NO: 530, SEQ ID NO: 531, SEQ ID NO: 532, SEQ ID NO: 534, SEQ ID NO: 537 or SEQ ID NO: 545.

In some embodiments polynucleotides are provided encoding an IPD112 polypeptide comprising an amino acid sequence of SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544 or SEQ ID NO: 545 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more amino acid substitutions compared to the native amino acid at the corresponding position of SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544 or SEQ ID NO: 545.

In some embodiments polynucleotides are provided encoding an IPD112 polypeptide comprising the amino acid sequence of SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544 or SEQ ID NO: 545.

In some embodiments polynucleotides are provided encoding an IPD112 polypeptide comprising the amino acid sequence of SEQ ID NO: 529, SEQ ID NO: 530, SEQ ID NO: 531, SEQ ID NO: 532, SEQ ID NO: 534, SEQ ID NO: 537 or SEQ ID NO: 545.

In some embodiments polynucleotides are provided, encoding an IPD112 polypeptide, comprising the nucleic acid sequence of SEQ ID NO: 635, SEQ ID NO: 636, SEQ ID NO: 637 or SEQ ID NO: 638.

The polynucleotides of the disclosure can be used to express the disclosed polypeptides in recombinant bacterial hosts that include but are not limited to Agrobacterium, Bacillus, Escherichia, Salmonella, Pseudomonas and Rhizobium bacterial host cells. The polynucleotides are also useful as probes for isolating homologous or substantially homologous polynucleotides encoding the disclosed insecticidal polypeptides or related proteins. Such probes can be used to identify homologous or substantially homologous polynucleotides derived from Pseudomonas species.

Polynucleotides encoding the disclosed polypeptides can also be synthesized de novo from a disclosed polypeptide sequence. The sequence of the polynucleotide gene can be deduced from a disclosed polypeptide sequence through use of the genetic code. Computer programs such as “BackTranslate” (GCG™ Package, Acclerys, Inc. San Diego, Calif.) can be used to convert a peptide sequence to the corresponding nucleotide sequence encoding the peptide. Furthermore, synthetic polynucleotide sequences of the disclosure can be designed so that they will be expressed in plants.

In some embodiments, the nucleic acid molecule encoding a polypeptide of the disclosure is a non-genomic nucleic acid sequence. As used herein a “non-genomic nucleic acid sequence” or “non-genomic nucleic acid molecule” or “non-genomic polynucleotide” refers to a nucleic acid molecule that has one or more change in the nucleic acid sequence compared to a native or genomic nucleic acid sequence. In some embodiments, the change to a native or genomic nucleic acid molecule includes but is not limited to: changes in the nucleic acid sequence due to the degeneracy of the genetic code; optimization of the nucleic acid sequence for expression in plants; changes in the nucleic acid sequence to introduce at least one amino acid substitution, insertion, deletion and/or addition compared to the native or genomic sequence; removal of one or more intron associated with the genomic nucleic acid sequence; insertion of one or more heterologous introns; deletion of one or more upstream or downstream regulatory regions associated with the genomic nucleic acid sequence; insertion of one or more heterologous upstream or downstream regulatory regions; deletion of the 5′ and/or 3′ untranslated region associated with the genomic nucleic acid sequence; insertion of a heterologous 5′ and/or 3′ untranslated region; and modification of a polyadenylation site. In some embodiments, the non-genomic nucleic acid molecule is a synthetic nucleic acid sequence.

Also provided are nucleic acid molecules encoding transcription and/or translation products that are subsequently spliced to ultimately produce functional Polypeptides of the disclosure. Splicing can be accomplished in vitro or in vivo, and can involve cis- or trans-splicing. The substrate for splicing can be polynucleotides (e.g., RNA transcripts) or polypeptides. An example of cis-splicing of a polynucleotide is where an intron inserted into a coding sequence is removed and the two flanking exon regions are spliced to generate a polypeptide of the disclosure encoding sequence. An example of trans-splicing would be where a polynucleotide is encrypted by separating the coding sequence into two or more fragments that can be separately transcribed and then spliced to form the full-length pesticidal encoding sequence. The use of a splicing enhancer sequence, which can be introduced into a construct, can facilitate splicing either in cis or trans-splicing of polypeptides (U.S. Pat. Nos. 6,365,377 and 6,531,316). Thus, in some embodiments polynucleotides do not directly encode a full-length polypeptide of the disclosure, but rather encode a fragment or fragments of a polypeptide of the disclosure. These polynucleotides can be used to express a functional polypeptide of the disclosure through a mechanism involving splicing, where splicing can occur at the level of polynucleotide (e.g., intron/exon) and/or polypeptide (e.g., intein/extein). This can be useful, for example, in controlling expression of pesticidal activity, since a functional pesticidal polypeptide will only be expressed if all required fragments are expressed in an environment that permits splicing processes to generate functional product. In another example, introduction of one or more insertion sequences into a polynucleotide can facilitate recombination with a low homology polynucleotide; use of an intron or intein for the insertion sequence facilitates the removal of the intervening sequence, thereby restoring function of the encoded variant.

Nucleic acid molecules that are fragments of these nucleic acid sequences encoding Polypeptides of the disclosure are also encompassed by the embodiments. “Fragment” as used herein refers to a portion of the nucleic acid sequence encoding a polypeptide of the disclosure. A fragment of a nucleic acid sequence may encode a biologically active portion of a polypeptide of the disclosure or it may be a fragment that can be used as a hybridization probe or PCR primer using methods disclosed below. Nucleic acid molecules that are fragments of a nucleic acid sequence encoding a polypeptide of the disclosure comprise at least about 150, 180, 210, 240, 270, 300, 330 or 360, contiguous nucleotides or up to the number of nucleotides present in a full-length nucleic acid sequence encoding a polypeptide of the disclosure disclosed herein, depending upon the intended use. “Contiguous nucleotides” is used herein to refer to nucleotide residues that are immediately adjacent to one another. Fragments of the nucleic acid sequences of the embodiments will encode protein fragments that retain the biological activity of the polypeptide of the disclosure and, hence, retain insecticidal activity. “Retains insecticidal activity” is used herein to refer to a polypeptide having at least about 10%, at least about 30%, at least about 50%, at least about 70%, 80%, 90%, 95% or higher of the insecticidal activity of a full-length polypeptide of the disclosure. In some embodiments, the insecticidal activity is against a Lepidopteran species. In one embodiment, the insecticidal activity is against a Coleopteran species. In some embodiments, the insecticidal activity is against one or more insect pests of the corn rootworm complex: western corn rootworm, Diabrotica virgifera; northern corn rootworm, D. barberi: Southern corn rootworm or spotted cucumber beetle; Diabrotica undecimpunctata howardi, and the Mexican corn rootworm, D. virgifera zeae. In one embodiment, the insecticidal activity is against a Diabrotica species.

To determine the percent identity of two amino acid sequences or of two nucleic acid sequences, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. In another embodiment, the comparison is across the entirety of the reference sequence (e.g., across the entirety of SEQ ID NO: 1). The percent identity between two sequences can be determined using techniques similar to those described below, with or without allowing gaps. In calculating percent identity, typically exact matches are counted.

Another non-limiting example of a mathematical algorithm utilized for the comparison of sequences is the algorithm of Needleman and Wunsch, (1970) J. Mol. Biol. 48(3):443-453, used GAP Version 10 software to determine sequence identity or similarity using the following default parameters: % identity and % similarity for a nucleic acid sequence using GAP Weight of 50 and Length Weight of 3, and the nwsgapdna.cmpii scoring matrix; % identity or % similarity for an amino acid sequence using GAP weight of 8 and length weight of 2, and the BLOSUM62 scoring program. Equivalent programs may also be used. “Equivalent program” is used herein to refer to any sequence comparison program that, for any two sequences in question, generates an alignment having identical nucleotide residue matches and an identical percent sequence identity when compared to the corresponding alignment generated by GAP Version 10.

In some embodiments polynucleotides are provided encoding chimeric polypeptides comprising regions of at least two different polypeptides of the disclosure.

The embodiments also encompass nucleic acid molecules encoding variants of the polypeptides of the disclosure. “Variants” of the polypeptide of the disclosure encoding nucleic acid sequences include those sequences encoding the polypeptides of the disclosure but that differ conservatively because of the degeneracy of the genetic code as well as those that are sufficiently identical as discussed above. Naturally occurring allelic variants can be identified using techniques, such as polymerase chain reaction (PCR) and hybridization techniques as outlined below. Variant nucleic acid sequences also include synthetically derived nucleic acid sequences that have been generated, for example, by using site-directed mutagenesis but which still encode the polypeptides of the disclosure as discussed below.

The present disclosure provides isolated or recombinant polynucleotides encoding any of the polypeptides of the disclosure. Those having ordinary skill in the art will readily appreciate that due to the degeneracy of the genetic code, a multitude of nucleotide sequences encoding polypeptides of the disclosure exist.

Changes can be introduced by mutation of the nucleic acid sequences thereby leading to changes in the amino acid sequence of the encoded polypeptides of the disclosure, without altering the biological activity of the proteins. Thus, variant nucleic acid molecules can be created by introducing one or more nucleotide substitutions, additions and/or deletions into the corresponding nucleic acid sequence disclosed herein, such that one or more amino acid substitutions, additions or deletions are introduced into the encoded protein. Mutations can be introduced by standard techniques, such as site-directed mutagenesis and PCR-mediated mutagenesis. Such variant nucleic acid sequences are also encompassed by the present disclosure.

Alternatively, variant nucleic acid sequences can be made by introducing mutations randomly along all or part of the coding sequence, such as by saturation mutagenesis, and the resultant mutants can be screened for ability to confer pesticidal activity to identify mutants that retain activity. Following mutagenesis, the encoded protein can be expressed recombinantly, and the activity of the protein can be determined using standard assay techniques.

The polynucleotides of the disclosure and fragments thereof are optionally used as substrates for a variety of recombination and recursive recombination reactions, in addition to standard cloning methods as set forth in, e.g., Ausubel, Berger and Sambrook, i.e., to produce additional pesticidal polypeptide homologues and fragments thereof with desired properties. Methods for producing a variant of any nucleic acid listed herein comprising recursively recombining such polynucleotide with a second (or more) polynucleotide, thus forming a library of variant polynucleotides are also embodiments of the disclosure, as are the libraries produced, the cells comprising the libraries and any recombinant polynucleotide produced by such methods. Additionally, such methods optionally comprise selecting a variant polynucleotide from such libraries based on pesticidal activity, as is wherein such recursive recombination is done in vitro or in vivo.

A variety of diversity generating protocols, including nucleic acid recursive recombination protocols are available and fully described in the art. The procedures can be used separately, and/or in combination to produce one or more variants of a nucleic acid or set of nucleic acids, as well as variants of encoded proteins. Individually and collectively, these procedures provide robust, widely applicable ways of generating diversified nucleic acids and sets of nucleic acids (including, e.g., nucleic acid libraries) useful, e.g., for the engineering or rapid evolution of nucleic acids, proteins, pathways, cells and/or organisms with new and/or improved characteristics.

While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

The result of any of the diversity generating procedures described herein can be the generation of one or more nucleic acids, which can be selected or screened for nucleic acids with or which confer desirable properties or encoding proteins with or which confer desirable properties. Following diversification by one or more of the methods herein or otherwise available to one of skill, any nucleic acids that are produced can be selected for a desired activity or property, e.g. pesticidal activity or, such activity at a desired pH, etc. This can include identifying any activity that can be detected, for example, in an automated or automatable format, by any of the assays in the art, see, e.g., discussion of screening of insecticidal activity, infra. A variety of related (or even unrelated) properties can be evaluated, in serial or in parallel, at the discretion of the practitioner.

The nucleotide sequences of the embodiments can also be used to isolate corresponding sequences from a bacterial source, including but not limited to a Pseudomonas species. In this manner, methods such as PCR, hybridization, and the like can be used to identify such sequences based on their sequence homology to the sequences set forth herein. Sequences that are selected based on their sequence identity to the entire sequences set forth herein or to fragments thereof are encompassed by the embodiments. Such sequences include sequences that are orthologs of the disclosed sequences. The term “orthologs” refers to genes derived from a common ancestral gene and which are found in different species as a result of speciation. Genes found in different species are considered orthologs when their nucleotide sequences and/or their encoded protein sequences share substantial identity as defined elsewhere herein. Functions of orthologs are often highly conserved among species.

In a PCR approach, oligonucleotide primers can be designed for use in PCR reactions to amplify corresponding DNA sequences from cDNA or genomic DNA extracted from any organism of interest. Methods for designing PCR primers and PCR cloning can be found in Sambrook, et al., (1989) Molecular Cloning: A Laboratory Manual (2d ed., Cold Spring Harbor Laboratory Press, Plainview, New York), hereinafter “Sambrook”. See also, Innis, et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Methods of PCR include, but are not limited to, methods using paired primers, nested primers, single specific primers, degenerate primers, gene-specific primers, vector-specific primers, partially-mismatched primers, and the like.

To identify potential polypeptides of the disclosure from bacterium collections, the bacterial cell lysates can be screened with antibodies generated against a polypeptide of the disclosure using Western blotting and/or ELISA methods. This type of assays can be performed in a high throughput fashion. Positive samples can be further analyzed by various techniques such as antibody based protein purification and identification.

Alternatively, mass spectrometry based protein identification method can be used to identify homologs of polypeptides of the disclosure using protocols in the literatures (Scott Patterson, (1998), 10.22, 1-24, Current Protocol in Molecular Biology published by John Wiley & Son Inc). Specifically, LC-MS/MS based protein identification method is used to associate the MS data of given cell lysate or desired molecular weight enriched samples (excised from SDS-PAGE gel of relevant molecular weight bands to polypeptides of the disclosure) with sequence information of polypeptides of the disclosure. Any match in peptide sequences indicates the potential of having the homologous proteins in the samples. Additional techniques (protein purification and molecular biology) can be used to isolate the protein and identify the sequences of the homologs.

In hybridization methods, all or part of the pesticidal nucleic acid sequence can be used to screen cDNA or genomic libraries. Methods for construction of such cDNA and genomic libraries can be found in Sambrook and Russell, (2001), supra. The so-called hybridization probes may be genomic DNA fragments, cDNA fragments, RNA fragments or other oligonucleotides and may be labeled with a detectable group such as 32P or any other detectable marker, such as other radioisotopes, a fluorescent compound, an enzyme or an enzyme co-factor. Probes for hybridization can be made by labeling synthetic oligonucleotides based on the known polypeptide of the disclosure-encoding nucleic acid sequence disclosed herein. Degenerate primers designed based on conserved nucleotides or amino acid residues in the nucleic acid sequence or encoded amino acid sequence can additionally be used. The probe typically comprises a region of nucleic acid sequence that hybridizes under stringent conditions to at least about 12, at least about 25, at least about 50, 75, 100, 125, 150, 175 or 200 consecutive nucleotides of nucleic acid sequence encoding a polypeptide of the disclosure or a fragment or variant thereof. Methods for the preparation of probes for hybridization can be found in Sambrook and Russell, (2001), supra, herein incorporated by reference.

Hybridization may be carried out under stringent conditions. “Stringent conditions” or “stringent hybridization conditions” is used herein to refer to conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length

### Compositions

Compositions comprising at least one polypeptide of the disclosure are also embraced. In one embodiment, the composition comprises a polypeptide of the disclosure and an agriculturally accepted carrier.

One embodiment of the disclosure relates to a composition comprising a polypeptide of the disclosure and an entomopathogenic fungal strain selected from Metarhizium robertsii and Metarhizium anisopliae. In certain embodiments, the fungal entomopathogen comprises a spore, a microsclerotia or conidia. In some embodiments, a fungal entomopathogen has insecticidal activity.

In one embodiment, the disclosure relates to a composition for increasing resistance to a plant pest, pathogen or insect or for increasing plant health and/or yield comprising a polypeptide of the disclosure and one or more entomopathogenic fungal strains selected from the group consisting of Metarhizium anisopliae 15013-1 (NRRL 67073), Metarhizium robertsii 23013-3 (NRRL 67075), Metarhizium anisopliae 3213-1 (NRRL 67074) or any combinations thereof. In another embodiment, the disclosure relates to a composition comprising a polypeptide of the disclosure, an agriculturally accepted carrier, and a fungal entomopathogen selected from the group consisting of Metarhizium anisopliae 15013-1, Metarhizium robertsii 23013-3, Metarhizium anisopliae 3213-1 or any combinations thereof. In a further embodiment, the fungal entomopathogen comprises a spore, conidia or microsclerotia. In another embodiment, the disclosure relates to a composition comprising a polypeptide of the disclosure and one or more entomopathogenic fungal strains selected from the group consisting of Metarhizium anisopliae 15013-1 (NRRL 67073), Metarhizium robertsii 23013-3 (NRRL 67075), Metarhizium anisopliae 3213-1 (NRRL 67074), mutants of these strains, a metabolite or combination of metabolites produced by a strain disclosed herein that exhibits insecticidal activity towards a plant pest, pathogen or insect or any combinations thereof.

### Antibodies

Antibodies to a polypeptide of the disclosure or to variants or fragments thereof are also encompassed. The antibodies of the disclosure include polyclonal and monoclonal antibodies as well as fragments thereof which retain their ability to bind to a polypeptide of the disclosure found in the insect gut.

A kit for detecting the presence of a polypeptide of the disclosure or detecting the presence of a nucleotide sequence encoding a polypeptide of the disclosure in a sample is provided. In one embodiment, the kit provides antibody-based reagents for detecting the presence of a polypeptide of the disclosure in a tissue sample. In another embodiment, the kit provides labeled nucleic acid probes useful for detecting the presence of one or more polynucleotides encoding a polypeptide of the disclosure. The kit is provided along with appropriate reagents and controls for carrying out a detection method, as well as instructions for use of the kit.

### Receptor Identification and Isolation

Receptors to the polypeptide of the disclosure or to variants or fragments thereof are also encompassed. Methods for identifying receptors can be found in Hofmann, et. al., (1988) Eur. J. Biochem. 173:85-91; Gill, et al., (1995) J. Biol. Chem. 27277-27282), which can be employed to identify and isolate the receptor that recognizes the polypeptide of the disclosure using the brush-border membrane vesicles from susceptible insects. In addition to the radioactive labeling method listed in the cited literatures, a polypeptide of the disclosure can be labeled with fluorescent dye and other common labels such as streptavidin. Brush-border membrane vesicles (BBMV) of susceptible insects such as soybean looper and stink bugs can be prepared according to the protocols listed in the references and separated on SDS-PAGE gel and blotted on suitable membrane. Labeled polypeptide of the disclosure can be incubated with blotted membrane of BBMV and labeled polypeptide of the disclosure can be identified with the labeled reporters.

Identification of protein band(s) that interact with can be detected by N-terminal amino acid gas phase sequencing or mass spectrometry based protein identification method (Patterson, (1998) 10.22, 1-24, Current Protocol in Molecular Biology published by John Wiley & Son Inc). Once the protein is identified, the corresponding gene can be cloned from genomic DNA or cDNA library of the susceptible insects and binding affinity can be measured directly with the polypeptide of the disclosure. Receptor function for insecticidal activity by the polypeptide of the disclosure can be verified by accomplished by RNAi type of gene knock out method (Rajagopal, et al., (2002) J. Biol. Chem. 277:46849-46851).

### Nucleotide Constructs, Expression Cassettes and Vectors

The use of the term “nucleotide constructs” herein is not intended to limit the embodiments to nucleotide constructs comprising DNA. Those of ordinary skill in the art will recognize that nucleotide constructs particularly polynucleotides and oligonucleotides composed of ribonucleotides and combinations of ribonucleotides and deoxyribonucleotides may also be employed in the methods disclosed herein. The nucleotide constructs, nucleic acids, and nucleotide sequences of the embodiments additionally encompass all complementary forms of such constructs, molecules, and sequences. Further, the nucleotide constructs, nucleotide molecules, and nucleotide sequences of the embodiments encompass all nucleotide constructs, molecules, and sequences which can be employed in the methods of the embodiments for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs, nucleic acids, and nucleotide sequences of the embodiments also encompass all forms of nucleotide constructs including, but not limited to, single-stranded forms, double-stranded forms, hairpins, stem-and-loop structures and the like.

A further embodiment relates to a transformed organism such as an organism selected from plant and insect cells, bacteria, yeast, baculovirus, protozoa, nematodes and algae. The transformed organism comprises a DNA molecule of the embodiments, an expression cassette comprising the DNA molecule or a vector comprising the expression cassette, which may be stably incorporated into the genome of the transformed organism.

The sequences of the embodiments are provided in DNA constructs for expression in the organism of interest. The construct will include 5′ and 3′ regulatory sequences operably linked to a sequence of the embodiments. The term “operably linked” as used herein refers to a functional linkage between a promoter and a second sequence, wherein the promoter sequence initiates and mediates transcription of the DNA sequence corresponding to the second sequence. Generally, operably linked means that the nucleic acid sequences being linked are contiguous and where necessary to join two protein coding regions in the same reading frame. The construct may additionally contain at least one additional gene to be cotransformed into the organism. Alternatively, the additional gene(s) can be provided on multiple DNA constructs.

Such a DNA construct is provided with a plurality of restriction sites for insertion of the polypeptide of the disclosure gene sequence of the disclosure to be under the transcriptional regulation of the regulatory regions. The DNA construct may additionally contain selectable marker genes.

The DNA construct will generally include in the 5′ to 3′ direction of transcription: a transcriptional and translational initiation region (i.e., a promoter), a DNA sequence of the embodiments, and a transcriptional and translational termination region (i.e., termination region) functional in the organism serving as a host. The transcriptional initiation region (i.e., the promoter) may be native, analogous, foreign or heterologous to the host organism and/or to the sequence of the embodiments. Additionally, the promoter may be the natural sequence or alternatively a synthetic sequence. The term “foreign” as used herein indicates that the promoter is not found in the native organism into which the promoter is introduced. Where the promoter is “foreign” or “heterologous” to the sequence of the embodiments, it is intended that the promoter is not the native or naturally occurring promoter for the operably linked sequence of the embodiments. As used herein, a chimeric gene comprises a coding sequence operably linked to a transcription initiation region that is heterologous to the coding sequence. Where the promoter is a native or natural sequence, the expression of the operably linked sequence is altered from the wild-type expression, which results in an alteration in phenotype.

In some embodiments, the DNA construct comprises a polynucleotide encoding a polypeptide of the disclosure.

In some embodiments, the DNA construct comprises a polynucleotide encoding a chimeric polypeptide of the disclosure.

In some embodiments, the DNA construct comprises a polynucleotide encoding a fusion protein comprising a polypeptide of the disclosure.

In some embodiments, the DNA construct may also include a transcriptional enhancer sequence. As used herein, the term an “enhancer” refers to a DNA sequence which can stimulate promoter activity, and may be an innate element of the promoter or a heterologous element inserted to enhance the level or tissue-specificity of a promoter. Various enhancers including for example, introns with gene expression enhancing properties in plants (US Patent Application Publication Number 2009/0144863, the ubiquitin intron (i.e., the maize ubiquitin intron 1 (see, for example, NCBI sequence S94464)), the omega enhancer or the omega prime enhancer (Gallie, et al., (1989) Molecular Biology of RNA ed. Cech (Liss, New York) 237-256 and Gallie, et al., (1987) Gene 60:217-25), the CaMV 35S enhancer (see, e.g., Benfey, et al., (1990) EMBO J. 9:1685-96) and the enhancers of U.S. Pat. No. 7,803,992 may also be used, each of which is incorporated by reference. U.S. Pat. No. 8,785,612 discloses the sugarcane bacilliform badnavirus (SCBV) transcriptional enhancer. The above list of transcriptional enhancers is not meant to be limiting. Any appropriate transcriptional enhancer can be used in the embodiments.

The termination region may be native with the transcriptional initiation region, may be native with the operably linked DNA sequence of interest, may be native with the plant host or may be derived from another source (i.e., foreign or heterologous to the promoter, the sequence of interest, the plant host or any combination thereof).

Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903 and Joshi, et al., (1987) Nucleic Acid Res. 15:9627-9639. Other useful transcription terminators for expression of transgenes in plants include the transcription terminators MYB2, KTI1, PIP1, EF1A2, and MTH1 of U.S. Pat. No. 8,741,634.

Where appropriate, a nucleic acid may be optimized for increased expression in the host organism. Thus, where the host organism is a plant, the synthetic nucleic acids can be synthesized using plant-preferred codons for improved expression. See, for example, Campbell and Gowri, (1990) Plant Physiol. 92:1-11 for a discussion of host-preferred usage. For example, although nucleic acid sequences of the embodiments may be expressed in both monocotyledonous and dicotyledonous plant species, sequences can be modified to account for the specific preferences and GC content preferences of monocotyledons or dicotyledons as these preferences have been shown to differ (Murray et al. (1989) Nucleic Acids Res. 17:477-498). Thus, the maize-preferred codon for an amino acid may be derived from known gene sequences from maize. Maize usage for 28 genes from maize plants is listed in Table 4 of Murray, et al., supra. Methods are available in the art for synthesizing plant-preferred genes. See, for example, Murray, et al., (1989) Nucleic Acids Res. 17:477-498, and Liu H et al. Mol Bio Rep 37:677-684, 2010, herein incorporated by reference. A Zea maize usage table can be also found at kazusa.or.jp//cgi-bin/show.cgi?species=4577, which can be accessed using the www prefix.

A Glycine max usage table can be found at kazusa.or.jp//cgi-bin/show.cgi?species=3847&aa=1&style=N, which can be accessed using the www prefix.

In some embodiments, the recombinant nucleic acid molecule encoding a polypeptide of the disclosure has maize optimized codons.

Additional sequence modifications are known to enhance gene expression in a cellular host. These include elimination of sequences encoding spurious polyadenylation signals, exon-intron splice site signals, transposon-like repeats, and other well-characterized sequences that may be deleterious to gene expression. The GC content of the sequence may be adjusted to levels average for a given cellular host, as calculated by reference to known genes expressed in the host cell. The term “host cell” as used herein refers to a cell which contains a vector and supports the replication and/or expression of the expression vector is intended. Host cells may be prokaryotic cells such as E. coli or eukaryotic cells such as yeast, insect, amphibian or mammalian cells or monocotyledonous or dicotyledonous plant cells. An example of a monocotyledonous host cell is a maize host cell. When possible, the sequence is modified to avoid predicted hairpin secondary mRNA structures.

The expression cassettes may additionally contain 5′ leader sequences. Such leader sequences can act to enhance translation. Translation leaders include: picornavirus leaders, for example, EMCV leader (Encephalomyocarditis 5′ noncoding region) (Elroy-Stein, et al., (1989) Proc. Natl. Acad. Sci. USA 86:6126-6130); potyvirus leaders, for example, TEV leader (Tobacco Etch Virus) (Gallie, et al., (1995) Gene 165(2):233-238), MDMV leader (Maize Dwarf Mosaic Virus), human immunoglobulin heavy-chain binding protein (BiP) (Macejak, et al., (1991) Nature 353:90-94); untranslated leader from the coat protein mRNA of alfalfa mosaic virus (AMV RNA 4) (Jobling, et al., (1987) Nature 325:622-625); tobacco mosaic virus leader (TMV) (Gallie, et al., (1989) in Molecular Biology of RNA, ed. Cech (Liss, New York), pp. 237-256) and maize chlorotic mottle virus leader (MCMV) (Lommel, et al., (1991) Virology 81:382-385). See also, Della-Cioppa, et al., (1987) Plant Physiol. 84:965-968. Such constructs may also contain a “signal sequence” or “leader sequence” to facilitate co-translational or post-translational transport of the peptide to certain intracellular structures such as the chloroplast (or other plastid), endoplasmic reticulum or Golgi apparatus.

“Signal sequence” as used herein refers to a sequence that is known or suspected to result in cotranslational or post-translational peptide transport across the cell membrane. In eukaryotes, this typically involves secretion into the Golgi apparatus, with some resulting glycosylation. Insecticidal toxins of bacteria are often synthesized as protoxins, which are proteolytically activated in the gut of the target pest (Chang, (1987) Methods Enzymol. 153:507-516). In some embodiments, the signal sequence is located in the native sequence or may be derived from a sequence of the embodiments. “Leader sequence” as used herein refers to any sequence that when translated, results in an amino acid sequence sufficient to trigger co-translational transport of the peptide chain to a subcellular organelle. Thus, this includes leader sequences targeting transport and/or glycosylation by passage into the endoplasmic reticulum, passage to vacuoles, plastids including chloroplasts, mitochondria, and the like. Nuclear-encoded proteins targeted to the chloroplast thylakoid lumen compartment have a characteristic bipartite transit peptide, composed of a stromal targeting signal peptide and a lumen targeting signal peptide. The stromal targeting information is in the amino-proximal portion of the transit peptide. The lumen targeting signal peptide is in the carboxyl-proximal portion of the transit peptide, and contains all the information for targeting to the lumen. Recent research in proteomics of the higher plant chloroplast has achieved in the identification of numerous nuclear-encoded lumen proteins (Kieselbach et al. FEBS LETT 480:271-276, 2000; Peltier et al. Plant Cell 12:319-341, 2000; Bricker et al. Biochim. Biophys Acta 1503:350-356, 2001), the lumen targeting signal peptide of which can potentially be used in accordance with the present disclosure. About 80 proteins from Arabidopsis, as well as homologous proteins from spinach and garden pea, are reported by Kieselbach et al., Photosynthesis Research, 78:249-264, 2003. In particular, Table 2 of this publication, which is incorporated into the description herewith by reference, discloses 85 proteins from the chloroplast lumen, identified by their accession number (see also US Patent Application Publication 2009/09044298). In addition, the recently published draft version of the rice genome (Goff et al, Science 296:92-100, 2002) is a suitable source for lumen targeting signal peptide which may be used in accordance with the present disclosure.

Suitable chloroplast transit peptides (CTP) include chimeric CT's comprising but not limited to: an N-terminal domain, a central domain or a C-terminal domain from a CTP from Oryza sativa 1-decoy-D xylose-5-Phosphate Synthase Oryza sativa-Superoxide dismutase Oryza sativa-soluble starch synthase Oryza sativa-NADP-dependent Malic acid enzyme Oryza sativa-Phospho-2-dehydro-3-deoxyheptonate Aldolase 2 Oryza sativa-L-Ascorbate peroxidase 5 Oryza sativa-Phosphoglucan water dikinase, Zea mays ssRUBISCO, Zea mays-beta-glucosidase, Zea mays-Malate dehydrogenase, Zea mays Thioredoxin M-type (U.S. Pat. No. 9,150,625); a chloroplast transit peptide of US Patent Application Publication Number US20130210114.

The gene encoding a polypeptide of the disclosure to be targeted to the chloroplast may be optimized for expression in the chloroplast to account for differences in usage between the plant nucleus and this organelle. In this manner, the nucleic acids of interest may be synthesized using chloroplast-preferred sequences.

In preparing the expression cassette, the various DNA fragments may be manipulated to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites or the like. For this purpose, in vitro mutagenesis, primer repair, restriction, annealing, resubstitutions, e.g., transitions and transversions, may be involved.

A number of promoters can be used in the practice of the embodiments. The promoters can be selected based on the desired outcome. The nucleic acids can be combined with constitutive, tissue-preferred, inducible or other promoters for expression in the host organism. Suitable constitutive promoters for use in a plant host cell include, for example, the core promoter of the Rsyn7 promoter and other constitutive promoters disclosed in WO 1999/43838 and U.S. Pat. No. 6,072,050; the core CaMV 35S promoter (Odell, et al., (1985) Nature 313:810-812); rice actin (McElroy, et al., (1990) Plant Cell 2:163-171); ubiquitin (Christensen, et al., (1989) Plant Mol. Biol. 12:619-632 and Christensen, et al., (1992) Plant Mol. Biol. 18:675-689); pEMU (Last, et al., (1991) Theor. Appl. Genet. 81:581-588); MAS (Velten, et al., (1984) EMBO J. 3:2723-2730), U.S. Pat. Nos. 8,168,859, 8,420,797; Ubiquitin transcriptional regulatory elements and transcriptional regulatory expression element group are disclosed in U.S. Pat. No. 9,062,316; ALS promoter (U.S. Pat. No. 5,659,026) and the like. The Soybean ADF1 constitutive promoter is disclosed in US Patent Application Publication US20150184174. The Soybean CCP1 constitutive promoter is disclosed in US Patent Application Publication US20150167011. Other constitutive promoters include, for example, those discussed in U.S. Pat. Nos. 5,608,149; 5,608,144; 5,604,121; 5,569,597; 5,466,785; 5,399,680; 5,268,463; 5,608,142 and 6,177,611. Transcriptional initiation regions isolated from a blueberry red ringspot virus (BRRV) are disclosed in U.S. Pat. No. 8,895,716. Transcriptional initiation regions isolated from a cacao swollen shoot virus (CSSV) are disclosed in U.S. Pat. No. 8,962,916.

Depending on the desired outcome, it may be beneficial to express the gene from an inducible promoter. Of particular interest for regulating the expression of the nucleotide sequences of the embodiments in plants are wound-inducible promoters. Such wound-inducible promoters, may respond to damage caused by insect feeding, and include potato proteinase inhibitor (pin II) gene (Ryan, (1990) Ann. Rev. Phytopath. 28:425-449; Duan, et al., (1996) Nature Biotechnology 14:494-498); wun1 and wun2, U.S. Pat. No. 5,428,148; win1 and win2 (Stanford, et al., (1989) Mol. Gen. Genet. 215:200-208); systemin (McGurl, et al., (1992) Science 225:1570-1573); WIP1 (Rohmeier, et al., (1993) Plant Mol. Biol. 22:783-792; Eckelkamp, et al., (1993) FEBS Letters 323:73-76); MPI gene (Corderok, et al., (1994) Plant J. 6(2):141-150) and the like, herein incorporated by reference.

Additionally, pathogen-inducible promoters may be employed in the methods and nucleotide constructs of the embodiments. Such pathogen-inducible promoters include those from pathogenesis-related proteins (PR proteins), which are induced following infection by a pathogen; e.g., PR proteins, SAR proteins, beta-1,3-glucanase, chitinase, etc. See, for example, Redolfi, et al., (1983) Neth. J. Plant Pathol. 89:245-254; Uknes, et al., (1992) Plant Cell 4: 645-656 and Van Loon, (1985) Plant Mol. Virol. 4:111-116. See also, WO 1999/43819, herein incorporated by reference.

Of interest are promoters that are expressed locally at or near the site of pathogen infection. See, for example, Marineau, et al., (1987) Plant Mol. Biol. 9:335-342; Matton, et al., (1989) Molecular Plant-Microbe Interactions 2:325-331; Somsisch, et al., (1986) Proc. Natl. Acad. Sci. USA 83:2427-2430; Somsisch, et al., (1988) Mol. Gen. Genet. 2:93-98 and Yang, (1996) Proc. Natl. Acad. Sci. USA 93:14972-14977. See also, Chen, et al., (1996) Plant J. 10:955-966; Zhang, et al., (1994) Proc. Natl. Acad. Sci. USA 91:2507-2511; Warner, et al., (1993) Plant J. 3:191-201; Siebertz, et al., (1989) Plant Cell 1:961-968; U.S. Pat. No. 5,750,386 (nematode-inducible) and the references cited therein. Of particular interest is the inducible promoter for the maize PRms gene, whose expression is induced by the pathogen Fusarium moniliforme (see, for example, Cordero, et al., (1992) Physiol. Mol. Plant Path. 41:189-200).

Chemical-regulated promoters can be used to modulate the expression of a gene in a plant through the application of an exogenous chemical regulator. Depending upon the objective, the promoter may be a chemical-inducible promoter, where application of the chemical induces gene expression or a chemical-repressible promoter, where application of the chemical represses gene expression. Chemical-inducible promoters include the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena, et al., (1991) Proc. Natl. Acad. Sci. USA 88:10421-10425 and McNellis, et al., (1998) Plant J. 14(2):247-257) and tetracycline-inducible and tetracycline-repressible promoters (see, for example, Gatz, et al., (1991) Mol. Gen. Genet. 227:229-237 and U.S. Pat. Nos. 5,814,618 and 5,789,156), herein incorporated by reference.

Tissue-preferred promoters can be utilized to target enhanced a polypeptide of the disclosure expression within a particular plant tissue. Tissue-preferred promoters include those discussed in Yamamoto, et al., (1997) Plant J. 12(2)255-265; Kawamata, et al., (1997) Plant Cell Physiol. 38(7):792-803; Hansen, et al., (1997) Mol. Gen Genet. 254(3):337-343; Russell, et al., (1997) Transgenic Res. 6(2):157-168; Rinehart, et al., (1996) Plant Physiol. 112(3):1331-1341; Van Camp, et al., (1996) Plant Physiol. 112(2):525-535; Canevascini, et al., (1996) Plant Physiol. 112(2):513-524; Yamamoto, et al., (1994) Plant Cell Physiol. 35(5):773-778; Lam, (1994) Results Probl. Cell Differ. 20:181-196; Orozco, et al., (1993) Plant Mol Biol. 23(6):1129-1138; Matsuoka, et al., (1993) Proc Natl. Acad. Sci. USA 90(20):9586-9590 and Guevara-Garcia, et al., (1993) Plant J. 4(3):495-505. Additional tissue specific promoters include the promoters of U.S. Pat. Nos. 8,816,152 and 9,150,624. Such promoters can be modified, if necessary, for weak expression.

Leaf-preferred promoters can be found in Yamamoto, et al., (1997) Plant J. 12(2):255-265; Kwon, et al., (1994) Plant Physiol. 105:357-67; Yamamoto, et al., (1994) Plant Cell Physiol. 35(5):773-778; Gotor, et al., (1993) Plant J. 3:509-18; Orozco, et al., (1993) Plant Mol. Biol. 23(6):1129-1138 and Matsuoka, et al., (1993) Proc. Natl. Acad. Sci. USA 90(20):9586-9590.

US Patent Application-preferred or root-specific promoters can be found in Hire, et al., (1992) Plant Mol. Biol. 20(2):207-218 (soybean root-specific glutamine synthetase gene); Keller and Baumgartner, (1991) Plant Cell 3(10):1051-1061 (root-specific control element in the GRP 1.8 gene of French bean); Sanger, et al., (1990) Plant Mol. Biol. 14(3):433-443 (root-specific promoter of the mannopine synthase (MAS) gene of Agrobacterium tumefaciens) and Miao, et al., (1991) Plant Cell 3(1):11-22 (full-length cDNA clone encoding cytosolic glutamine synthetase (GS), which is expressed in roots and root nodules of soybean). See also, Bogusz, et al., (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi, (1991) describe their analysis of the promoters of the highly expressed roIC and roID root-inducing genes of Agrobacterium rhizogenes (see, Plant Science (Limerick) 79(1):69-76). They concluded that enhancer and tissue-preferred DNA determinants are dissociated in those promoters. Teeri, et al., (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see, EMBO J. 8(2):343-350). The TR1′ gene fused to nptII (neomycin phosphotransferase II) showed similar characteristics. Additional root-preferred promoters include the VfENOD-GRP3 gene promoter (Kuster, et al., (1995) Plant Mol. Biol. 29(4):759-772) and roIB promoter (Capana, et al., (1994) Plant Mol. Biol. 25(4):681-691. See also, U.S. Pat. Nos. 5,837,876; 5,750,386; 5,633,363; 5,459,252; 5,401,836; 5,110,732 and 5,023,179. Arabidopsis thaliana root-preferred regulatory sequences are disclosed in US20130117883. US Patent Application Publication Number US20160097054 discloses the sorghum root-preferred promoter PLTP. US Patent Application Publication Number US20160145634 discloses the sorghum root-preferred promoter TIP2-3. U.S. Pat. No. 8,916,377 discloses the sorghum root-preferred promoter RCc3.

“Seed-preferred” promoters include both “seed-specific” promoters (those promoters active during seed development such as promoters of seed storage proteins) as well as “seed-germinating” promoters (those promoters active during seed germination). See, Thompson, et al., (1989) BioEssays 10:108, herein incorporated by reference. Such seed-preferred promoters include, but are not limited to, Cim1 (cytokinin-induced message); cZ19B1 (maize 19 kDa zein); and milps (myo-inositol-1-phosphate synthase) (see, U.S. Pat. No. 6,225,529, herein incorporated by reference). Gamma-zein and Glb-1 are endosperm-specific promoters. For dicots, seed-specific promoters include, but are not limited to, Kunitz trypsin inhibitor 3 (KTi3) (Jofuku and Goldberg, (1989) Plant Cell 1:1079-1093), bean β-phaseolin, napin, β-conglycinin, glycinin 1, soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, globulin 1, etc. See also, WO 2000/12733, where seed-preferred promoters from end1 and end2 genes are disclosed; herein incorporated by reference. In dicots, seed specific promoters include but are not limited to seed coat promoter from Arabidopsis, pBAN; and the early seed promoters from Arabidopsis, p26, p63, and p63tr (U.S. Pat. Nos. 7,294,760 and 7,847,153). A promoter that has “preferred” expression in a particular tissue is expressed in that tissue to a greater degree than in at least one other plant tissue. Some tissue-preferred promoters show expression almost exclusively in the particular tissue.

Where low level expression is desired, weak promoters will be used. Generally, the term “weak promoter” as used herein refers to a promoter that drives expression of a coding sequence at a low level. By low level expression at levels of between about 1/1000 transcripts to about 1/100,000 transcripts to about 1/500,000 transcripts is intended. Alternatively, it is recognized that the term “weak promoters” also encompasses promoters that drive expression in only a few cells and not in others to give a total low level of expression. Where a promoter drives expression at unacceptably high levels, portions of the promoter sequence can be deleted or modified to decrease expression levels.

Such weak constitutive promoters include, for example the core promoter of the Rsyn7 promoter (WO 1999/43838 and U.S. Pat. No. 6,072,050), the core 35S CaMV promoter, and the like. Other constitutive promoters include, for example, those disclosed in U.S. Pat. Nos. 5,608,149; 5,608,144; 5,604,121; 5,569,597; 5,466,785; 5,399,680; 5,268,463; 5,608,142, 6,177,611, and 8,697,857, herein incorporated by reference.

Chimeric or hybrid promoters include those disclosed in U.S. Pat. Nos. 8,846,892, 8,822,666, and 9,181,560.

The above list of promoters is not meant to be limiting. Any appropriate promoter can be used in the embodiments.

Generally, the expression cassette will comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones and 2,4-dichlorophenoxyacetate (2,4-D). Additional examples of suitable selectable marker genes include, but are not limited to, genes encoding resistance to chloramphenicol (Herrera Estrella, et al., (1983) EMBO J. 2:987-992); methotrexate (Herrera Estrella, et al., (1983) Nature 303:209-213 and Meijer, et al., (1991) Plant Mol. Biol. 16:807-820); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-136); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J. 6:2513-2518). See generally, Yarranton, (1992) Curr. Opin. Biotech. 3:506-511; Christopherson, et al., (1992) Proc. Natl. Acad. Sci. USA 89:6314-6318; Yao, et al., (1992) Cell 71:63-72; Reznikoff, (1992) Mol. Microbiol. 6:2419-2422; Barkley, et al., (1980) in The Operon, pp. 177-220; Hu, et al., (1987) Cell 48:555-566; Brown, et al., (1987) Cell 49:603-612; Figge, et al., (1988) Cell 52:713-722; Deuschle, et al., (1989) Proc. Natl. Acad. Sci. USA 86:5400-5404; Fuerst, et al., (1989) Proc. Natl. Acad. Sci. USA 86:2549-2553; Deuschle, et al., (1990) Science 248:480-483; Gossen, (1993) Ph.D. Thesis, University of Heidelberg; Reines, et al., (1993) Proc. Natl. Acad. Sci. USA 90:1917-1921; Labow, et al., (1990) Mol. Cell. Biol. 10:3343-3356; Zambretti, et al., (1992) Proc. Natl. Acad. Sci. USA 89:3952-3956; Baim, et al., (1991) Proc. Natl. Acad. Sci. USA 88:5072-5076; Wyborski, et al., (1991) Nucleic Acids Res. 19:4647-4653; Hillenand-Wissman, (1989) Topics Mol. Struc. Biol. 10:143-162; Degenkolb, et al., (1991) Antimicrob. Agents Chemother. 35:1591-1595; Kleinschnidt, et al., (1988) Biochemistry 27:1094-1104; Bonin, (1993) Ph.D. Thesis, University of Heidelberg; Gossen, et al., (1992) Proc. Natl. Acad. Sci. USA 89:5547-5551; Oliva, et al., (1992) Antimicrob. Agents Chemother. 36:913-919; Hlavka, et al., (1985) Handbook of Experimental Pharmacology, Vol. 78 (Springer-Verlag, Berlin) and Gill, et al., (1988) Nature 334:721-724. Such disclosures are herein incorporated by reference.

The above list of selectable marker genes is not meant to be limiting. Any selectable marker gene can be used in the embodiments.

### Plant Transformation

The methods of the embodiments involve introducing a polypeptide or polynucleotide into a plant. “Introducing” is as used herein means presenting to the plant the polynucleotide or polypeptide in such a manner that the sequence gains access to the interior of a cell of the plant. The methods of the embodiments do not depend on a particular method for introducing a polynucleotide or polypeptide into a plant, only that the polynucleotide or polypeptides gains access to the interior of at least one cell of the plant. Methods for introducing polynucleotide or polypeptides into plants include stable transformation methods, transient transformation methods, and virus-mediated methods.

“Stable transformation” is as used herein means that the nucleotide construct introduced into a plant integrates into the genome of the plant and is capable of being inherited by the progeny thereof. “Transient transformation” as used herein means that a polynucleotide is introduced into the plant and does not integrate into the genome of the plant or a polypeptide is introduced into a plant. “Plant” as used herein refers to whole plants, plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells and pollen).

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway, et al., (1986) Biotechniques 4:320-334), electroporation (Riggs, et al., (1986) Proc. Natl. Acad. Sci. USA 83:5602-5606), Agrobacterium-mediated transformation (U.S. Pat. Nos. 5,563,055 and 5,981,840), direct gene transfer (Paszkowski, et al., (1984) EMBO J. 3:2717-2722) and ballistic particle acceleration (see, for example, U.S. Pat. Nos. 4,945,050; 5,879,918; 5,886,244 and 5,932,782; Tomes, et al., (1995) in Plant Cell, Tissue, and Organ Culture: Fundamental Methods, ed. Gamborg and Phillips, (Springer-Verlag, Berlin) and McCabe, et al., (1988) Biotechnology 6:923-926) and Lecl transformation (WO 00/28058). For potato transformation see, Tu, et al., (1998) Plant Molecular Biology 37:829-838 and Chong, et al., (2000) Transgenic Research 9:71-78. Additional transformation procedures can be found in Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783 and 5,324,646; Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, New York), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotechnology 14:745-750 (maize via Agrobacterium tumefaciens); all of which are herein incorporated by reference.

In specific embodiments, the sequences of the embodiments can be provided to a plant using a variety of transient transformation methods. Such transient transformation methods include, but are not limited to, the introduction of the polynucleotide or variants and fragments thereof directly into the plant or the introduction of the polypeptide of the disclosure transcript into the plant. Such methods include, for example, microinjection or particle bombardment. See, for example, Crossway, et al., (1986) Mol Gen. Genet. 202:179-185; Nomura, et al., (1986) Plant Sci. 44:53-58; Hepler, et al., (1994) Proc. Natl. Acad. Sci. 91:2176-2180 and Hush, et al., (1994) The Journal of Cell Science 107:775-784, all of which are herein incorporated by reference. Alternatively, the polynucleotide can be transiently transformed into the plant using techniques such as a viral vector system and the precipitation of the polynucleotide in a manner that precludes subsequent release of the DNA. Thus, transcription from the particle-bound DNA can occur, but the frequency with which it is released to become integrated into the genome is greatly reduced. Such methods include the use of particles coated with polyethylimine (PEI; Sigma #P3143).

Methods for the targeted insertion of a polynucleotide at a specific location in the plant genome include the insertion of the polynucleotide at a desired genomic location is achieved using a site-specific recombination system. See, for example, WO 1999/25821, WO 1999/25854, WO 1999/25840, WO 1999/25855 and WO 1999/25853, all of which are herein incorporated by reference. Briefly, the polynucleotide of the embodiments can be contained in transfer cassette flanked by two non-identical recombination sites. The transfer cassette is introduced into a plant have stably incorporated into its genome a target site which is flanked by two non-identical recombination sites that correspond to the sites of the transfer cassette. An appropriate recombinase is provided and the transfer cassette is integrated at the target site. The polynucleotide of interest is thereby integrated at a specific chromosomal position in the plant genome.

Plant transformation vectors may be comprised of one or more DNA vectors needed for achieving plant transformation. For example, it is a common practice in the art to utilize plant transformation vectors that are comprised of more than one contiguous DNA segment. These vectors are often referred to in the art as “binary vectors”. Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a “gene of interest” (a gene engineered to be capable of expression in a plant cell for which generation of transgenic plants is desired). Also, present on this plasmid vector are sequences required for bacterial replication. The cis-acting sequences are arranged in a fashion to allow efficient transfer into plant cells and expression therein. For example, the selectable marker gene and the pesticidal gene are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as is understood in the art (Hellens and Mullineaux, (2000) Trends in Plant Science 5:446-451). Several types of Agrobacterium strains (e.g. LBA4404, GV3101, EHA101, EHA105, etc.) can be used for plant transformation. The second plasmid vector is not necessary for transforming the plants by other methods such as microprojection, microinjection, electroporation, polyethylene glycol, etc.

In general, plant transformation methods involve transferring heterologous DNA into target plant cells (e.g., immature or mature embryos, suspension cultures, undifferentiated callus, protoplasts, etc.), followed by applying a maximum threshold level of appropriate selection (depending on the selectable marker gene) to recover the transformed plant cells from a group of untransformed cell mass. Following integration of heterologous foreign DNA into plant cells, one then applies a maximum threshold level of appropriate selection in the medium to kill the untransformed cells and separate and proliferate the putatively transformed cells that survive from this selection treatment by transferring regularly to a fresh medium. By continuous passage and challenge with appropriate selection, one identifies and proliferates the cells that are transformed with the plasmid vector. Molecular and biochemical methods can then be used to confirm the presence of the integrated heterologous gene of interest into the genome of the transgenic plant.

Explants are typically transferred to a fresh supply of the same medium and cultured routinely. Subsequently, the transformed cells are differentiated into shoots after placing on regeneration medium supplemented with a maximum threshold level of selecting agent. The shoots are then transferred to a selective rooting medium for recovering rooted shoot or plantlet. The transgenic plantlet then grows into a mature plant and produces fertile seeds (e.g., Hiei, et al., (1994) The Plant Journal 6:271-282; Ishida, et al., (1996) Nature Biotechnology 14:745-750). Explants are typically transferred to a fresh supply of the same medium and cultured routinely. A general description of the techniques and methods for generating transgenic plants are found in Ayres and Park, (1994) Critical Reviews in Plant Science 13:219-239 and Bommineni and Jauhar, (1997) Maydica 42:107-120. Since the transformed material contains many cells; both transformed and non-transformed cells are present in any piece of subjected target callus or tissue or group of cells. The ability to kill non-transformed cells and allow transformed cells to proliferate results in transformed plant cultures. Often, the ability to remove non-transformed cells is a limitation to rapid recovery of transformed plant cells and successful generation of transgenic plants.

The cells that have been transformed may be grown into plants in accordance with conventional ways. See, for example, McCormick, et al., (1986) Plant Cell Reports 5:81-84. These plants may then be grown, and either pollinated with the same transformed strain or different strains, and the resulting hybrid having constitutive or inducible expression of the desired phenotypic characteristic identified. Two or more generations may be grown to ensure that expression of the desired phenotypic characteristic is stably maintained and inherited and then seeds harvested to ensure that expression of the desired phenotypic characteristic has been achieved.

The nucleotide sequences of the embodiments may be provided to the plant by contacting the plant with a virus or viral nucleic acids. Generally, such methods involve incorporating the nucleotide construct of interest within a viral DNA or RNA molecule. It is recognized that the recombinant proteins of the embodiments may be initially synthesized as part of a viral polyprotein, which later may be processed by proteolysis in vivo or in vitro to produce the desired polypeptide of the disclosure. It is also recognized that such a viral polyprotein, comprising at least a portion of the amino acid sequence of a polypeptide of the embodiments, may have the desired pesticidal activity. Such viral polyproteins and the nucleotide sequences encoding for them are encompassed by the embodiments. Methods for providing plants with nucleotide constructs and producing the encoded proteins in the plants, which involve viral DNA or RNA molecules, for example, U.S. Pat. Nos. 5,889,191; 5,889,190; 5,866,785; 5,589,367 and 5,316,931; herein incorporated by reference.

Methods for transformation of chloroplasts can be used, for example, Svab, et al., (1990) Proc. Natl. Acad. Sci. USA 87:8526-8530; Svab and Maliga, (1993) Proc. Natl. Acad. Sci. USA 90:913-917; Svab and Maliga, (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride, et al., (1994) Proc. Natl. Acad. Sci. USA 91:7301-7305.

The embodiments further relate to plant-propagating material of a transformed plant of the embodiments including, but not limited to, seeds, tubers, corms, bulbs, leaves and cuttings of roots and shoots.

The embodiments may be used for transformation of any plant species, including, but not limited to, monocots and dicots. Examples of plants of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the embodiments include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Plants of the embodiments include crop plants (for example, corn, alfalfa, sunflower, Brassica, soybean, cotton, safflower, peanut, sorghum, wheat, millet, tobacco, etc.), such as corn and soybean plants.

Turf grasses include, but are not limited to: annual bluegrass (Poa annus); annual ryegrass (Lolium multiflorum); Canada bluegrass (Poa compressa); Chewing's fescue (Festuca rubra); colonial bentgrass (Agrostis tenuis); creeping bentgrass (Agrostis palustris); crested wheatgrass (Agropyron desertorum); fairway wheatgrass (Agropyron cristatum); hard fescue (Festuca longifolia); Kentucky bluegrass (Poa pratensis); orchardgrass (Dactylis glomerata); perennial ryegrass (Lolium perenne); red fescue (Festuca rubra); redtop (Agrostis alba); rough bluegrass (Poa trivialis); sheep fescue (Festuca ovina); smooth bromegrass (Bromus inermis); tall fescue (Festuca arundinacea); timothy (Phleum pratense); velvet bentgrass (Agrostis canina); weeping alkaligrass (Puccinellia distans); western wheatgrass (Agropyron smithii); Bermuda grass (Cynodon spp.); St. Augustine grass (Stenotaphrum secundatum); zoysia grass (Zoysia spp.); Bahia grass (Paspalum notatum); carpet grass (Axonopus affinis); centipede grass (Eremochloa ophiuroides); kikuyu grass (Pennisetum clandesinum); seashore paspalum (Paspalum vaginatum); blue gramma (Bouteloua gracilis); buffalo grass (Buchloe dactyloids); sideoats gramma (Bouteloua curtipendula).

Plants of interest include grain plants that provide seeds of interest, oil-seed plants, and leguminous plants. Seeds of interest include grain seeds, such as corn, wheat, barley, rice, sorghum, rye, millet, etc. Oil-seed plants include cotton, soybean, safflower, sunflower, Brassica, maize, alfalfa, palm, coconut, flax, castor, olive, etc. Leguminous plants include beans and peas. Beans include guar, locust bean, fenugreek, soybean, garden beans, cowpea, mung bean, lima bean, fava bean, lentils, chickpea, etc.

### Evaluation of Plant Transformation

Following introduction of heterologous foreign DNA into plant cells, the transformation or integration of heterologous gene in the plant genome is confirmed by various methods such as analysis of nucleic acids, proteins and metabolites associated with the integrated gene.

PCR analysis is a rapid method to screen transformed cells, tissue or shoots for the presence of incorporated gene at the earlier stage before transplanting into the soil (Sambrook and Russell, (2001) Molecular Cloning: A Laboratory Manual. Cold Spring Harbor Laboratory Press, Cold Spring Harbor, N.Y.). PCR is carried out using oligonucleotide primers specific to the gene of interest or Agrobacterium vector background, etc.

Plant transformation may be confirmed by Southern blot analysis of genomic DNA (Sambrook and Russell, (2001) supra). In general, total DNA is extracted from the transformant, digested with appropriate restriction enzymes, fractionated in an agarose gel and transferred to a nitrocellulose or nylon membrane. The membrane or “blot” is then probed with, for example, radiolabeled 32P target DNA fragment to confirm the integration of introduced gene into the plant genome according to standard techniques (Sambrook and Russell, (2001) supra).

In Northern blot analysis, RNA is isolated from specific tissues of transformant, fractionated in a formaldehyde agarose gel, and blotted onto a nylon filter according to standard procedures that are routinely used in the art (Sambrook and Russell, (2001) supra). Expression of RNA encoded by the pesticidal gene is then tested by hybridizing the filter to a radioactive probe derived from a pesticidal gene.

Western blot, biochemical assays and the like may be carried out on the transgenic plants to confirm the presence of protein encoded by the pesticidal gene by standard procedures (Sambrook and Russell, 2001, supra) using antibodies that bind to one or more epitopes present on the polypeptide of the disclosure.

Methods to Introduce Genome Editing Technologies into Plants

In some embodiments, the disclosed polynucleotide compositions can be introduced into the genome of a plant using genome editing technologies or previously introduced polynucleotides in the genome of a plant may be edited using genome editing technologies. For example, the disclosed polynucleotides can be introduced into a desired location in the genome of a plant using double-stranded break technologies such as TALENs, meganucleases, zinc finger nucleases, CRISPR-Cas, and the like. For example, the disclosed polynucleotides can be introduced into a desired location in a genome using a CRISPR-Cas system, for site-specific insertion. The desired location in a plant genome can be any desired target site for insertion, such as a genomic region amenable for breeding or may be a target site located in a genomic window with an existing trait of interest. Existing traits of interest could be either an endogenous trait or a previously introduced trait.

In some embodiments, where the disclosed polynucleotide has previously been introduced into a genome, genome editing technologies may be used to alter or modify the introduced polynucleotide sequence. Site specific modifications that can be introduced into the disclosed polynucleotide compositions include those produced using any method for introducing site specific modification, including, but not limited to, using gene repair oligonucleotides (e.g. US Publication 2013/0019349) or using double-stranded break technologies such as TALENs, meganucleases, zinc finger nucleases, CRISPR-Cas, and the like. Such technologies can be used to modify the previously introduced polynucleotide through the insertion, deletion or substitution of nucleotides within the introduced polynucleotide. Alternatively, double-stranded break technologies can be used to add additional nucleotide sequences to the introduced polynucleotide. Additional sequences that may be added include, additional expression elements, such as enhancer and promoter sequences. In another embodiment, genome editing technologies may be used to position additional insecticidally-active proteins in close proximity to the disclosed polynucleotide compositions disclosed herein within the genome of a plant, to generate molecular stacks of insecticidally-active proteins.

An “altered target site,” “altered target sequence.” “modified target site,” and “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide or (iv) any combination of (i)-(iii).

### Stacking of Traits in Transgenic Plant

Transgenic plants may comprise a stack of one or more insecticidal polynucleotides disclosed herein with one or more additional polynucleotides resulting in the production or suppression of multiple polypeptide sequences. Transgenic plants comprising stacks of polynucleotide sequences can be obtained by either or both of traditional breeding methods or through genetic engineering methods. These methods include, but are not limited to, breeding individual lines each comprising a polynucleotide of interest, transforming a transgenic plant comprising a gene disclosed herein with a subsequent gene and co-transformation of genes into a single plant cell. As used herein, the term “stacked” includes having the multiple traits present in the same plant (i.e., both traits are incorporated into the nuclear genome, one trait is incorporated into the nuclear genome and one trait is incorporated into the genome of a plastid or both traits are incorporated into the genome of a plastid). In one non-limiting example, “stacked traits” comprise a molecular stack where the sequences are physically adjacent to each other. A trait, as used herein, refers to the phenotype derived from a sequence or groups of sequences. Co-transformation of genes can be carried out using single transformation vectors comprising multiple genes or genes carried separately on multiple vectors. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system.

In some embodiments polynucleotides encoding the polypeptide of the disclosure, alone or stacked with one or more additional insect resistance traits can be stacked with one or more additional input traits (e.g., herbicide resistance, fungal resistance, virus resistance, stress tolerance, disease resistance, male sterility, stalk strength, and the like) or output traits (e.g., increased yield, modified starches, improved oil profile, balanced amino acids, high lysine or methionine, increased digestibility, improved fiber quality, drought resistance, and the like). Thus, the polynucleotide embodiments can be used to provide a complete agronomic package of improved crop quality with the ability to flexibly and cost effectively control any number of agronomic pests.

Transgenes useful for stacking include but are not limited to transgenes that confer resistance to insects or disease or herbicides.

Plant disease resistance genes. Plant defenses are often activated by specific interaction between the product of a disease resistance gene (R) in the plant and the product of a corresponding avirulence (Avr) gene in the pathogen. A plant variety can be transformed with cloned resistance gene to engineer plants that are resistant to specific pathogen strains. See, for example, Jones, et al., (1994) Science 266:789 (cloning of the tomato Cf-9 gene for resistance to Cladosporium fulvum); Martin, et al., (1993) Science 262:1432 (tomato Pto gene for resistance to Pseudomonas syringae pv. tomato encodes a protein kinase); Mindrinos, et al., (1994) Cell 78:1089 (Arabidopsis RSP2 gene for resistance to Pseudomonas syringae), McDowell and Woffenden, (2003) Trends Biotechnol. 21(4):178-83 and Toyoda, et al., (2002) Transgenic Res. 11(6):567-82. A plant resistant to a disease is one that is more resistant to a pathogen as compared to the wild type plant.

Genes encoding a Bacillus thuringiensis protein, a derivative thereof or a synthetic polypeptide modeled thereon. See, for example, Geiser, et al., (1986) Gene 48:109, who disclose the cloning and nucleotide sequence of a Bt delta-endotoxin gene. Moreover, DNA molecules encoding delta-endotoxin genes can be purchased from American Type Culture Collection (Rockville, Md.), for example, under ATCC® Accession Numbers 40098, 67136, 31995 and 31998. Other non-limiting examples of Bacillus thuringiensis transgenes being genetically engineered are given in the following patents and patent applications and hereby are incorporated by reference for this purpose: U.S. Pat. Nos. 5,188,960; 5,689,052; 5,880,275; 5,986,177; 6,023,013, 6,060,594, 6,063,597, 6,077,824, 6,620,988, 6,642,030, 6,713,259, 6,893,826, 7,105,332; 7,179,965, 7,208,474; 7,227,056, 7,288,643, 7,323,556, 7,329,736, 7,449,552, 7,468,278, 7,510,878, 7,521,235, 7,544,862, 7,605,304, 7,696,412, 7,629,504, 7,705,216, 7,772,465, 7,790,846, 7,858,849, 9,546,378; US Patent Publication US20160376607 and WO 1991/14778; WO 1999/31248; WO 2001/12731; WO 1999/24581 and WO 1997/40162.

Genes encoding pesticidal proteins may also be stacked including but are not limited to a “pesticidal toxin” or “pesticidal protein” or “insecticidal protein”, which are used herein to refer to a toxin that has toxic activity against one or more insect pests, including, but not limited to, members of the Lepidoptera, Diptera, Hemiptera and Coleoptera orders or the Nematoda phylum or a protein that has homology to such a protein. Pesticidal proteins have been isolated from organisms including, for example, Bacillus sp., Pseudomonas sp., Photorhabdus sp., Xenorhabdus sp., Clostridium bifermentans and Paenibacillus popilliae. Pesticidal proteins include but are not limited to: insecticidal proteins from Pseudomonas sp. such as PSEEN3174 (Monalysin; (2011) PLoS Pathogens 7:1-13); from Pseudomonas protegens strain CHAO and Pf-5 (previously fluorescens) (Pechy-Tarr, (2008) Environmental Microbiology 10:2368-2386; GenBank Accession No. EU400157); from Pseudomonas taiwanensis (Liu, et al., (2010) J. Agric. Food Chem., 58:12343-12349) and from Pseudomonas pseudoalcaligenes (Zhang, et al., (2009) Annals of Microbiology 59:45-50 and Li, et al., (2007) Plant Cell Tiss. Organ Cult. 89:159-168); insecticidal proteins from Photorhabdus sp. and Xenorhabdus sp. (Hinchliffe, et al., (2010) The Open Toxicology Journal, 3:101-118 and Morgan, et al., (2001) Applied and Envir. Micro. 67:2062-2069); U.S. Pat. Nos. 6,048,838, and 6,379,946; a PIP-1 polypeptide of U.S. Pat. No. 9,688,730; an AfIP-1A and/or AfIP-1B polypeptide of U.S. Pat. No. 9,475,847; a PIP-47 polypeptide of US Publication Number US20160186204; an IPD045 polypeptide, an IPD064 polypeptide, an IPD074 polypeptide, an IPD075 polypeptide, and an IPD077 polypeptide of PCT Publication Number WO 2016/114973; an IPD080 polypeptide of PCT Serial Number PCT/US17/56517; an IPD078 polypeptide, an IPD084 polypeptide, an IPD085 polypeptide, an IPD086 polypeptide, an IPD087 polypeptide, an IPD088 polypeptide, and an IPD089 polypeptide of Serial Number PCT/US17/54160; PIP-72 polypeptide of US Patent Publication Number US20160366891; a PtIP-50 polypeptide and a PtIP-65 polypeptide of US Publication Number US20170166921; an IPD098 polypeptide, an IPD059 polypeptide, an IPD108 polypeptide, an IPD109 polypeptide of U.S. Ser. No. 62/521084; a PtIP-83 polypeptide of US Publication Number US20160347799; a PtIP-96 polypeptide of US Publication Number US20170233440; an IPD079 polypeptide of PCT Publication Number WO2017/23486; an IPD082 polypeptide of PCT Publication Number WO 2017/105987, an IPD090 polypeptide of Serial Number PCT/US17/30602, an IPD093 polypeptide of U.S. Ser. No. 62/434020; an IPD103 polypeptide of Serial Number PCT/US17/39376; an IPD101 polypeptide of U.S. Ser. No. 62/438179; an IPD110 polypeptide of U.S. Ser. No. 62/642,644; an IPD113 polypeptide of U.S. Ser. No. 62/642,642; an IPD121 polypeptide of U.S. Ser. No. 62/508,514; and δ-endotoxins including, but not limited to a Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry28, Cry29, Cry30, Cry31, Cry32, Cry33, Cry34, Cry35,Cry36, Cry37, Cry38, Cry39, Cry40, Cry41, Cry42, Cry43, Cry44, Cry45, Cry46, Cry47, Cry49, Cry50, Cry51, Cry52, Cry53, Cry54, Cry55, Cry56, Cry57, Cry58, Cry59, Cry60, Cry61, Cry62, Cry63, Cry64, Cry65, Cry66, Cry67, Cry68, Cry69, Cry70, Cry71, and Cry 72 classes of δ-endotoxin polypeptides and the B. thuringiensis cytolytic cyt1 and cyt2 genes. Members of these classes of B. thuringiensis insecticidal proteins well known to one skilled in the art (see, Crickmore, et al., “Bacillus thuringiensis toxin nomenclature” (2011), at lifesci.sussex.ac.uk/home/Neil_Crickmore/Bt/ which can be accessed on the world-wide web using the “www” prefix).

Examples of δ-endotoxins also include but are not limited to CrylA proteins of U.S. Pat. Nos. 5,880,275, 7,858,849, and 8,878,007; a Cry1Ac mutant of U.S. Pat. No. 9,512,187; a DIG-3 or DIG-11 toxin (N-terminal deletion of α-helix 1 and/or α-helix 2 variants of cry proteins such as Cry1A, Cry3A) of U.S. Pat. Nos. 8,304,604, 8.304,605 and 8,476,226; Cry1B of U.S. patent application Ser. No. 10/525,318, US Patent Application Publication Number US20160194364, and U.S. Pat. Nos. 9,404,121 and 8,772,577; Cry1B variants of PCT Publication Number WO2016/61197 and Serial Number PCT/US17/27160; Cry1C of U.S. Pat. No. 6,033,874; Cry1D protein of US20170233759; a Cry1E protein of PCT Serial Number PCT/US17/53178; a Cry1F protein of U.S. Pat. Nos. 5,188,960 and 6,218,188; Cry1A/F chimeras of U.S. Pat. Nos. 7,070,982; 6,962,705 and 6,713,063; a Cry1I protein of PCT Publication number WO 2017/0233759; a Cry1J variant of US Publication US20170240603; a Cry2 protein such as Cry2Ab protein of U.S. Pat. No. 7,064,249 and Cry2A.127 protein of U.S. Pat. No. 7,208,474; a Cry3A protein including but not limited to an engineered hybrid insecticidal protein (eHIP) created by fusing unique combinations of variable regions and conserved blocks of at least two different Cry proteins (US Patent Application Publication Number 2010/0017914); a Cry4 protein; a Cry5 protein; a Cry6 protein; Cry8 proteins of U.S. Pat. Nos. 7,329,736, 7,449,552, 7,803,943, 7,476,781, 7,105,332, 7,339,092, 7,378,499, 7,462,760, and 9,593,345; a Cry9 protein such as such as members of the Cry9A, Cry9B, Cry9C, Cry9D, Cry9E and Cry9F families including the Cry9 protein of U.S. Pat. Nos. 9,000,261 and 8,802,933, and US Serial Number WO 2017/132188; a Cry15 protein of Naimov, et al., (2008) Applied and Environmental Microbiology, 74:7145-7151; a Cry14 protein of U.S. Pat. No. 8,933,299; a Cry22, a Cry34Ab1 protein of U.S. Pat. Nos. 6,127,180, 6,624,145 and 6,340,593; a truncated Cry34 protein of U.S. Pat. No. 8,816,157; a CryET33 and cryET34 protein of U.S. Pat. Nos. 6,248,535, 6,326,351, 6,399,330, 6,949,626, 7,385,107 and 7,504,229; a CryET33 and CryET34 homologs of US Patent Publication Number 2006/0191034, 2012/0278954, and PCT Publication Number WO 2012/139004; a Cry35Ab1 protein of U.S. Pat. Nos. 6,083,499, 6,548,291 and 6,340,593; a Cry46 protein of U.S. Pat. No. 9,403,881, a Cry 51 protein, a Cry binary toxin; a TIC901 or related toxin; TIC807 of US Patent Application Publication Number 2008/0295207; TIC853 of U.S. Pat. No. 8,513,493; ET29, ET37, TIC809, TIC810, TIC812, TIC127, TIC128 of PCT US 2006/033867; engineered Hemipteran toxic proteins of US Patent Application Publication Number US20160150795, AXMI-027, AXMI-036, and AXMI-038 of U.S. Pat. No. 8,236,757; AXMI-031, AXMI-039, AXMI-040, AXMI-049 of U.S. Pat. No. 7,923,602; AXMI-018, AXMI-020 and AXMI-021 of WO 2006/083891; AXMI-010 of WO 2005/038032; AXMI-003 of WO 2005/021585; AXMI-008 of US Patent Application Publication Number 2004/0250311; AXMI-006 of US Patent Application Publication Number 2004/0216186; AXMI-007 of US Patent Application Publication Number 2004/0210965; AXMI-009 of US Patent Application Number 2004/0210964; AXMI-014 of US Patent Application Publication Number 2004/0197917; AXMI-004 of US Patent Application Publication Number 2004/0197916; AXMI-028 and AXMI-029 of WO 2006/119457; AXMI-007, AXMI-008, AXMI-0080rf2, AXMI-009, AXMI-014 and AXMI-004 of WO 2004/074462; AXMI-150 of U.S. Pat. No. 8,084,416; AXMI-205 of US Patent Application Publication Number 2011/0023184; AXMI-011, AXMI-012, AXMI-013, AXMI-015, AXMI-019, AXMI-044, AXMI-037, AXMI-043, AXMI-033, AXMI-034, AXMI-022, AXMI-023, AXMI-041, AXMI-063 and AXMI-064 of US Patent Application Publication Number 2011/0263488; AXMI046, AXMI048, AXMI050, AXMI051, AXMI052, AXMI053, AXMI054, AXMI055, AXMI056, AXMI057, AXMI058, AXMI059, AXMI060, AXMI061, AXMI067, AXMI069, AXMI071, AXMI072, AXMI073, AXMI074, AXMI075, AXMI087, AXMI088, AXMI093, AXMI070, AXMI080, AXMI081, AXMI082, AXMI091, AXMI092, AXMI096, AXMI097, AXMI098, AXMI099, AXMI100, AXMI101, AXMI102, AXMI103, AXMI104, AXMI107, AXMI108, AXMI109, AXMI110, AXMI111, AXMI112, AXMI114, AXMI116, AXMI117, AXMI118, AXMI119, AXMI120, AXMI121, AXMI122, AXMI123, AXMI124, AXMI125, AXMI126, AXMI127, AXMI129, AXMI151, AXMI161, AXMI164, AXMI183, AXMI132, AXMI137, AXMI138 of U.S. Pat. Nos. 8,461,421 and 8,461,422; AXMI-R1 and related proteins of US Patent Application Publication Number 2010/0197592; AXMI221Z, AXMI222z, AXMI223z, AXMI224z and AXMI225z of WO 2011/103248; AXMI218, AXMI219, AXMI220, AXMI226, AXMI227, AXMI228, AXMI229, AXMI230 and AXMI231 of WO 2011/103247; AXMI-115, AXMI-113, AXMI-005, AXMI-163 and AXMI-184 of U.S. Pat. No. 8,334,431; AXMI-001, AXMI-002, AXMI-030, AXMI-035 and AXMI-045 of US Patent Application Publication Number 2010/0298211; AXMI-066 and AXMI-076 of US Patent Application Publication Number 2009/0144852; AXMI128, AXMI130, AXMI131, AXMI133, AXMI140, AXMI141, AXMI142, AXMI143, AXMI144, AXMI146, AXMI148, AXMI149, AXMI152, AXMI153, AXMI154, AXMI155, AXMI156, AXMI157, AXMI158, AXMI162, AXMI165, AXMI166, AXMI167, AXMI168, AXMI169, AXMI170, AXMI171, AXMI172, AXMI173, AXMI174, AXMI175, AXMI176, AXMI177, AXMI178, AXMI179, AXMI180, AXMI181, AXMI182, AXMI185, AXMI186, AXMI187, AXMI188, AXMI189 of U.S. Pat. No. 8,318,900; AXMI079, AXMI080, AXMI081, AXMI082, AXMI091, AXMI092, AXMI096, AXMI097, AXMI098, AXMI099, AXMI100, AXMI101, AXMI102, AXMI103, AXMI104, AXMI107, AXMI108, AXMI109, AXMI110, dsAXMI111, AXMI112, AXMI114, AXMI116, AXMI117, AXMI118, AXMI119, AXMI120, AXMI121, AXMI122, AXMI123, AXMI124, AXMI1257, AXMI1268, AXMI127, AXMI129, AXMI164, AXMI151, AXMI161, AXMI183, AXMI132, AXMI138, AXMI137 of U.S. Pat. No. 8,461,421; AXMI192 of U.S. Pat. No. 8,461,415; AXMI281 of US Patent Application Publication Number US20160177332; AXMI422 of U.S. Pat. No. 8,252,872; cry proteins such as Cry1A and Cry3A having modified proteolytic sites of U.S. Pat. No. 8,319,019; a Cry1Ac, Cry2Aa and Cry1Ca toxin protein from Bacillus thuringiensis strain VBTS 2528 of US Patent Application Publication Number 2011/0064710. The Cry proteins MP032, MP049, MP051, MP066, MP068, MP070, MP091S, MP109S, MP114, MP121, MP134S, MP183S, MP185S, MP186S, MP195S, MP197S, MP208S, MP209S, MP212S, MP214S, MP217S, MP222S, MP234S, MP235S, MP237S, MP242S, MP243, MP248, MP249S, MP251M, MP252S, MP253, MP259S, MP287S, MP288S, MP295S, MP296S, MP297S, MP300S, MP304S, MP306S, MP310S, MP312S, MP314S, MP319S, MP325S, MP326S, MP327S, MP328S, MP334S, MP337S, MP342S, MP349S, MP356S, MP359S, MP360S, MP437S, MP451S, MP452S, MP466S, MP468S, MP476S, MP482S, MP522S, MP529S, MP548S, MP552S, MP562S, MP564S, MP566S, MP567S, MP569S, MP573S, MP574S, MP575S, MP581S, MP590, MP594S, MP596S, MP597, MP599S, MP600S, MP601S, MP602S, MP604S, MP626S, MP629S, MP630S, MP631S, MP632S, MP633S, MP634S, MP635S, MP639S, MP640S, MP644S, MP649S, MP651S, MP652S, MP653S, MP661S, MP666S, MP672S, MP696S, MP704S, MP724S, MP729S, MP739S, MP755S, MP773S, MP799S, MP800S, MP801S, MP802S, MP803S, MP805S, MP809S, MP815S, MP828S, MP831S, MP844S, MP852, MP865S, MP879S, MP887S, MP891S, MP896S, MP898S, MP935S, MP968, MP989, MP993, MP997, MP1049, MP1066, MP1067, MP1080, MP1081, MP1200, MP1206, MP1233, and MP1311 of U.S. Ser. No. 62/607,372. The insecticidal activity of Cry proteins is well known to one skilled in the art (for review, see, van Frannkenhuyzen, (2009) J. Invert. Path. 101:1-16). The use of Cry proteins as transgenic plant traits is well known to one skilled in the art and Cry-transgenic plants including but not limited to plants expressing Cry1Ac, Cry1Ac+Cry2Ab, Cry1Ab, Cry1A.105, Cry1F, Cry1Fa2, Cry1F+Cry1Ac, Cry2Ab, Cry3A, mCry3A, Cry3Bb1, Cry34Ab1, Cry35Ab1, Vip3A, mCry3A, Cry9c and CBI-Bt have received regulatory approval (see, Sanahuja, (2011) Plant Biotech Journal 9:283-300 and the CERA. (2010) GM Crop Database Center for Environmental Risk Assessment (CERA), ILSI Research Foundation, Washington D.C. at cera-gmc.org/index.php?action=gm_crop_database which can be accessed on the world-wide web using the “www” prefix). More than one pesticidal proteins well known to one skilled in the art can also be expressed in plants such as Vip3Ab & Cry1Fa (US2012/0317682); Cry1BE & Cry1F (US2012/0311746); Cry1CA & Cry1AB (US2012/0311745); Cry1F & CryCa (US2012/0317681); Cry1DA & Cry1BE (US2012/0331590); Cry1DA & Cry1Fa (US2012/0331589); Cry1AB & Cry1BE (US2012/0324606); Cry1Fa & Cry2Aa and Cry1I & Cry1E (US2012/0324605); Cry34Ab/35Ab & Cry6Aa (US20130167269); Cry34Ab/VCry35Ab & Cry3Aa (US20130167268); Cry1Da & Cry1Ca (U.S. Pat. No. 9,796,982); Cry3Aa & Cry6Aa (U.S. Pat. No. 9,798,963); and Cry3A & Cry1Ab or Vip3Aa (U.S. Pat. No. 9,045,766). Pesticidal proteins also include insecticidal lipases including lipid acyl hydrolases of U.S. Pat. No. 7,491,869, and cholesterol oxidases such as from Streptomyces (Purcell et al. (1993) Biochem Biophys Res Commun 15:1406-1413). Pesticidal proteins also include VIP (vegetative insecticidal proteins) toxins of U.S. Pat. Nos. 5,877,012, 6,107,279 6,137,033, 7,244,820, 7,615,686, and 8,237,020 and the like. Other VIP proteins are well known to one skilled in the art (see, lifesci.sussex.ac.uk/home/Neil_Crickmore/Bt/vip.html which can be accessed on the world-wide web using the “www” prefix). Pesticidal proteins also include Cyt proteins including Cyt1A variants of PCT Serial Number PCT/US2017/000510; Pesticidal proteins also include toxin complex (TC) proteins, obtainable from organisms such as Xenorhabdus, Photorhabdus and Paenibacillus (see, U.S. Pat. Nos. 7,491,698 and 8,084,418). Some TC proteins have “stand alone” insecticidal activity and other TC proteins enhance the activity of the stand-alone toxins produced by the same given organism. The toxicity of a “stand-alone” TC protein (from Photorhabdus, Xenorhabdus or Paenibacillus, for example) can be enhanced by one or more TC protein “potentiators” derived from a source organism of a different genus. There are three main types of TC proteins. As referred to herein, Class A proteins (“Protein A”) are stand-alone toxins. Class B proteins (“Protein B”) and Class C proteins (“Protein C”) enhance the toxicity of Class A proteins. Examples of Class A proteins are TcbA, TcdA, XptA1 and XptA2. Examples of Class B proteins are TcaC, TcdB, XptB1Xb and XptC1Wi. Examples of Class C proteins are TccC, XptC1Xb and XptB1Wi. Pesticidal proteins also include spider, snake and scorpion venom proteins. Examples of spider venom peptides include but not limited to lycotoxin-1 peptides and mutants thereof (U.S. Pat. No. 8,334,366). The combinations generated can also include multiple copies of any one of the polynucleotides of interest.

Transgenes that confer resistance to a herbicide, for Example: A polynucleotide encoding resistance to a herbicide that inhibits the growing point or meristem, such as an imidazolinone or a sulfonylurea. Exemplary genes in this category code for mutant ALS and AHAS enzyme as described, for example, by Lee, et al., (1988) EMBO J. 7:1241 and Miki, et al., (1990) Theor. Appl. Genet. 80:449, respectively. See also, U.S. Pat. Nos. 5,605,011; 5,013,659; 5,141,870; 5,767,361; 5,731,180; 5,304,732; 4,761,373; 5,331,107; 5,928,937 and 5,378,824; U.S. patent application Ser. No. 11/683,737 and International Publication WO 1996/33270. A polynucleotide encoding a protein for resistance to Glyphosate (resistance imparted by mutant 5-enolpyruvl-3-phosphikimate synthase (EPSP) and aroA genes, respectively) and other phosphono compounds such as glufosinate (phosphinothricin acetyl transferase (PAT) and Streptomyces hygroscopicus phosphinothricin acetyl transferase (bar) genes), and pyridinoxy or phenoxy proprionic acids and cyclohexones (ACCase inhibitor-encoding genes). See, for example, U.S. Pat. No. 4,940,835 to Shah, et al., which discloses the nucleotide sequence of a form of EPSPS which can confer glyphosate resistance. U.S. Pat. No. 5,627,061 to Barry, et al., also describes genes encoding EPSPS enzymes. See also, U.S. Pat. Nos. 6,566,587; 6,338,961; 6,248,876; 6,040,497; 5,804,425; 5,633,435; 5,145,783; 4,971,908; 5,312,910; 5,188,642; 5,094,945, 4,940,835; 5,866,775; 6,225,114; 6,130,366; 5,310,667; 4,535,060; 4,769,061; 5,633,448; 5,510,471; Re. 36,449; RE 37,287 E and 5,491,288 and International Publications EP 1173580; WO 2001/66704; EP 1173581 and EP 1173582, which are incorporated herein by reference for this purpose. Glyphosate resistance is also imparted to plants that express a gene encoding a glyphosate oxido-reductase enzyme as described more fully in U.S. Pat. Nos. 5,776,760 and 5,463,175, which are incorporated herein by reference for this purpose. In addition, glyphosate resistance can be imparted to plants by the over expression of genes encoding glyphosate N-acetyltransferase. See, for example, U.S. Pat. Nos. 7,462,481; 7,405,074 and US Patent Application Publication Number US 2008/0234130.

In some embodiments, the stacked trait may be in the form of silencing of one or more polynucleotides of interest resulting in suppression of one or more target pest polypeptides. In some embodiments, the silencing is achieved using a suppression DNA construct include, without limitation, cosuppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as siRNA (short interfering RNA) constructs and miRNA (microRNA) constructs.

In some embodiments, the stacked trait may be selected from events with regulatory approval which can be found at the Center for Environmental Risk Assessment (cera-gmc.org/?action=gm_crop_database, which can be accessed using the www prefix) and at the International Service for the Acquisition of Agri-Biotech Applications isaaa.org/gmapprovaldatabase/default.asp, which can be accessed using the www prefix).

### Use in Pesticidal Control

General methods for employing strains comprising a nucleic acid sequence of the embodiments or a variant thereof, in pesticide control or in engineering other organisms as pesticidal agents can be used.

Microorganism hosts that are known to occupy the “phytosphere” (phylloplane, phyllosphere, rhizosphere, and/or rhizoplana) of one or more crops of interest may be selected. These microorganisms are selected to be capable of successfully competing in the particular environment with the wild-type microorganisms, provide for stable maintenance and expression of the gene expressing the polypeptide of the disclosure and desirably provide for improved protection of the pesticide from environmental degradation and inactivation.

Alternatively, the polypeptide of the disclosure is produced by introducing a heterologous gene into a cellular host. Expression of the heterologous gene results, directly or indirectly, in the intracellular production and maintenance of the pesticide. These cells are then treated under conditions that prolong the activity of the toxin produced in the cell when the cell is applied to the environment of target pest(s). The resulting product retains the toxicity of the toxin. These naturally encapsulated polypeptides of the disclosure may then be formulated in accordance with conventional techniques for application to the environment hosting a target pest, e.g., soil, water, and foliage of plants. See, for example EPA 0192319, and the references cited therein.

### Pesticidal Compositions

In some embodiments, the active ingredients can be applied in the form of compositions and can be applied to the crop area or plant to be treated, simultaneously or in succession, with other compounds. These compounds can be fertilizers, weed killers, Cryoprotectants, surfactants, detergents, pesticidal soaps, dormant oils, polymers, and/or time-release or biodegradable carrier formulations that permit long-term dosing of a target area following a single application of the formulation. They can also be selective herbicides, chemical insecticides, virucides, microbicides, amoebicides, pesticides, fungicides, bacteriocides, nematocides, molluscicides or mixtures of several of these preparations, if desired, together with further agriculturally acceptable carriers, surfactants or application-promoting adjuvants customarily employed in the art of formulation. Suitable carriers and adjuvants can be solid or liquid and correspond to the substances ordinarily employed in formulation technology, e.g. natural or regenerated mineral substances, solvents, dispersants, wetting agents, tackifiers, binders or fertilizers. Likewise, the formulations may be prepared into edible “baits” or fashioned into pest “traps” to permit feeding or ingestion by a target pest of the pesticidal formulation.

Methods of applying an active ingredient or an agrochemical composition that contains at least one of the polypeptide of the disclosure produced by the bacterial strains include leaf application, seed coating and soil application. The number of applications and the rate of application depend on the intensity of infestation by the corresponding pest.

The composition may be formulated as a powder, dust, pellet, granule, spray, emulsion, colloid, solution or such like, and may be prepared by such conventional means as desiccation, lyophilization, homogenation, extraction, filtration, centrifugation, sedimentation or concentration of a culture of cells comprising the polypeptide. In all such compositions that contain at least one such pesticidal polypeptide, the polypeptide may be present in a concentration of from about 1% to about 99% by weight.

Lepidopteran, Dipteran, Heteropteran, nematode, Hemiptera or Coleopteran pests may be killed or reduced in numbers in a given area by the methods of the disclosure or may be prophylactically applied to an environmental area to prevent infestation by a susceptible pest. Preferably the pest ingests or is contacted with, a pesticidally-effective amount of the polypeptide. “Pesticidally-effective amount” as used herein refers to an amount of the pesticide that can bring about death to at least one pest or to noticeably reduce pest growth, feeding or normal physiological development. This amount will vary depending on such factors as, for example, the specific target pests to be controlled, the specific environment, location, plant, crop or agricultural site to be treated, the environmental conditions and the method, rate, concentration, stability, and quantity of application of the pesticidally-effective polypeptide composition. The formulations may also vary with respect to climatic conditions, environmental considerations, and/or frequency of application and/or severity of pest infestation.

The pesticide compositions described may be made by formulating either the bacterial cell, Crystal and/or spore suspension or isolated protein component with the desired agriculturally-acceptable carrier. The compositions may be formulated prior to administration in an appropriate means such as lyophilized, freeze-dried, desiccated or in an aqueous carrier, medium or suitable diluent, such as saline or another buffer. The formulated compositions may be in the form of a dust or granular material or a suspension in oil (vegetable or mineral) or water or oil/water emulsions or as a wettable powder or in combination with any other carrier material suitable for agricultural application. Suitable agricultural carriers can be a solid or liquid. The term “agriculturally-acceptable carrier” covers all adjuvants, inert components, dispersants, surfactants, tackifiers, binders, etc. that are ordinarily used in pesticide formulation technology. The formulations may be mixed with one or more solid or liquid adjuvants and prepared by various means, e.g., by homogeneously mixing, blending and/or grinding the pesticidal composition with suitable adjuvants using conventional formulation techniques. Suitable formulations and application methods are described in US Patent Number 6,468,523, herein incorporated by reference. The plants can also be treated with one or more chemical compositions, including one or more herbicide, insecticides or fungicides. Exemplary chemical compositions include: Fruits/Vegetables Herbicides: Atrazine, Bromacil, Diuron, Glyphosate, Linuron, Metribuzin, Simazine, Trifluralin, Fluazifop, Glufosinate, Halo sulfuron Gowan, Paraquat, Propyzamide, Sethoxydim, Butafenacil, Halosulfuron, Indaziflam; Fruits/Vegetables Insecticides: Aldicarb, Bacillus thuriengiensis, Carbaryl, Carbofuran, Chlorpyrifos, Cypermethrin, Deltamethrin, Diazinon, Malathion, Abamectin, Cyfluthrin/beta-cyfluthrin, Esfenvalerate, Lam bda-cyhalothrin, Acequinocyl, Bifenazate, Methoxyfenozide, Novaluron, Chromafenozide, Thiacloprid, Dinotefuran, FluaCrypyrim, Tolfenpyrad, Clothianidin, Spirodiclofen, Gamma-cyhalothrin, Spiromesifen, Spinosad, Rynaxypyr, Cyazypyr, Spinoteram, Triflumuron, Spirotetramat, Imidacloprid, Flubendiamide, Thiodicarb, Metaflumizone, Sulfoxaflor, Cyflumetofen, Cyanopyrafen, Imidacloprid, Clothianidin, Thiamethoxam, Spinotoram, Thiodicarb, Flonicamid, Methiocarb, Emamectin-benzoate, Indoxacarb, Forthiazate, Fenamiphos, Cadusaphos, Pyriproxifen, Fenbutatin-oxid, Hexthiazox, Methomyl, 4-[[(6-Chlorpyridin-3-yl)methyl](2,2-difluorethyl)amino]furan-2(5H)-on; Fruits/Vegetables Fungicides: Carbendazim, Chlorothalonil, EBDCs, Sulphur, Thiophanate-methyl, Azoxystrobin, Cymoxanil, Fluazinam, Fosetyl, Iprodione, Kresoxim-methyl, Metalaxyl/mefenoxam, Trifloxystrobin, Ethaboxam, Iprovalicarb, Trifloxystrobin, Fenhexamid, Oxpoconazole fumarate, Cyazofamid, Fenamidone, Zoxamide, Zorvec™, Picoxystrobin, Pyraclostrobin, Cyflufenamid, Boscalid; Cereals Herbicides: Isoproturon, Bromoxynil, loxynil, Phenoxies, Chlorsulfuron, Clodinafop, Diclofop, Diflufenican, Fenoxaprop, Florasulam, Fluoroxypyr, Metsulfuron, Triasulfuron, Flucarbazone, lodosulfuron, Propoxycarbazone, Picolinafen, Mesosulfuron, Beflubutamid, Pinoxaden, Amidosulfuron, Thifensulfuron Methyl, Tribenuron, Flupyrsulfuron, Sulfosulfuron, Pyrasulfotole, Pyroxsulam, Flufenacet, Tralkoxydim, Pyroxasulfon; Cereals Fungicides: Carbendazim, Chlorothalonil, Azoxystrobin, Cyproconazole, Cyprodinil, Fenpropimorph, Epoxiconazole, Kresoxim-methyl, Quinoxyfen, Tebuconazole, Trifloxystrobin, Simeconazole, Picoxystrobin, Pyraclostrobin, Dimoxystrobin, Prothioconazole, Fluoxastrobin; Cereals Insecticides: Dimethoate, Lambda-cyhalthrin, Deltamethrin, alpha-Cypermethrin, β-cyfluthrin, Bifenthrin, Imidacloprid, Clothianidin, Thiamethoxam, Thiacloprid, Acetamiprid, Dinetofuran, Clorphyriphos, Metamidophos, Oxidemethon-methyl, Pirimicarb, Methiocarb; Maize Herbicides: Atrazine, Alachlor, Bromoxynil, Acetochlor, Dicamba, Clopyralid, (S-) Dimethenamid, Glufosinate, Glyphosate, Isoxaflutole, (S-)Metolachlor, Mesotrione, Nicosulfuron, Primisulfuron, Revulin Q®; in Rimsulfuron, Sulcotrione, Foramsulfuron, Topramezone, Tern botrione, Saflufenacil, Thiencarbazone, Flufenacet, Pyroxasulfon; Maize Insecticides: Carbofuran, Chlorpyrifos, Bifenthrin, Fipronil, Imidacloprid, Lambda-Cyhalothrin, Tefluthrin, Terbufos, Thiamethoxam, Clothianidin, Spiromesifen, Flubendiamide, Triflumuron, Rynaxypyr, Deltamethrin, Thiodicarb, β-Cyfluthrin, Cypermethrin, Bifenthrin, Lufenuron, Triflumoron, Tefluthrin,Tebupirimphos, Ethiprole, Cyazypyr, Thiacloprid, Acetamiprid, Dinetofuran, Avermectin, Methiocarb, Spirodiclofen, Spirotetramat; Maize Fungicides: Fenitropan, Thiram, Prothioconazole, Tebuconazole, Trifloxystrobin; Rice Herbicides: Butachlor, Propanil, Azimsulfuron, Bensulfuron, Cyhalofop, Daimuron, Fentrazamide, Imazosulfuron, Mefenacet, Oxaziclomefone, Pyrazosulfuron, Pyributicarb, Quinclorac, Thiobencarb, Indanofan, Flufenacet, Fentrazamide, Halosulfuron, Oxaziclomefone, Benzobicyclon, Pyriftalid, Penoxsulam, Bispyribac, Oxadiargyl, Ethoxysulfuron, Pretilachlor, Mesotrione, Tefuryltrione, Oxadiazone, Fenoxaprop, Pyrimisulfan; Rice Insecticides: Diazinon, Fenitrothion, Fenobucarb, Monocrotophos, Benfuracarb, Buprofezin, Dinotefuran, Fipronil, Imidacloprid, Isoprocarb, Thiacloprid, Chromafenozide, Thiacloprid, Dinotefuran, Clothianidin, Ethiprole, Flubendiamide, Rynaxypyr, Deltamethrin, Acetamiprid, Thiamethoxam, Cyazypyr, Spinosad, Spinotoram, Emamectin-Benzoate, Cypermethrin, Chlorpyriphos, Cartap, Methamidophos, Etofenprox, Triazophos, 4-[[(6-Chlorpyridin-3-yl)methyl](2,2-difluorethyl)amino]furan-2(5H)-on, Carbofuran, Benfuracarb; Rice Fungicides: Thiophanate-methyl, Azoxystrobin, Carpropamid, Edifenphos, Ferimzone, Iprobenfos, Isoprothiolane, Pencycuron, Probenazole, Pyroquilon, Tricyclazole, Trifloxystrobin, Diclocymet, Fenoxanil, Simeconazole, Tiadinil; Cotton Herbicides: Diuron, Fluometuron, MSMA, Oxyfluorfen, Prometryn, Trifluralin, Carfentrazone, Clethodim, Fluazifop-butyl, Glyphosate, Norflurazon, Pendimethalin, Pyrithiobac-sodium, Trifloxysulfuron, Tepraloxydim, Glufosinate, Flumioxazin, Thidiazuron; Cotton Insecticides: Acephate, Aldicarb, Chlorpyrifos, Cypermethrin, Deltamethrin, Malathion, Monocrotophos, Abamectin, Acetamiprid, Emamectin Benzoate, Imidacloprid, Indoxacarb, Lambda-Cyhalothrin, Spinosad, Thiodicarb, Gamma-Cyhalothrin, Spiromesifen, Pyridalyl, Flonicamid, Flubendiamide, Triflumuron, Rynaxypyr, Beta-Cyfluthrin, Spirotetramat, Clothianidin, Thiamethoxam, Thiacloprid, Dinetofuran, Flubendiamide, Cyazypyr, Spinosad, Spinotoram, gamma Cyhalothrin, 4-[[(6-Chlorpyridin-3-yl)methyl](2,2-difluorethyl)amino]furan-2(5H)-on, Thiodicarb, Avermectin, Flonicamid, Pyridalyl, Spiromesifen, Sulfoxaflor, Profenophos, Thriazophos, Endosulfan; Cotton Fungicides: Etridiazole, Metalaxyl, Quintozene; Soybean Herbicides: Alachlor, Bentazone, Trifluralin, Chlorimuron-Ethyl, Cloransulam-Methyl, Fenoxaprop, Fomesafen, Fluazifop, Glyphosate, Imazamox, Imazaquin, Imazethapyr, (S-Metolachlor, Metribuzin, Pendimethalin, Tepraloxydim, Glufosinate; Soybean Insecticides: Lambda-cyhalothrin, Methomyl, Parathion, Thiocarb, Imidacloprid, Clothianidin, Thiamethoxam, Thiacloprid, Acetamiprid, Dinetofuran, Flubendiamide, Rynaxypyr, Cyazypyr, Spinosad, Spinotoram, Emamectin-Benzoate, Fipronil, Ethiprole, Deltamethrin, β-Cyfluthrin, gamma and lambda Cyhalothrin, 4-[[(6-Chlorpyridin-3-yl)methyl](2,2-difluorethyl)amino]furan-2(5H)-on, Spirotetramat, Spinodiclofen, Triflumuron, Flonicamid, Thiodicarb, beta-Cyfluthrin; Soybean Fungicides: Azoxystrobin, Cyproconazole, Epoxiconazole, Flutriafol, Pyraclostrobin, Tebuconazole, Trifloxystrobin, Prothioconazole, Tetraconazole; Sugarbeet Herbicides: Chloridazon, Desmedipham, Ethofumesate, Phenmedipham, Triallate, Clopyralid, Fluazifop, Lenacil, Metamitron, Quinmerac, Cycloxydim, Triflusulfuron, Tepraloxydim, Quizalofop; Sugarbeet Insecticides: Imidacloprid, Clothianidin, Thiamethoxam, Thiacloprid, Acetamiprid, Dinetofuran, Deltamethrin, β-Cyfluthrin, gamma/lambda Cyhalothrin, 4-[[(6-Chlorpyridin-3-yl)methyl](2,2-difluorethyl)amino]furan-2(5H)-on, Tefluthrin, Rynaxypyr, Cyaxypyr, Fipronil, Carbofuran; Canola Herbicides: Clopyralid, Diclofop, Fluazifop, Glufosinate, Glyphosate, Metazachlor, Trifluralin Ethametsulfuron, Quinmerac, Quizalofop, Clethodim, Tepraloxydim; Canola Fungicides: Azoxystrobin, Carbendazim, Fludioxonil, Iprodione, Prochloraz, Vinclozolin; Canola Insecticides: Carbofuran organophosphates, Pyrethroids, Thiacloprid, Deltamethrin, Imidacloprid, Clothianidin, Thiamethoxam, Acetamiprid, Dinetofuran, β-Cyfluthrin, gamma and lambda Cyhalothrin, tau-Fluvaleriate, Ethiprole, Spinosad, Spinotoram, Flubendiamide, Rynaxypyr, Cyazypyr, 4-[[(6-Chlorpyridin-3-yl)methyl](2,2-difluorethyl)amino]furan-2(5H)-on.

In some embodiments, the herbicide is Atrazine, Bromacil, Diuron, Chlorsulfuron, Metsulfuron, Thifensulfuron Methyl, Tribenuron, Acetochlor, Dicamba, Isoxaflutole, Nicosulfuron, Rimsulfuron, Pyrithiobac-sodium, Flumioxazin, Chlorimuron-Ethyl, Metribuzin, Quizalofop, S-metolachlor, Hexazinne or combinations thereof.

In some embodiments, the insecticide is Esfenvalerate, Chlorantraniliprole, Methomyl, Indoxacarb, Oxamyl or combinations thereof.

### Pesticidal and Insecticidal Activity

“Pest” includes but is not limited to, insects, fungi, bacteria, nematodes, mites, ticks and the like. Insect pests include insects selected from the orders Coleoptera, Diptera, Hymenoptera, Lepidoptera, Mallophaga, Homoptera, Hemiptera Orthroptera, Thysanoptera, Dermaptera, Isoptera, Anoplura, Siphonaptera, Trichoptera, etc., particularly Lepidoptera and Coleoptera.

Those skilled in the art will recognize that not all compounds are equally effective against all pests. Compounds of the embodiments display activity against insect pests, which may include economically important agronomic, forest, greenhouse, nursery ornamentals, food and fiber, public and animal health, domestic and commercial structure, household and stored product pests.

Larvae of the order Lepidoptera include, but are not limited to, armyworms, cutworms, loopers and heliothines in the family Noctuidae Spodoptera frugiperda (fall armyworm); Spodoptera exigua Hübner (beet armyworm); Spodoptera litura Fabricius (tobacco cutworm, cluster caterpillar); Mamestra configurata Walker (bertha armyworm); Mamestra brassicae Linnaeus (cabbage moth); Agrotis ipsilon Hufnagel (black cutworm); Agrotis orthogonia Morrison (western cutworm); Agrotis subterranea Fabricius (granulate cutworm); Alabama argillacea Hübner (cotton leaf worm); Trichoplusia ni Hübner (cabbage looper); Pseudoplusia includens Walker (soybean looper); Anticarsia gemmatalis Hübner (velvetbean caterpillar); Hypena scabra Fabricius (green cloverworm); Heliothis virescens Fabricius (tobacco budworm); Pseudaletia unipuncta Haworth (armyworm); Athetis mindara Barnes and Mcdunnough (rough skinned cutworm); Euxoa messoria Harris (darksided cutworm); Earias insulana Boisduval (spiny bollworm); Earias vittella Fabricius (spotted bollworm); Helicoverpa armigera Hübner (American bollworm); H. zea Boddie (corn earworm or cotton bollworm); Melanchra picta Harris (zebra caterpillar); Egira (Xylomyges) curialis Grote (citrus cutworm); borers, casebearers, webworms, coneworms, and skeletonizers from the family Pyralidae Ostrinia nubilalis Hübner (European corn borer); Amyelois transitella Walker (naval orangeworm); Anagasta kuehniella Zeller (Mediterranean flour moth); Cadra cautella Walker (almond moth); Chilo suppressalis Walker (rice stem borer); Chilo partellus, (sorghum borer); Corcyra cephalonica Stainton (rice moth); Crambus caliginosellus Clemens (corn root webworm); Crambus teterrellus Zincken (bluegrass webworm); Cnaphalocrocis medinalis Guenée (rice leaf roller); Desmia funeralis Hübner (grape leaffolder); Diaphania hyalinata Linnaeus (melon worm); Diaphania nitidalis Stoll (pickleworm); Diatraea grandiosella Dyar (southwestern corn borer), Diatraea saccharalis Fabricius (surgarcane borer); Eoreuma loftini Dyar (Mexican rice borer); Ephestia elutella Hübner (tobacco (cacao) moth); Galleria mellonella Linnaeus (greater wax moth); Herpetogramma licarsisalis Walker (sod webworm); Homoeosoma electellum Hulst (sunflower moth); Elasmopalpus lignosellus Zeller (lesser cornstalk borer); Achroia grisella Fabricius (lesser wax moth); Loxostege sticticalis Linnaeus (beet webworm); Orthaga thyrisalis Walker (tea tree web moth); Maruca testulalis Geyer (bean pod borer); Plodia interpunctella Hübner (Indian meal moth); Scirpophaga incertulas Walker (yellow stem borer); Udea rubigalis Guenée (celery leaftier); and leafrollers, budworms, seed worms and fruit worms in the family Tortricidae Acleris gloverana Walsingham (Western blackheaded budworm); Acleris variana Fernald (Eastern blackheaded budworm); Archips argyrospila Walker (fruit tree leaf roller); Archips rosana Linnaeus (European leaf roller); and other Archips species, Adoxophyes orana Fischer von Rösslerstamm (summer fruit tortrix moth); Cochylis hospes Walsingham (banded sunflower moth); Cydia latiferreana Walsingham (filbertworm); Cydia pomonella Linnaeus (coding moth); Platynota flavedana Clemens (variegated leafroller); Platynota stultana Walsingham (omnivorous leafroller); Lobesia botrana Denis & Schiffermüller (European grape vine moth); Spilonota ocellana Denis & Schiffermüller (eyespotted bud moth); Endopiza viteana Clemens (grape berry moth); Eupoecilia ambiguella Hübner (vine moth); Bonagota salubricola Meyrick (Brazilian apple leafroller); Grapholita molesta Busck (oriental fruit moth); Suleima helianthana Riley (sunflower bud moth); Argyrotaenia spp.; Choristoneura spp.

Selected other agronomic pests in the order Lepidoptera include, but are not limited to, Alsophila pometaria Harris (fall cankerworm); Anarsia lineatella Zeller (peach twig borer); Anisota senatoria (orange striped oakworm); Antheraea pernyi Guérin-Méneville (Chinese Oak Tussah Moth); Bombyx mori Linnaeus (Silkworm); Bucculatrix thurberiella Busck (cotton leaf perforator); Colias eurytheme Boisduval (alfalfa caterpillar); Datana integerrima Grote & Robinson (walnut caterpillar); Dendrolimus sibiricus Tschetwerikov (Siberian silk moth), Ennomos subsignaria Hübner (elm spanworm); Erannis tiliaria Harris (linden looper); Euproctis chrysorrhoea Linnaeus (browntail moth); Harrisina americana Guérin-Méneville (grapeleaf skeletonizer); Hemileuca oliviae Cockrell (range caterpillar); Hyphantria cunea Drury (fall webworm); Keiferia lycopersicella Walsingham (tomato pinworm); Lambdina fiscellaria fiscellaria Hulst (Eastern hemlock looper); L. fiscellaria lugubrosa Hulst (Western hemlock looper); Leucoma salicis Linnaeus (satin moth); Lymantria dispar Linnaeus (gypsy moth); Manduca quinquemaculata Haworth (five spotted hawk moth, tomato hornworm); M. sexta Haworth (tomato hornworm, tobacco hornworm); Operophtera brumata Linnaeus (winter moth); Paleacrita vemata Peck (spring cankerworm); Papilio cresphontes Cramer (giant swallowtail orange dog); Phryganidia califomica Packard (California oakworm); Phyllocnistis citrella Stainton (citrus leafminer); Phyllonorycter blancardella Fabricius (spotted tentiform leafminer); Pieris brassicae Linnaeus (large white butterfly); P. rapae Linnaeus (small white butterfly); P. napi Linnaeus (green veined white butterfly); Platyptilia carduidactyla Riley (artichoke plume moth); Plutella xylostella Linnaeus (diamondback moth); Pectinophora gossypiella Saunders (pink bollworm); Pontia protodice Boisduval and Leconte (Southern cabbageworm); Sabulodes aegrotata Guenée (omnivorous looper); Schizura concinna J. E. Smith (red humped caterpillar); Sitotroga cerealella Olivier (Angoumois grain moth); Thaumetopoea pityocampa Schiffermuller (pine processionary caterpillar); Tineola bisselliella Hummel (webbing clothesmoth); Tuta absoluta Meyrick (tomato leafminer); Yponomeuta padella Linnaeus (ermine moth); Heliothis subflexa Guenée; Malacosoma spp. and Orgyia spp.

Of interest are larvae and adults of the order Coleoptera including weevils from the families Anthribidae, Bruchidae and Curculionidae (including, but not limited to: Anthonomus grandis Boheman (boll weevil); Lissorhoptrus oryzophilus Kuschel (rice water weevil); Sitophilus granarius Linnaeus (granary weevil); S. oryzae Linnaeus (rice weevil); Hypera punctata Fabricius (clover leaf weevil); Cylindrocopturus adspersus LeConte (sunflower stem weevil); Smicronyx fulvus LeConte (red sunflower seed weevil); S. sordidus LeConte (gray sunflower seed weevil); Sphenophorus maidis Chittenden (maize billbug)); flea beetles, cucumber beetles, rootworms, leaf beetles, potato beetles and leafminers in the family Chrysomelidae (including, but not limited to: Leptinotarsa decemlineata Say (Colorado potato beetle); Diabrotica virgifera virgifera LeConte (western corn rootworm); D. barberi Smith and Lawrence (northern corn rootworm); D. undecimpunctata howardi Barber (southern corn rootworm); Chaetocnema pulicaria Melsheimer (corn flea beetle); Phyllotreta cruciferae Goeze (Crucifer flea beetle); Phyllotreta striolata (stripped flea beetle); Colaspis brunnea Fabricius (grape colaspis); Oulema melanopus Linnaeus (cereal leaf beetle); Zygogramma exclamationis Fabricius (sunflower beetle)); beetles from the family Coccinellidae (including, but not limited to: Epilachna varivestis Mulsant (Mexican bean beetle)); chafers and other beetles from the family Scarabaeidae (including, but not limited to: Popillia japonica Newman (Japanese beetle); Cyclocephala borealis Arrow (northern masked chafer, white grub); C. immaculata Olivier (southern masked chafer, white grub); Rhizotrogus majalis Razoumowsky (European chafer); Phyllophaga crinita Burmeister (white grub); Ligyrus gibbosus De Geer (carrot beetle)); carpet beetles from the family Dermestidae; wireworms from the family Elateridae, Eleodes spp., Melanotus spp.; Conoderus spp.; Limonius spp.; Agriotes spp.; Ctenicera spp.; Aeolus spp.; bark beetles from the family Scolytidae and beetles from the family Tenebrionidae.

Adults and immatures of the order Diptera are of interest, including leafminers Agromyza parvicomis Loew (corn blotch leafminer); midges (including, but not limited to: Contarinia sorghicola Coquillett (sorghum midge); Mayetiola destructor Say (Hessian fly); Sitodiplosis mosellana Géhin (wheat midge); Neolasioptera murtfeldtiana Felt, (sunflower seed midge)); fruit flies (Tephritidae), Oscinella frit Linnaeus (fruit flies); maggots (including, but not limited to: Delia platura Meigen (seedcorn maggot); D. coarctata Fallen (wheat bulb fly) and other Delia spp., Meromyza americana Fitch (wheat stem maggot); Musca domestica Linnaeus (house flies); Fannia canicularis Linnaeus, F. femoralis Stein (lesser house flies); Stomoxys calcitrans Linnaeus (stable flies)); face flies, horn flies, blow flies, Chrysomya spp.; Phormia spp. and other muscoid fly pests, horse flies Tabanus spp.; bot flies Gastrophilus spp.; Oestrus spp.; cattle grubs Hypoderma spp.; deer flies Chrysops spp.; Melophagus ovinus Linnaeus (keds) and other Brachycera, mosquitoes Aedes spp.; Anopheles spp.; Culex spp.; black flies Prosimulium spp.; Simulium spp.; biting midges, sand flies, sciarids, and other Nematocera.

Included as insects of interest are adults and nymphs of the orders Hemiptera and Homoptera such as, but not limited to, adelgids from the family Adelgidae, plant bugs from the family Miridae, cicadas from the family Cicadidae, leafhoppers, Empoasca spp.; from the family Cicadellidae, planthoppers from the families Cixiidae, Flatidae, Fulgoroidea, lssidae and Delphacidae, treehoppers from the family Membracidae, psyllids from the family Psyllidae, whiteflies from the family Aleyrodidae, aphids from the family Aphididae, phylloxera from the family Phylloxeridae, mealybugs from the family Pseudococcidae, scales from the families Asterolecanidae, Coccidae, Dactylopiidae, Diaspididae, Eriococcidae Ortheziidae, Phoenicococcidae and Margarodidae, lace bugs from the family Tingidae, stink bugs from the family Pentatomidae, cinch bugs, Blissus spp.; and other seed bugs from the family Lygaeidae, spittlebugs from the family Cercopidae squash bugs from the family Coreidae and red bugs and cotton stainers from the family Pyrrhocoridae.

Agronomically important members from the order Homoptera further include, but are not limited to: Acyrthisiphon pisum Harris (pea aphid); Aphis craccivora Koch (cowpea aphid); A. fabae Scopoli (black bean aphid); A. gossypii Glover (cotton aphid, melon aphid); A. maidiradicis Forbes (corn root aphid); A. pomi De Geer (apple aphid); A. spiraecola Patch (spirea aphid); Aulacorthum solani Kaltenbach (foxglove aphid); Chaetosiphon fragaefolii Cockerell (strawberry aphid); Diuraphis noxia Kurdjumov/Mordvilko (Russian wheat aphid); Dysaphis plantaginea Paaserini (rosy apple aphid); Eriosoma lanigerum Hausmann (woolly apple aphid); Brevicoryne brassicae Linnaeus (cabbage aphid); Hyalopterus pruni Geoffroy (mealy plum aphid); Lipaphis erysimi Kaltenbach (turnip aphid); Metopolophium dirrhodum Walker (cereal aphid); Macrosiphum euphorbiae Thomas (potato aphid); Myzus persicae Sulzer (peach-potato aphid, green peach aphid); Nasonovia ribisnigri Mosley (lettuce aphid); Pemphigus spp. (root aphids and gall aphids); Rhopalosiphum maidis Fitch (corn leaf aphid); R. padi Linnaeus (bird cherry-oat aphid); Schizaphis graminum Rondani (greenbug); Sipha flava Forbes (yellow sugarcane aphid); Sitobion avenae Fabricius (English grain aphid); Therioaphis maculata Buckton (spotted alfalfa aphid); Toxoptera aurantii Boyer de Fonscolombe (black citrus aphid) and T. citricida Kirkaldy (brown citrus aphid); Adelges spp. (adelgids); Phylloxera devastatrix Pergande (pecan phylloxera); Bemisia tabaci Gennadius (tobacco whitefly, sweetpotato whitefly); B. argentifolii Bellows & Perring (silverleaf whitefly); Dialeurodes citri Ashmead (citrus whitefly); Trialeurodes abutiloneus (bandedwinged whitefly) and T. vaporariorum Westwood (greenhouse whitefly); Empoasca fabae Harris (potato leafhopper); Laodelphax striatellus Fallen (smaller brown planthopper); Macrolestes quadrilineatus Forbes (aster leafhopper); Nephotettix cinticeps Uhler (green leafhopper); N. nigropictus StåI (rice leafhopper); Nilaparvata lugens StåI (brown planthopper); Peregrinus maidis Ashmead (corn planthopper); Sogatella furcifera Horvath (white-backed planthopper); Sogatodes orizicola Muir (rice delphacid); Typhlocyba pomaria McAtee (white apple leafhopper); Erythroneoura spp. (grape leafhoppers); Magicicada septendecim Linnaeus (periodical cicada); Icerya purchasi Maskell (cottony cushion scale); Quadraspidiotus perniciosus Comstock (San Jose scale); Planococcus citri Risso (citrus mealybug); Pseudococcus spp. (other mealybug complex); Cacopsylla pyricola Foerster (pear psylla); Trioza diospyri Ashmead (persimmon psylla).

Agronomically important species of interest from the order Hemiptera include, but are not limited to: Acrosternum hilare Say (green stink bug); Anasa tristis De Geer (squash bug); Blissus leucopterus leucopterus Say (chinch bug); Corythuca gossypii Fabricius (cotton lace bug); Cyrtopeltis modesta Distant (tomato bug); Dysdercus suturellus Herrich-Schäffer (cotton stainer); Euschistus servus Say (brown stink bug); E. variolarius Palisot de Beauvois (one-spotted stink bug); Graptostethus spp. (complex of seed bugs); Leptoglossus corculus Say (leaf-footed pine seed bug); Lygus lineolaris Palisot de Beauvois (tarnished plant bug); L. hesperus Knight (Western tarnished plant bug); L. pratensis Linnaeus (common meadow bug); L. rugulipennis Poppius (European tarnished plant bug); Lygocoris pabulinus Linnaeus (common green capsid); Nezara viridula Linnaeus (southern green stink bug); Oebalus pugnax Fabricius (rice stink bug); Oncopeltus fasciatus Dallas (large milkweed bug); Pseudatomoscelis seriatus Reuter (cotton fleahopper).

Furthermore, embodiments may be effective against Hemiptera such, Calocoris norvegicus Gmelin (strawberry bug); Orthops campestris Linnaeus; Plesiocoris rugicollis Fallen (apple capsid); CyrtopeIlls modestus Distant (tomato bug); CyrtopeIlls notatus Distant (suckfly); Spanagonicus albofasciatus Reuter (whitemarked fleahopper); Diaphnocoris chlorionis Say (honeylocust plant bug); Labopidicola allii Knight (onion plant bug); Pseudatomoscelis seriatus Reuter (cotton fleahopper); Adelphocoris rapidus Say (rapid plant bug); Poecilocapsus lineatus Fabricius (four-lined plant bug); Nysius ericae Schilling (false chinch bug); Nysius raphanus Howard (false chinch bug); Nezara viridula Linnaeus (Southern green stink bug); Eurygaster spp.; Coreidae spp.; Pyrrhocoridae spp.; Tinidae spp.; Blostomatidae spp.; Reduviidae spp. and Cimicidae spp.

Also included are adults and larvae of the order Acari (mites) such as Aceria tosichella Keifer (wheat curl mite); Petrobia latens Müller (brown wheat mite); spider mites and red mites in the family Tetranychidae, Panonychus ulmi Koch (European red mite); Tetranychus urticae Koch (two spotted spider mite); (T. mcdanieli McGregor (McDaniel mite); T. cinnabarinus Boisduval (carmine spider mite); T. turkestani Ugarov & Nikolski (strawberry spider mite); flat mites in the family Tenuipalpidae, Brevipalpus lewisi McGregor (citrus flat mite); rust and bud mites in the family Eriophyidae and other foliar feeding mites and mites important in human and animal health, i.e., dust mites in the family Epidermoptidae, follicle mites in the family Demodicidae, and grain mites in the family Glycyphagidae.

Insect pest of interest include the superfamily of stink bugs and other related insects including but not limited to species belonging to the family Pentatomidae (Nezara viridula, Halyomorpha halys, Piezodorus guildini, Euschistus servus, Acrosternum hilare, Euschistus heros, Euschistus tristigmus, Acrosternum hilare, Dichelops furcatus, Dichelops melacanthus, and Bagrada hilaris (Bagrada Bug)), the family Plataspidae (Megacopta cribraria—Bean plataspid) and the family Cydnidae (Scaptocoris castanea—Root stink bug) and Lepidoptera species including but not limited to: diamond-back moth, e.g., Helicoverpa zea Boddie; soybean looper, e.g., Pseudoplusia includens Walker and velvet bean caterpillar e.g., Anticarsia gemmatalis Hübner.

Methods for measuring pesticidal activity can be found in Czapla and Lang, (1990) J. Econ. Entomol. 83:2480-2485; Andrews, et al., (1988) Biochem. J. 252:199-206; Marrone, et al., (1985) J. of Economic Entomology 78:290-293 and U.S. Pat. No. 5,743,477, all of which are herein incorporated by reference in their entirety. Generally, the protein is mixed and used in feeding assays. See, for example Marrone, et al., (1985) J. of Economic Entomology 78:290-293. Such assays can include contacting plants with one or more pests and determining the plant's ability to survive and/or cause the death of the pests.

Nematodes include parasitic nematodes such as root-knot, cyst and lesion nematodes, including Heterodera spp., Meloidogyne spp. and Globodera spp.; particularly members of the cyst nematodes, including, but not limited to, Heterodera glycines (soybean cyst nematode); Heterodera schachtii (beet cyst nematode); Heterodera avenae (cereal cyst nematode) and Globodera rostochiensis and Globodera pailida (potato cyst nematodes). Lesion nematodes include Pratylenchus spp.

### Seed Treatment

To protect and to enhance yield production and trait technologies, seed treatment options can provide additional crop plan flexibility and cost effective control against insects, weeds and diseases. Seed material can be treated, typically surface treated, with a composition comprising combinations of chemical or biological herbicides, herbicide safeners, insecticides, fungicides, germination inhibitors and enhancers, nutrients, plant growth regulators and activators, bactericides, nematocides, avicides and/or molluscicides. These compounds are typically formulated together with further carriers, surfactants or application-promoting adjuvants customarily employed in the art of formulation. The coatings may be applied by impregnating propagation material with a liquid formulation or by coating with a combined wet or dry formulation. Examples of the various types of compounds that may be used as seed treatments are provided in The Pesticide Manual: A World Compendium, C.D.S. Tomlin Ed., Published by the British Crop Production Council, which is hereby incorporated by reference.

Some seed treatments that may be used on crop seed include, but are not limited to, one or more of abscisic acid, acibenzolar-S-methyl, avermectin, amitrol, azaconazole, azospirillum, azadirachtin, azoxystrobin, Bacillus spp. (including one or more of cereus, firmus, megaterium, pumilis, sphaericus, subtilis and/or thuringiensis species), Bradyrhizobium spp. (including one or more of betae, canariense, elkanii, iriomotense, japonicum, liaonigense, pachyrhizi and/or yuanmingense), captan, carboxin, chitosan, clothianidin, copper, cyazypyr, difenoconazole, etidiazole, fipronil, fludioxonil, fluoxastrobin, fluquinconazole, flurazole, fluxofenim, harpin protein, imazalil, imidacloprid, ipconazole, isoflavenoids, lipo-chitooligosaccharide, mancozeb, manganese, maneb, mefenoxam, metalaxyl, metconazole, myclobutanil, PCNB, penflufen, penicillium, penthiopyrad, permethrine, picoxystrobin, prothioconazole, pyraclostrobin, rynaxypyr, S-metolachlor, saponin, sedaxane, TCMTB, tebuconazole, thiabendazole, thiamethoxam, thiocarb, thiram, tolclofos-methyl, triadimenol, trichoderma, trifloxystrobin, triticonazole and/or zinc. PCNB seed coat refers to EPA Registration Number 00293500419, containing quintozen and terrazole. TCMTB refers to 2-(thiocyanomethylthio) benzothiazole.

Seed varieties and seeds with specific transgenic traits may be tested to determine which seed treatment options and application rates may complement such varieties and transgenic traits to enhance yield. For example, a variety with good yield potential but head smut susceptibility may benefit from the use of a seed treatment that provides protection against head smut, a variety with good yield potential but cyst nematode susceptibility may benefit from the use of a seed treatment that provides protection against cyst nematode, and so on. Likewise, a variety encompassing a transgenic trait conferring insect resistance may benefit from the second mode of action conferred by the seed treatment, a variety encompassing a transgenic trait conferring herbicide resistance may benefit from a seed treatment with a safener that enhances the plants resistance to that herbicide, etc. Further, the good root establishment and early emergence that results from the proper use of a seed treatment may result in more efficient nitrogen use, a better ability to withstand drought and an overall increase in yield potential of a variety or varieties containing a certain trait when combined with a seed treatment.

### Methods for Killing an Insect Pest and Controlling an Insect Population

In some embodiments methods are provided for killing an insect pest, comprising contacting the insect pest, either simultaneously or sequentially, with an insecticidally-effective amount of a recombinant polypeptide of the disclosure.

In some embodiments methods are provided for controlling an insect pest population, comprising contacting the insect pest population, either simultaneously or sequentially, with an insecticidally-effective amount of a recombinant polypeptide of the disclosure. As used herein, “controlling a pest population” or “controls a pest” refers to any effect on a pest that results in limiting the damage that the pest causes. Controlling a pest includes, but is not limited to, killing the pest, inhibiting development of the pest, altering fertility or growth of the pest in such a manner that the pest provides less damage to the plant, decreasing the number of offspring produced, producing less fit pests, producing pests more susceptible to predator attack or deterring the pests from eating the plant.

In some embodiments methods are provided for controlling an insect pest population resistant to a pesticidal protein, comprising contacting the insect pest population, either simultaneously or sequentially, with an insecticidally-effective amount of a recombinant polypeptide of the disclosure.

In some embodiments methods are provided for protecting a plant from an insect pest, comprising expressing in the plant or cell thereof at least one recombinant polynucleotide encoding a polypeptide of the disclosure.

In some embodiments methods are provided for protecting a plant from an insect pest, comprising expressing in the plant or cell thereof a recombinant polynucleotide encoding a polypeptide of the disclosure.

### Insect Resistance Management (IRM) Strategies

Expression of B. thuringiensis δ-endotoxins in transgenic corn plants has proven to be an effective means of controlling agriculturally important insect pests (Perlak, et al., 1990; 1993). However, insects have evolved that are resistant to B. thuringiensis δ-endotoxins expressed in transgenic plants. Such resistance, should it become widespread, would clearly limit the commercial value of germplasm containing genes encoding such B. thuringiensis δ-endotoxins.

One way to increasing the effectiveness of the transgenic insecticides against target pests and contemporaneously reducing the development of insecticide-resistant pests is to use provide non-transgenic (i.e., non-insecticidal protein) refuges (a section of non-insecticidal crops/corn) for use with transgenic crops producing a single insecticidal protein active against target pests. The United States Environmental Protection Agency (epa.gov/oppbppdl/biopesticides/pips/bt_corn_refuge_2006.htm, which can be accessed using the www prefix) publishes the requirements for use with transgenic crops producing a single Bt protein active against target pests. In addition, the National Corn Growers Association, on their website: (ncga.com/insect-resistance-management-fact-sheet-bt-corn, which can be accessed using the www prefix) also provides similar guidance regarding refuge requirements. Due to losses to insects within the refuge area, larger refuges may reduce overall yield.

Another way of increasing the effectiveness of the transgenic insecticides against target pests and contemporaneously reducing the development of insecticide-resistant pests would be to have a repository of insecticidal genes that are effective against groups of insect pests and which manifest their effects through different modes of action.

Expression in a plant of two or more insecticidal compositions toxic to the same insect species, each insecticide being expressed at efficacious levels would be another way to achieve control of the development of resistance. This is based on the principle that evolution of resistance against two separate modes of action is far more unlikely than only one. Roush, for example, outlines two-toxin strategies, also called “pyramiding” or “stacking,” for management of insecticidal transgenic crops. (The Royal Society. Phil. Trans. R. Soc. Lond. B. (1998) 353:1777-1786). Stacking or pyramiding of two different proteins each effective against the target pests and with little or no cross-resistance can allow for use of a smaller refuge. The US Environmental Protection Agency requires significantly less (generally 5%) structured refuge of non-Bt corn be planted than for single trait products (generally 20%). There are various ways of providing the IRM effects of a refuge, including various geometric planting patterns in the fields and in-bag seed mixtures, as discussed further by Roush.

In some embodiments, the polypeptides of the disclosure are useful as an insect resistance management strategy in combination (i.e., pyramided) with other pesticidal proteins include but are not limited to Bt toxins, Xenorhabdus sp. or Photorhabdus sp. insecticidal proteins, other insecticidally active proteins, and the like.

Provided are methods of controlling Lepidoptera and/or Coleoptera insect infestation(s) in a transgenic plant that promote insect resistance management, comprising expressing in the plant at least two different insecticidal proteins having different modes of action.

In some embodiments, the methods of controlling Lepidoptera and/or Coleoptera insect infestation in a transgenic plant and promoting insect resistance management comprises the presentation of at least one of the polypeptide of the disclosure to insects in the order Lepidoptera and/or Coleoptera.

In some embodiments, the methods of controlling Lepidoptera and/or Coleoptera insect infestation in a transgenic plant and promoting insect resistance management comprise expressing in the transgenic plant a polypeptide of the disclosure and a Cry protein or other insecticidal protein to insects in the order Lepidoptera and/or Coleoptera having different modes of action.

Also provided are methods of reducing likelihood of emergence of Lepidoptera and/or Coleoptera insect resistance to transgenic plants expressing in the plants insecticidal proteins to control the insect species, comprising expression of a polypeptide of the disclosure insecticidal to the insect species in combination with a second insecticidal protein to the insect species having different modes of action.

Also provided are means for effective Lepidoptera and/or Coleoptera insect resistance management of transgenic plants, comprising co-expressing at high levels in the plants two or more insecticidal proteins toxic to Lepidoptera and/or Coleoptera insects but each exhibiting a different mode of effectuating its killing activity, wherein the two or more insecticidal proteins comprise a polypeptide of the disclosure and a Cry protein. Also provided are means for effective Lepidoptera and/or Coleoptera insect resistance management of transgenic plants, comprising co-expressing at high levels in the plants two or more insecticidal proteins toxic to Lepidoptera and/or Coleoptera insects but each exhibiting a different mode of effectuating its killing activity, wherein the two or more insecticidal proteins comprise a polypeptide of the disclosure and a Cry protein or other insecticidally active protein.

In addition, methods are provided for obtaining regulatory approval for planting or commercialization of plants expressing proteins insecticidal to insects in the order Lepidoptera and/or Coleoptera, comprising the step of referring to, submitting or relying on insect assay binding data showing that the polypeptide of the disclosure does not compete with binding sites for Cry proteins in such insects. In addition, methods are provided for obtaining regulatory approval for planting or commercialization of plants expressing proteins insecticidal to insects in the order Lepidoptera and/or Coleoptera, comprising the step of referring to, submitting or relying on insect assay binding data showing that the polypeptide of the disclosure does not compete with binding sites for Cry proteins in such insects.

### Methods for Increasing Plant Yield

Methods for increasing plant yield are provided. The methods comprise providing a plant or plant cell expressing a polynucleotide encoding the pesticidal polypeptide sequence disclosed herein and growing the plant or a seed thereof in a field infested with a pest against which the polypeptide has pesticidal activity. In some embodiments, the polypeptide has pesticidal activity against a Lepidopteran, Coleopteran, Dipteran, Hemipteran or nematode pest, and the field is infested with a Lepidopteran, Hemipteran, Coleopteran, Dipteran or nematode pest.

As defined herein, the “yield” of the plant refers to the quality and/or quantity of biomass produced by the plant. “Biomass” as used herein refers to any measured plant product. An increase in biomass production is any improvement in the yield of the measured plant product. Increasing plant yield has several commercial applications. For example, increasing plant leaf biomass may increase the yield of leafy vegetables for human or animal consumption. Additionally, increasing leaf biomass can be used to increase production of plant-derived pharmaceutical or industrial products. An increase in yield can comprise any statistically significant increase including, but not limited to, at least a 1% increase, at least a 3% increase, at least a 5% increase, at least a 10% increase, at least a 20% increase, at least a 30%, at least a 50%, at least a 70%, at least a 100% or a greater increase in yield compared to a plant not expressing the pesticidal sequence.

In specific methods, plant yield is increased as a result of improved pest resistance of a plant expressing a polypeptide of the disclosure. Expression of the polypeptide of the disclosure results in a reduced ability of a pest to infest or feed on the plant, thus improving plant yield.

### Methods of Processing

Further provided are methods of processing a plant, plant part or seed to obtain a food or feed product from a plant, plant part or seed comprising a polynucleotide encoding a polypeptide of the disclosure. The plants, plant parts or seeds provided herein, can be processed to yield oil, protein products and/or by-products that are derivatives obtained by processing that have commercial value. Non-limiting examples include transgenic seeds comprising a nucleic acid molecule encoding a polypeptide of the disclosure which can be processed to yield soy oil, soy products and/or soy by-products.

“Processing” refers to any physical and chemical methods used to obtain any soy product and includes, but is not limited to, heat conditioning, flaking and grinding, extrusion, solvent extraction or aqueous soaking and extraction of whole or partial seeds

The following examples are offered by way of illustration and not by way of limitation.

### EXPERIMENTALS

### Example 1

### Coleopteran Assays

Bioassays with Western corn rootworm (Diabrotica virgifera virgifera LeConte, WCRW) were conducted using cell lysate samples mixed with Diabrotica diet (Frontier Agricultural Sciences, Newark, Del.). WCRW neonates were placed into each well of a 96 well plate. The assay was run four days at 25° C. and was then scored for insect mortality and stunting of larval growth. The scores were noted as dead (3), severely stunted (2) (little or no growth but alive), stunted (1) (growth to second instar but not equivalent to controls) or no activity (0). Samples demonstrating mortality or stunting were further studied.

### Example 2

### Identification of Bacterial Strains Active Against WCRW

Insecticidal activities against WCRW were observed from a clear cell lysate of bacterial strains grown in either LB medium (10 g/L tryptone, 5 g/L yeast extract, and 10 g/L NaCl) or TSB (Tryptic Soy Broth) medium (17 g/L tryptone, 3 g/L Soytone, 2.5 g/L dextrose, 2.5 g/L K₂HPO₄ and 5 g/L NaCl), 2× YT medium (yeast extract 10 g/L, pancreatic digest of casein 16 g/L, sodium chloride 5 g/L), ISP-2 medium (yeast extract, 4 g/L, malt extract, 10 g/L, dextrose, 4 g/L) and cultured as specified in Table 2. This insecticidal activity exhibited heat and proteinase sensitivity indicating proteinaceous nature. Active strains and their culture conditions are listed in Table 2.

TABLE 2

| | | Culture | Culture | | |
|---|---|---|---|---|---|
| Strain | Species | Medium | condition | Gene | Seq. No. |
| SS473A12 | Pseudomonas | TSB | 26° C., 190 rpm, | IPD092Aa-1 | SEQ ID NO: 546 |
| | rhodesiae | | 2 days | IPD092-2Aa | SEQ ID NO: 547 |
| SS232H12 | Serratia | TSB | 26° C., 190 rpm, | IPD095Aa-1 | SEQ ID NO: 562 |
| | nematophilia | | 2 days | IPD095-2Aa | SEQ ID NO: 563 |
| JH58776-1 | Haemophilus | 2xYT | 28° C., 200 rpm, | IPD097Aa | SEQ ID NO: 590 |
| | piscium | | 1 day | IPD099Aa-1 | SEQ ID NO: 591 |
| | | | | IPD099-2Aa | SEQ ID NO: 592 |
| | | | | IPD099Aa-3 | SEQ ID NO: 593 |
| JH55673-1 | Pseudomonas | TSB | 28° C., 160 rpm, | IPD100Aa-1 | SEQ ID NO: 611 |
| | gessardii | | 1 day | IPD100-2Aa | SEQ ID NO: 612 |
| JH90961-1 | Chromobacterium | 2xYT | 28° C., 200 rpm, | IPD105Aa | SEQ ID NO: 614 |
| | aquaticum | | 1 day | | |
| JH48820-1 | Chitinophaga | 2xYT | 28° C., 200 rpm, | IPD106Aa-1 | SEQ ID NO: 617 |
| | pinensis | | 1 day | IPD106-2Aa | SEQ ID NO: 618 |
| JH60888-1 | Pseudomonas | ISP-2 | 26° C., 250 rpm, | IPD107Aa | SEQ ID NO: 621 |
| | brassicacearum | | 1 day | | |
| JH59138-1 | Burkholderia | 2xYT | 28° C., 160 rpm, | IPD111Aa | SEQ ID NO: 629 |
| | ambifaria | | 1 day | | |
| SSP640H4-1 | Pseudomonas | TSB | 26° C., 210 rpm, | IPD112Aa | SEQ ID NO: 635 |
| | vranovensis | | 2 days | | |

### Example 3

### Species Identification and Genome SequencinG of Active Strains

Genomic DNA from active strains was extracted with a Sigma® Bacterial Genomic DNA Extraction Kit (Cat #NA2110-KT, Sigma-Aldrich, PO Box 14508, St. Louis, Mo. 63178) according to the manufactures' instructions. The DNA concentration was determined using a NanoDrop™ Spectrophotometer (Thermo Scientific, Wilmington, Del.) and the genomic DNA was diluted to 40 ng/μL with sterile water. A 25 μL PCR reaction was set up by combining 80 ng genomic DNA, 2 μL (5 μM) 16S ribosomal DNA primers TACCTTGTTACGACTT (SEQ ID NO: 639) and AGAGTTTGATCMTGGCTCAG (SEQ ID NO: 640), 1 μL 10 cmM dNTP, 1× Phusion® HF buffer, and 1 unit of Phusion® High-Fidelity DNA Polymerase (New England Biolabs, Ipswich, Mass.). The PCR reaction was run in MJ Research PTC-200 Thermo Cycler (Bio-Rad Laboratories, Inc., Hercules, Calif.) with the following program: 96° C. 1 min; 30 cycles of 96° C. 15 seconds, 52° C. 2 minutes and 72° C. 2 minutes; 72° C. 10 minutes; and hold on 4° C. The PCR products were purified with QiaQuick® DNA purification Kit (Cat #28104, QIAGEN Inc., Valencia, Calif.). The purified PCR sample was DNA sequenced and the resulting 16S ribosomal DNA sequence was BLAST® searched against the NCBI database for indication of the species of the strain (see Table 2).

Genomic DNA of active strains was also prepared according to a library construction protocol developed by Illumina (San Diego, Calif.) and sequenced using the Illumina MiSeq™. The nucleic acid contig sequences were assembled and open reading frames were generated.

### Example 4

### Identification of Insecticidal Proteins by LC-MS/MS

All insecticidal proteins were fractionated and enriched as described. For identification candidate protein bands from SDS PAGE gels were excised, digested with trypsin and analyzed by nano-liquid chromatography/electrospray tandem mass spectrometry (nano-LC/ESI-MS/MS) on a Thermo Q Exactive™ Orbitrap™ mass spectrometer (Thermo Fisher Scientific®) interfaced with an Eksigent® NanoLC Ultra 1D Plus™ NanoLC system (AB Sciex). Alternatively, the proteins in the chromatography fractions were directly digested with trypsin and then analyzed by nano-LC/ESI-MS/MS. Ten product ion spectra were collected in data dependent acquisition mode after a precursor ion survey scan.

Protein identification was done by database searches using Mascot® (Matrix Science). The databases were an in-house database “Bacteria-purify” which contains annotated protein sequences of bacterial genomes, as well as other in-house protein sequence databases, and Swiss-Prot.

### Example 5

### Isolation and Identification of Insecticidal Proteins

### Isolation and Identification of IPD092-1Aa and IPD092-2Aa

Insecticidal activity against WCRW (Diabrotica virgifera virgifera) was observed from crude cell lysates of Pseudomonas rhodesiae Strain SS473A12 grown in TSB (Tryptic Soy Broth) medium (17 g/L tryptone, 3 g/L Soytone, 2.5 g/L dextrose, 2.5 g/L K₂HPO₄ and 5 g/L NaCl), at 26° C., with shaking at 190 rpm for 2 days. This insecticidal activity exhibited heat and protease sensitivity indicating a proteinaceous nature.

Cell pellets of strain SS473A12 were lysed at ˜30,000 psi (Constant Systems Ltd. Low March, Daventry Northants, United Kingdom) after re-suspension in 25 mM MES buffer, pH 6 with Protease Inhibitor Cocktail Set V, EDTA-Free (Calbiochem/EMD Millipore, Darmstadt,

Germany). The crude lysate was cleared by centrifugation, readjusted to pH 6 and loaded onto a tandem of two 5 mL HiTrap™ SP-HPTM (GE Healthcare, Piscataway, N.J.) columns equilibrated in MES buffer, pH 6. Insecticidal activity eluted with a gradient of 0 to 300 mM NaCl over 20 column volumes (CV). Active fractions were pooled, adjusted to 1 M ammonium sulfate by addition of a 4 M ammonium sulfate stock solution, and loaded onto a tandem of two 1 mL HiTrap™ Phenyl HPTM (GE Healthcare, Piscataway, N.J.) columns that were equilibrated in 25 mM MES pH 6. A gradient from 1 to 0 M ammonium sulfate was applied over 22 CV. Eluted active fractions were pooled and buffer exchanged using Zeba™ (Thermo Scientific) columns to 25 mM Tris, pH 8, and loaded onto a Mono Q™ (GE Healthcare, Piscataway, N.J.) column equilibrated in 25 mM Tris, pH 8. Active fractions eluted with a gradient from 0 to 500 mM NaCl over 30 CV. Active fractions were pooled, concentrated on 10 kDa molecular weight cutoff centrifugal concentrators (Sartorius Stedim, Goettingen, Germany) and loaded onto a Superdex™ 200 column (GE Healthcare) equilibrated in 25 mM Tris pH 8, 150 mM NaCl. SDS-PAGE analysis of fractions indicated that WCRW activity coincided with two prominent bands after staining with InstantBlue™ (Expedeon Ltd., San Diego, Calif.). The approximately 21 and 22 kDa protein bands were excised, digested with trypsin and analyzed by nano-liquid chromatography/electrospray tandem mass spectrometry (nano-LC/ESI-MS/MS) as described in Example 4. Searches against an in-house database, including the genomic sequence of SS473A12 which was generated as described in Example 3 identified the IPD092-1Aa polypeptide (SEQ ID NO: 1) and IPD092-2Aa (SEQ ID NO: 2) which are encoded by the polynucleotides of SEQ ID NO: 546 and SEQ ID NO: 547, respectively. The genes encoding IPD092-1Aa and IPD092-2Aa were in a single operon.

Cloning and recombinant co-expression confirmed the insecticidal activity of the IPD092-1Aa polypeptide (SEQ ID NO: 1) and IPD092-2Aa polypeptide (SEQ ID NO: 2) against WCRW.

### Isolation and Identification of IPD095-1Aa and IPD095-2Aa

Insecticidal activity against WCRW (Diabrotica virgifera virgifera) was observed from cleared cell lysates of Serratia nematophilia Strain SS232H12 grown in TSB (Tryptic Soy Broth) medium (17 g/L tryptone, 3 g/L Soytone, 2.5 g/L dextrose, 2.5 g/L K₂HPO₄ and 5 g/L NaCl), at 26° C., with shaking at 190 rpm for 2 days. This insecticidal activity exhibited heat and protease sensitivity indicating a proteinaceous nature.

Cell pellets of strain SS473A12 were lysed at ˜30,000 psi (Constant Systems Ltd. Low March, Daventry Northants, United Kingdom) after re-suspension in 25 mM acetate, pH 5, with Protease Inhibitor Cocktail Set V, EDTA-Free (Calbiochem/EMD Millipore, Darmstadt, Germany). The crude lysate was cleared by centrifugation, readjusted to pH 5 and loaded onto a tandem of two 1 mL HiTrap™ SP-HP (GE Healthcare, Piscataway, N.J.) columns equilibrated in 25 mM acetate, pH 5. Bound proteins were eluted with a gradient of 0 to 350 mM NaCl over 20 column volumes (CV). Individual eluting fractions were inactive against WCRW but a combination of the weakly active unbound proteins with fractions eluting between a conductivity of 19 to 26 mSi resulted in strongly active pools suggesting that a two-component protein is responsible for WCRW activity. The eluted fraction pool from the HiTrap™ SP-HP columns containing active components was concentrated on 10 kDa molecular weight cutoff centrifugal concentrators (Sartorius Stedim, Goettingen, Germany) and then loaded onto a Superdex™ 75 (GE Healthcare) column that was equilibrated in 25 mM Na-Acetate, pH 5, 100 mM NaCl. Eluted fractions were inactive when assayed directly but were insecticidal when combined with the proteins that were not bound in the previous step by the SP columns. SDS-PAGE analysis of fractions indicated that WCRW activity coincided with two bands after staining with InstantBlue™ (Expedeon Ltd., San Diego, Calif.). The approximately 19 and 60 kDa protein bands were excised, digested with trypsin and analyzed by nano-liquid chromatography/electrospray tandem mass spectrometry (nano-LC/ESI-MS/MS) as described in Example 1. Protein identification was done by searches in various databases, including the genomic sequence of SS232H12 which was generated as described in Example 3. This identified the IPD095-1Aa polypeptide (SEQ ID NO: 27) and IPD095-2Aa polypeptide (SEQ ID NO: 28) which are encoded by the polynucleotides of SEQ ID NO: 562 and SEQ ID NO: 563, respectively. The genes encoding IPD095-1Aa and IPD095-2Aa were in a single operon. Recombinant co-expression of IPD095-1Aa (SEQ ID NO: 562) and IPD095-2Aa (SEQ ID NO: 563) in E. coli confirmed insecticidal activity against WCRW of the polypeptides IPD095-1Aa (SEQ ID NO: 27) and IPD095-2Aa (SEQ ID NO: 28). At the concentrations tested neither IPD095-1Aa (SEQ ID NO: 27) nor IPD095-2Aa (SEQ ID NO: 28) alone showed insecticidal activity against WCRW.

### Isolation and Identification of IPD097Aa and IPD099-1Aa, IPD099-2Aa, and IPD099-3Aa

Insecticidal activity against WCRW (Diabrotica virgifera virgifera) was observed from cleared cell lysates of Haemophilus piscium Strain JH58776-1 grown in 2× YT (yeast extract 10 g/L, pancreatic digest of casein 16 g/L, sodium chloride 5 g/L) at 28° C. while shaking at 200 rpm for 1 day. This insecticidal activity exhibited heat and protease sensitivity indicating a proteinaceous nature.

Cell pellets of strain JH58776-1 were lysed at ˜30,000 psi (Constant Systems Ltd. Low March, Daventry Northants, United Kingdom) after re-suspension in 25 mM Tris, pH 8, with “Complete, EDTA-free” protease inhibitor cocktail (Roche, Indianapolis, Ind.). The crude lysate was cleared by centrifugation at 30,000 g for 40 min and then brought to 30% ammonium sulfate saturation by slow addition of 100% saturated ammonium sulfate. After stirring for 1 hr., the solution was centrifuged at 30,000 g for 15 min. The supernatant was then brought to 70% saturation by addition of 100% saturated ammonium sulfate. This solution was stirred for 1 hr. and then centrifuged at 25,000 g for 15 min. The pellet was suspended in 20 mM Tris pH 8.0 and then adjusted to ˜1 M ammonium sulfate by addition of 2 M ammonium sulfate, 20 mM Tris, pH 8.0. The suspended extract was centrifuged at 30,000 g for 30 min and the supernatant loaded onto a 20 mL Phenyl-5PW (Tosoh Bioscience, South San Francisco, Calif.) column that was equilibrated in 20 mM Tris, pH 8.0 with 1 M ammonium sulfate. After elution of unbound proteins, a 5 CV linear gradient to 20 mM Tris, pH 8.0, 0% ammonium sulfate was applied. Eluted WCRW active fractions were pooled and concentrated using 10 kDa molecular weight cutoff centrifugal concentrators (Sartorius Stedim, Goettingen, Germany). The concentrated pool was desalted using a 26/10 G25 column (GE Healthcare) equilibrated in 20 mM Tris, pH 8.0 and then loaded onto an 8 mL GigaCap™ Q column (Tosoh Bioscience, King of Prussia, Pa.) equilibrated 20 mM Tris, pH 8.0. The column was washed with 3 CV, and then a 7.5 CV gradient to 20 mM Tris, pH 8, 0.4 M NaCl was applied. Eluted WCRW active fractions were pooled, concentrated and desalted using a 5 mL Hi-Trap™ (GE Healthcare) desalting column equilibrated in 25 mM Bis-Tris/iminodiacetic acid, pH 7.0. The desalted sample was loaded onto a 4 mL Mono P™ chromatofocusing column (GE Healthcare) equilibrated in 25 mM Bis-Tris/iminodiacetic acid, pH 7.0 and washed with 2 CV. The buffer was then switched to 1/10× Polybuffer 7-4 (GE Healthcare)/iminodiacetic acid, pH 4.0. Eluate fractions were assayed for WCRW activity and subjected to SDS PAGE analysis and staining with GelCode Blue® stain reagent (Thermo Scientific, Rockford, Ill.).

Two major zones of WCRW activity eluted from the chromatofocusing column. The first zone of WCRW activity eluted early in the pH gradient with fractions D5 and D6 coinciding with a single stained band on a SDS-PAGE gel. The approximately 16 kDa protein band was excised, digested with trypsin and analyzed by nano-LC/ESI-MS/MS as described in Example 1. Searches were conducted against various databases including the genomic sequence of JH58776-1. This identified the IPD097Aa polypeptide (SEQ ID NO: 121) which is encoded by the polynucleotides of SEQ ID NO: 590. Recombinant expression of IPD097Aa (SEQ ID NO: 590) in E. coli confirmed insecticidal activity of the polypeptide IPD097Aa (SEQ ID NO: 121). The second zone of WCRW activity eluted later in the pH gradient with fractions E5 to E7 coinciding with a band on a SDS-PAGE gel after staining with Blue® stain reagent (Thermo Fisher Scientific®). The approximately 38 kDa protein band was excised, digested with trypsin and analyzed by nano-LC/ESI-MS/MS as described in Example 1. Searches were conducted against various databases including the genomic sequence of JH58776-1. Edman sequencing allowed for identification of the N-terminal of this protein. This identified the IPD099-2Aa polypeptide (SEQ ID NO: 137) which is encoded by the polynucleotide of SEQ ID NO: 592. Recombinant expression of IPD099-2Aa (SEQ ID NO: 592) in E. coli confirmed the insecticidal activity against WCRW of the IPD099-2Aa polypeptide (SEQ ID NO: 137). Analysis of the genomic sequence of JH58776-1 indicated that the IPD099-2Aa gene was part of an operon that encodes two additional proteins, IPD099-1Aa (SEQ ID NO: 136) encoded by the polynucleotides of SEQ ID NO: 591 and IPD099-3Aa (SEQ ID NO: 138) encoded by the polynucleotides of SEQ ID NO: 593. Analysis of eluted chromatofocusing column fractions by nano-LC/ESI-MS/MS indicated that the IPD099-1Aa (SEQ ID NO: 136) and IPD099-3Aa (SEQ ID NO: 138) polypeptides eluted in fractions F10 to F1. A pool of fractions containing the IPD099-1Aa polypeptide (SEQ ID NO: 136), IPD099-2Aa polypeptide (SEQ ID NO: 137) and IPD099-3Aa polypeptide (SEQ ID NO: 138) was active at a concentration where the IPD099-2Aa polypeptide (SEQ ID NO: 137) alone was inactive. Based on these sequences PCR primers were designed and used to amplify the IPD099-1/2/3Aa operon as well as the individual IPD099-1Aa (SEQ ID NO: 136), IPD099-2Aa (SEQ ID NO: 137) and IPD099-3Aa (SEQ ID NO: 138) genes from genomic DNA prepared from the JH58776-1 bacterial cells.

The IPD099-1/2/3Aa operon as well as the individual component genes were cloned for expression of N-terminally 6× His tagged using pET14a or untagged proteins using pET-24a (No tag) via seamless cloning and transformed into BL21-Gold E. coli cells. The untagged IPD099-1/2/3Aa operon construct resulted in soluble expression of the three proteins and activity against WCRW when added to artificial diet. The cleared lysate of cells expressing untagged IPD099-2Aa polypeptide (SEQ ID NO: 137) alone resulted in severe stunting of WCRW when added to diet at 45 μg/cm2. A mixture of cleared lysates of cells expressing the IPD099-1Aa polypeptide (SEQ ID NO: 136), IPD099-2Aa polypeptide (SEQ ID NO: 137) and IPD099-3Aa polypeptide (SEQ ID NO: 138), each individually as untagged protein resulted in strong activity against WCRW. Table 3 shows average WCRW activity scores (n=4) resulting from assaying various combinations of purified N-6× His tagged IPD099-2Aa polypeptide (SEQ ID NO: 137) with purified tagged IPD099-1Aa polypeptide (SEQ ID NO: 136) and IPD099-3Aa polypeptide (SEQ ID NO: 138). While the IPD099-2Aa polypeptide (SEQ ID NO: 137) alone lost activity after 2 or more-fold dilution of a dose of 30 μg/cm2, stunting of WCRW was observed with an assay mixture containing 3.8, 1.9, and 4.7 μg/cm2 of the IPD099-1Aa polypeptide (SEQ ID NO: 136), IPD099-2Aa polypeptide (SEQ ID NO: 137), and IPD099-3Aa polypeptide (SEQ ID NO: 138), respectively. Furthermore, adding 75 pg/cm2 of the IPD099-3Aa polypeptide (SEQ ID NO: 138) to 60 μg/cm2 of the IPD099-1Aa polypeptide (SEQ ID NO: 136) resulted in no activity against WCRW and adding 75 μg/cm2 of the IPD099-3Aa polypeptide (SEQ ID NO: 138) to 30 μg/cm2 of the IPD099-2Aa polypeptide (SEQ ID NO: 137) didn't result in increased activity against WCRW compared to IPD099-2Aa (SEQ ID NO: 137) alone. A mixture of 60 μg/cm2 of the IPD099-1Aa polypeptide (SEQ ID NO: 136) with 30 ∞g/cm2 of the IPD099-2Aa polypeptide (SEQ ID NO: 137) resulted in severe stunting and stunting could still be observed with a 2-fold diluted mixture of these.

TABLE 3

| | Dose of N-6xHis-tagged IPD099 component in | | | | | | | | |
|---|---|---|---|---|---|---|---|---|---|
| | assay mixture (μg/cm²) | | | | | | | | |
| | IPD099-1Aa | IPD099-2Aa | IPD099-3Aa | | | | | | |
| | SEQ ID NO: | SEQ ID NO: | SEQ ID NO: | Fold-dilution of assay mixture | | | | | |
| Sample ID | 136 | 137 | 138 | 1 | 2 | 4 | 8 | 16 | 32 |
| IPD099-2 | — | 30 | — | 1 | 0.5 | 0 | 0 | 0 | 0 |
| IPD099-1-2 | 60 | 30 | — | 2 | 1.25 | 0 | 0 | 0 | 0 |
| IPD099-2-3 | — | 30 | 75 | 0.25 | 0 | 0 | 0 | 0 | 0 |
| IPD099-1-3 | 60 | — | 75 | 0 | 0 | 0 | 0 | 0 | 0 |
| IPD099-1-2-3 | 60 | 30 | 75 | 3 | 2.75 | 2.5 | 1.5 | 1 | 0 |

### Isolation and Identification of IPD100Aa-1/2

Insecticidal activity against WCRW (Diabrotica virgifera virgifera) was observed from cleared cell lysates of strain JH55673-1 (Pseudomonas gessardii) grown in TSB (Tryptic Soy Broth) medium (17 g/L tryptone, 3 g/L Soytone, 2.5 g/L dextrose, 2.5 g/L K₂HPO₄ and 5 g/L NaCl), at 28° C., while shaking at 160 RPM, 24 hours. This insecticidal activity exhibited heat and protease sensitivity indicating a proteinaceous nature.

Cell pellets of strain JH55673-1 were suspended in 20 mM Tris, pH 8.0+1:100 Halt™ proteinase inhibitor cocktail and lysed at 30,000 psi (Constant Systems Ltd. Low March, Daventry Northants, United Kingdom). Lysed extract was then centrifuged at 30,000 g for 45 min. The supernatant was brought to 60% ammonium sulfate saturation by dropwise addition of 100% saturated ammonium sulfate while stirring in a cold room overnight. The extract was centrifuged at 30,000 g for 20 minutes and the supernatant was discarded. The pellet portion was resuspended in 20 mM Tris, pH 8.0 and then adjusted to 1 M ammonium sulfate by dropwise addition of 2 M ammonium sulfate, 20 mM Tris, pH 8.0. After clarification, the supernatant was loaded onto a 20 mL TSKgel ether-5PW column (Tosoh Bioscience) equilibrated in 20 mM Tris, pH 8.0, 1 M ammonium sulfate. The column was washed with 3 CV, and then a 7.5 CV gradient to 20 mM Tris, pH 8.0 was applied. Eluted WCRW-active fractions were pooled and concentrated using 5 kDa centrifugal concentrators (Sartorius Stedim, Goettingen, Germany) and then desalted into 20 mM Tris, pH 8.0 using a 26/10 G25 desalting column (GE Healthcare). The desalted fraction pool was loaded onto an 8 mL SuperQ™-5PW column (Tosoh Bioscience, King of Prussia, Pa.). The column was washed with 4 CV, and then a 20 CV gradient was started to 20 mM Tris, pH 8, 0.3 M NaCl. WCRW-active fractions from the anion exchange column were pooled, concentrated and loaded onto a tandem set of two 10×300 mm Superdex™ 75 (GE Healthcare) size exclusion chromatography columns equilibrated in PBS buffer. SDS-PAGE analysis indicated that eluted WCRW activity coincided with two bands after staining with GelCode Blue® stain reagent (Thermo Fisher Scientific®). The approximately 17 and 57 kDa protein bands were excised, digested with trypsin and analyzed by nano-liquid chromatography/electrospray tandem mass spectrometry (nano-LC/ESI-MS/MS) as described in Example 1. Protein identification was done by searches in various databases, including the genomic sequence of JH55673-1 which was generated as described in Example 3. This identified the IPD100-1Aa polypeptide (SEQ ID NO: 332) and IPD100-2Aa polypeptide (SEQ ID NO: 333) which are encoded by the polynucleotides of SEQ ID NO: 611 and SEQ ID NO: 612, respectively. The genes encoding IPD100-1Aa (SEQ ID NO: 611) and IPD100-2Aa (SEQ ID NO: 612 were in a single operon. Recombinant co-expression of IPD100-1Aa (SEQ ID NO: 611) and IPD100-2Aa (SEQ ID NO: 612) in E. coli confirmed insecticidal activity of the polypeptides IPD100-1Aa (SEQ ID NO: 332) and IPD100-2Aa (SEQ ID NO: 333). At the concentrations tested neither IPD100-1Aa (SEQ ID NO: 611) nor IPD100-2Aa (SEQ ID NO: 612) alone showed insecticidal activity against WCRW.

### Isolation and Identification of IPD105Aa

Insecticidal activity against WCRW (Diabrotica virgifera virgifera) was observed from cleared cell lysates of strain JH90961-1 (Chromobacterium aquaticum) grown in 2× YT medium (yeast extract 10 g/L, pancreatic digest of casein 16 g/L, sodium chloride 5 g/L), at 28° C., while shaking at 200 RPM for 1 day. This insecticidal activity exhibited heat and protease sensitivity indicating a proteinaceous nature.

Cell pellets of strain JH90961-1 (Chromobacterium aquaticum) were suspended in PBS, centrifuged at 30,000 g for 30 min. The supernatant was discarded, the cell pellet frozen and then thawed before resuspending in 20 mM Tris, pH 9 plus “Complete, EDTA-free” protease inhibitor cocktail (Roche, Indianapolis, Ind.), and lysed at 30,000 psi (Constant Systems Ltd. Low March, Daventry Northants, United Kingdom). Lysed extract was then centrifuged at 30,000 g for 30 min. The supernatant was filtered and then diluted 1:1 with 20 mM Tris, pH 9 and loaded onto a 10 mL Capto™ Q column (GE Healthcare) equilibrated in 20 mM Tris, pH 9. After elution of unbound proteins 20 mM Tris, 0.6 M NaCl, pH 9 was used to elute WCRW-active proteins. An aliquot of the Capto™ Q eluate was desalted into 25 mM BisTris, pH 7.4 and loaded onto a 4 mL Mono P™ (GE Healthcare) chromatofocusing column with a 100% B isocratic gradient (Buffer B: Polybuffer 74, pH 4.4—diluted 1:10 with H₂O. WCRW-active fractions were pooled and desalted into 20 mM Tris, pH 8 and loaded onto a 1 mL Mono Q™ (GE Healthcare) anion exchange column over a 30 CV gradient to 20 mM Tris+0.5 M NaCl, pH 8). WCRW-activity was observed with fractions eluting at a conductivity of 9.9-14.9 mS/cm. SDS-PAGE analysis indicated that eluted WCRW activity coincided with a band after staining with GelCode Blue® stain reagent (Thermo Fisher Scientific®). The approximately 19 kDa protein band were excised, digested with trypsin and analyzed by nano-liquid chromatography/electrospray tandem mass spectrometry (nano-LC/ESI-MS/MS) as described in Example 1. Protein identification was done by searches in various databases, including the genomic sequence of JH90961-1 which was generated as described in Example 3. This identified the IPD105Aa polypeptide (SEQ ID NO: 350) which is encoded by the polynucleotide of SEQ ID NO: 614. Recombinant expression of IPD105Aa (SEQ ID NO: 614) in E. coli confirmed insecticidal activity of the polypeptide IPD105Aa (SEQ ID NO: 350).

### Isolation and Identification of IPD106Aa-1/2

Insecticidal activity against WCRW (Diabrotica virgifera virgifera) was observed from cleared cell lysates of strain JH48820-1 (Chitinophaga pinensis) grown 2× YT medium (yeast extract 10 g/L, pancreatic digest of casein 16 g/L, sodium chloride 5 g/L), at 28° C., while shaking at 200 RPM for 1 day. This insecticidal activity exhibited heat and protease sensitivity indicating a proteinaceous nature.

Cell pellets of strain JH48820-1 (Chitinophaga pinensis) were suspended in PBS, centrifuged at 30,000 g for 30 min. The supernatant was discarded and the cell pellet frozen and then thawed before resuspending in 20 mM Tris, pH 8 plus “Complete, EDTA-free” protease inhibitor cocktail (Roche, Indianapolis, Ind.), and lysed at 30,000 psi (Constant Systems Ltd. Low March, Daventry Northants, United Kingdom). Lysed extract was then centrifuged at 30,000 g for 30 min. The supernatant was filtered and then 0.5 M Na-Formate, pH 4 was added (1:10) to a final conc. of 50 mM, and 1% formic acid added to reduce the pH to pH 4. This was rocked in a cold room and clarified by centrifugation before diluting the supernatant 1:1 with 50 mM Na-Formate, pH 4 and loading this onto a 1 mL CaptoTMS (GE Healthcare) cation exchange chromatography column. After elution of unbound proteins 50 mM Na-Formate, pH 4 with 0.3 M NaCl was used to elute WCRW-active proteins. The Capto™S eluate was then concentrated with 10 kDa MWCO centrifugal concentrators (Sartorius Stedim, Goettingen, Germany) before loading onto a tandem of two Superdex™ 200 (GE Healthcare) size exclusion chromatography columns equilibrated in 100 mM ammonium bicarbonate. WCRW active fractions were pooled and desalted into 20 mM Tris, pH 8.7 and loaded onto a 1 mL Mono QTM (GE Healthcare) anion exchange chromatography column and a 30 CV gradient to 20 mM Tris+0.35 M NaCl, pH 8.7 was applied. Severe WCRW stunting activity was observed in fractions eluting at a conductivity of 5.6-8.5 mS/cm. SDS-PAGE analysis indicated that eluted WCRW activity coincided with two bands after staining with GelCode Blue® stain reagent (Thermo Fisher Scientific®). The approximately 76 and 45 kDa protein bands were excised, digested with trypsin and analyzed by nano-liquid chromatography/electrospray tandem mass spectrometry (nano-LC/ESI-MS/MS) as described in Example 1. Protein identification was done by searches in various databases, including the genomic sequence of JH48820-1 which was generated as described in Example 3. This identified the IPD106-1Aa polypeptide (SEQ ID NO: 366) and IPD106-2Aa polypeptide (SEQ ID NO: 367) which are encoded by the polynucleotides of SEQ ID NO: 617 and SEQ ID NO: 618, respectively. The genes encoding IPD106-1Aa and IPD106-2Aa were in a single operon. Recombinant expression of IPD106-1Aa (SEQ ID NO: 617) and IPD106-2Aa (SEQ ID NO: 618) in E. coli confirmed insecticidal activity of the polypeptides IPD1061Aa (SEQ ID NO: 366) and IPD106-2Aa (SEQ ID NO: 367). At the concentrations tested neither the IPD100-1Aa (SEQ ID NO: 366) nor IPD100-2Aa (SEQ ID NO: 367) polypeptides alone showed insecticidal activity against WCRW.

### Isolation and Identification of IPD107Aa

JH60888-1 (Pseudomonas brassicacearum) was grown in ISP-2 Medium (Yeast Extract—4 g/L, Malt Extract—10 g/L, Dextrose—4 g/L) at 26° C. while shaking at 250 rpm for 1 day. This insecticidal activity exhibited heat and protease sensitivity indicating a proteinaceous nature.

Cell pellets of JH60888-1 were suspended in B-PER II Bacterial Protein Extraction Reagent (Thermo Pierce) diluted to ¼× strength in 20 mM Tris-HCl buffer, pH 9.0 (Buffer A) containing protease inhibitor cocktail V from CalBiochem, Ready-Lyse™ lysozyme from Epicentre and OmniCleave™ endonuclease from Epicentre (Madison, Wis.). The cell suspension was incubated at 30° C., 250 rpm for 1 hour. The crude lysate was cleared by centrifugation at 20,000 g for 10 min and adjusted to pH 8.7 with 1 N NaOH. This material was loaded onto an anion exchange column packed with Q Sepharose™ HP media (GE Healthcare) equilibrated in Buffer A. Bound protein was eluted with a linear gradient to 0.5 M NaCl in Buffer A. Fractions were desalted and subjected for identification of insecticidal activity. Active fractions were pooled, buffer exchanged into 1M ammonium sulfate, 20 mM Tris-HCl, pH 9 (Buffer B) and applied to a hydrophobic interaction Phenyl Sepharose™ HP column (GE Healthcare) equilibrated in Buffer B. Protein was eluted with a linear gradient from 1 M to 0 M ammonium sulfate. Fractions were desalted and subjected for identification of insecticidal activity. Active fractions were pooled, desalted into 20 mM Tris-HCl pH 8, 150 mM NaCl (Buffer C) and concentrated to a final volume of 0.4 mL in a 10,000 MWCO membrane (GE Healthcare). The concentrated material was loaded onto a Superdex™ 200 10/30 size exclusion column (GE Healthcare) in Buffer C. SDS-PAGE analysis of fractions with WCRW activity showed a dominant band after staining with Coomassie® Blue dye. The approximately 11 kDa protein band was excised, digested with trypsin and analyzed by nano-liquid chromatography/electrospray tandem mass spectrometry (nano-LC/ESI-MS/MS) as described in Example 1 and subjected to N-terminal amino acid sequencing by Edman degradation. Protein identification was done by searches in various databases, including the genomic sequence of JH60888-1 which was generated as described in Example 3. This identified the IPD107Aa polypeptide (SEQ ID NO: 377) which is encoded by the polynucleotide of SEQ ID NO: 621. Recombinant expression of IPD107Aa (SEQ ID NO: 621) in E. coli confirmed insecticidal activity of the IPD107Aa polypeptide (SEQ ID NO: 377).

### Isolation and Identification of IPD111Aa

JH59138-1 (Burkholderia ambifaria) were grown in n 2× YT medium (yeast extract 10 g/L, pancreatic digest of casein 16 g/L, sodium chloride 5 g/L) at 28° C. while shaking at 160 rpm for 1 day. This insecticidal activity exhibited heat and protease sensitivity indicating a proteinaceous nature.

Cell pellets of strain JH59138-1 (Burkholderia ambifaria) were suspended in 20 mM MOPS, pH 8 buffer with “Complete, EDTA-free” protease inhibitor cocktail (Roche, Indianapolis, Ind.) and lysed at 30,000 psi (Constant Systems Ltd. Low March, Daventry Northants, United Kingdom). The crude lysate was cleared by centrifugation and filtration and adjusted to 1.0 M ammonium sulfate. The cleared lysate was loaded onto a HiTrap™ PhenylHP column (GE Healthcare, Piscataway, N.J.) equilibrated in 20 mM MOPS, pH 7.0, 1.0 M ammonium sulfate and eluted with a gradient to 0% ammonium sulfate in 20 mM MOPS, pH 7.0. Active fractions were pooled and desalted into 20 mM Tris, pH 8.0 using a HiPrep™ 26/10 desalting column (GE Healthcare) then loaded onto a Q-Sepharose™ FF column (GE Healthcare) equilibrated in 20 mM Tris, pH 8.0 and eluted with a gradient of 0 to 0.4 M NaCl over 30 column volumes. Active fractions were pooled and desalted into 25 mM Bis-Tris, pH 6.6 using a HiPrep™ 26/10 desalting column (GE Healthcare) then loaded onto a Mono P™ column (GE Healthcare) equilibrated in 25 mM Bis-Tris, pH 6.6 and eluted with 100% Polybuffer 74, pH 4.0 over 15 column volumes. Active fractions were pooled and desalted into 20 mM MES, pH 6.0 using a HiPrep™ 26/10 desalting column (GE Healthcare) then loaded onto a Mono Q™ column (GE Healthcare) equilibrated in 20 mM MES, pH 6.0 and eluted with a gradient of 0 to 0.2 M NaCl over 40 column volumes. SDS-PAGE analysis of fractions indicated that WCRW activity coincided with a prominent band after staining with GelCode™ Blue Stain Reagent (Thermo Fisher Scientific®). The approximately 36 kDa protein band was excised, digested with trypsin and analyzed by nano-liquid chromatography/electrospray tandem mass spectrometry (nano-LC/ESI-MS/MS) as described in Example 1. Protein identification was done by searches in various databases, including the genomic sequence of JH59138-1 which was generated as described in Example 3. This identified the IPD111Aa polypeptide (SEQ ID NO: 453) which is encoded by the polynucleotide of SEQ ID NO: 629. Recombinant expression of IPD111Aa (SEQ ID NO: 629) in E. coli confirmed insecticidal activity of the IPD111Aa polypeptide (SEQ ID NO: 453).

### Isolation and Identification of IPD112Aa

SSP 640H4-1 (Burkholderia ambifaria) was grown in TSB (Tryptic Soy Broth) medium (17 g/L tryptone, 3 g/L Soytone, 2.5 g/L dextrose, 2.5 g/L K₂HPO₄ and 5 g/L NaCl), at 26° C. while shaking at 210 rpm for 2 days. This insecticidal activity exhibited heat and protease sensitivity indicating a proteinaceous nature.

Cell pellets of strain SSP 640H4-1 (Burkholderia ambifaria) were suspended in 30 mM MES, pH 6 buffer, EMD Millipore protease inhibitor cocktail V (Merck KGaA, Darmstadt, Germany) at 1:100 volume, OmniCleave™ Endonuclease and ReadyLyse lysozyme (Epicenter Technologies Corporation, Chicago, Ill., USA) with “Complete, EDTA-free” protease inhibitor cocktail (Roche, Indianapolis, Ind.) and lysed at 30,000 psi (Constant Systems Ltd. Low March, Daventry Northants, United Kingdom). The crude lysate was cleared by centrifugation and filtration and brought to pH 6 by addition of 1.0 N HCl. The lysate was clarified by centrifugation at 13,800 g at 4° C. for 15 min. and then loaded onto a cation-exchange HiTrap™ S FF (GE Healthcare) column that was equilibrated in 30 mM MES, pH 6. WCRW active fractions eluted with a 30-column volume gradient to 30 mM MES pH 6, 0.6 M NaCl. Active fractions were pooled and concentrated using VivaSpin™ centrifugal concentrator with a 10 kDa molecular weight cut-off (Sartorius Stedim, Goettingen, Germany) and clarified by centrifuging at 10,000 g for 15 min. The concentrated and clarified fraction pool was then loaded onto a Superdex™ Increase 200 10/300 GL size exclusion column (GE Healthcare, Piscataway, N.J.) equilibrated in 30 mM MES with 0.15 M NaCl. SDS-PAGE analysis of fractions indicated that WCRW activity coincided with a prominent band after staining with GelCode™ Blue Stain Reagent (Thermo Fisher Scientific®). The approximately 33 kDa protein band was excised, digested with trypsin and analyzed by nano-liquid chromatography/electrospray tandem mass spectrometry (nano-LC/ESI-MS/MS) as described in Example 1. Protein identification was done by searches in various databases, including the genomic sequence of SSP 640H4-1 which was generated as described in Example 3. This identified the IPD112Aa polypeptide (SEQ ID NO: 529) which is encoded by the polynucleotide of SEQ ID NO: 635. Recombinant expression of IPD112Aa (SEQ ID NO: 635) in E. coli confirmed insecticidal activity of the IPD112Aa polypeptide (SEQ ID NO: 529).

### Example 6

### Gene Cloning and E. coil Expression

The target genes encoding the insecticidal proteins were first amplified by PCR using their genomic DNA as templates. The PCR primers were designed based on the 5′ end and 3′ end sequences of the gene either with appropriate restriction sites incorporated or with added sequences overlapping with the 5′ and 3′ ends of the linearized E. coli expression vector. With restriction enzyme digestion and ligation or homolog recombination based cloning, the PCR products were cloned into selected E. coli expression vectors, i.e. pET16b with N-His tag, pET24a with C-His tag or without tag. In case of co-expression of two proteins for a binary (IPD092-1/2, IPD095-1/2, IPD100-1/2, IPD106-1/2) and three proteins for tripartite (IPD099-1/-2/-3) toxins, their native operon sequences were also cloned into one of the E. coli vectors. The proteins were expressed in BL21(DE3), C41 or SHuffle® E. coli host cells with 1 mM IPTG overnight induction at 16° C. The recombinant protein was extracted from E. coli culture after induction. Cleared cell lysates or purified proteins were assayed on insect targets as described in Example 1.

### Example 7

### Identification of Homologs

Genomic DNA was extracted from various internal strains, the species was identified and the genome was sequenced as described in Example 3. Gene identities may be determined by conducting BLAST® (Basic Local Alignment 20 Search Tool; Altschul, et al., (1993) J. Mol. Biol. 215:403-410; see also ncbi.nlm.nih.gov/BLAST/, which can be accessed using the www prefix) searches under default parameters for similarity to sequences contained in the internal genomes and in the publicly available BLAST® “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The polynucleotide sequences of SEQ ID NO: SEQ ID NO: 546, SEQ ID NO: 547, SEQ ID NO: 562, SEQ ID NO: 563, SEQ ID NO: 590, SEQ ID NO: 591, SEQ ID NO: 592, SEQ ID NO: 593, SEQ ID NO: 611, SEQ ID NO: 612, SEQ ID NO: 614, SEQ ID NO: 617, SEQ ID NO: 618, SEQ ID NO: 621, SEQ ID NO: 629, SEQ ID NO: 635 were analyzed.

Table 4 shows the IPD092-1Aa and IPD092-2Aa polypeptide homologs identified, sequence identification numbers for each and the bacterial isolates they were identified from.

Table 5 shows a matrix table of pair-wise identity relationships for global alignments of the IPD092Aa-1 homologs, based upon the Needleman-Wunsch algorithm, as implemented in the Needle program (EMBOSS tool suite).

Table 6 shows a matrix table of pair-wise identity relationships for global alignments of the IPD092Aa-1 homologs, based upon the Needleman-Wunsch alogorithm, as implemented in the Needle program (EMBOSS tool suite).

TABLE 4

| | Identity to | | | | | |
|---|---|---|---|---|---|---|
| | IPD092-1Aa or | | | | | |
| IPD Name | IPD092-2Aa | Source | Species | activity | protein | DNA |
| IPD092-1Aa | | internal strain - | Pseudomonas | WCRW | SEQ ID NO: 1 | SEQ ID NO: 546 |
| | | SSP473A12-1; JH17494-4; | rhodesiae | | | |
| | | JH17565-4; JH17574-1; | | | | |
| | | JH17681-1; JH17728-1; | | | | |
| | | JH17729-3; JH17730-1; | | | | |
| | | JH17731-2; JH31230-2; | | | | |
| | | SSP4608B2a; JH17728-1 | | | | |
| IPD092-2Aa | | internal strain - | Pseudomonas | WCRW | SEQ ID NO: 2 | SEQ ID NO: 547 |
| | | SSP473A12-1; JH17494-4; | rhodesiae | | | |
| | | JH17565-4; JH17494-4; | | | | |
| | | JH17565-4; JH17574-1; | | | | |
| | | JH17581-1; JH17728-1; | | | | |
| | | JH17729-3; JH17730-1; | | | | |
| | | JH17731-2; JH31230-2 | | | | |
| IPD092-1Ab | 95.7% | internal strain SSP535F3b | Pseudomonas | | SEQ ID NO: 3 | |
| | | | rhodesiae | | | |
| IPD092-2Ab | 96.9% | internal strain SSP535F3b | Pseudomonas | | SEQ ID NO: 4 | |
| | | | rhodesiae | | | |
| IPD092-1Ba | 89.1% | internal strain SSP743C9-1 | Pseudomonas frederiksbergensis | | SEQ ID NO: 5 | |
| IPD092-2Ba | 83.4% | internal strain SSP743C9-1 | Pseudomonas frederiksbergensis | | SEQ ID NO: 6 | SEQ ID NO: 548 |
| IPD092-1Bb | 81.3% | Internal strain SS43D2 | Pseudomonas frederiksbergensis | | SEQ ID NO: 7 | SEQ ID NO: 549 |
| IPD092-2Bb | 78.8% | Internal strain SS43D2 | Pseudomonas frederiksbergensis | | SEQ ID NO: 8 | |
| IPD092-1Ca | 76.4% | internal strain SSP616E3-1 | Pseudomonas | | SEQ ID NO: 9 | SEQ ID NO: 550 |
| | | | fluorescens | | | |
| IPD092-2Ca | 73.1% | internal strain SSP616E3-1 | Pseudomonas | | SEQ ID NO: 10 | SEQ ID NO: 551 |
| | | | fluorescens | | | |
| IPD092-1Cb | 71.0% | internal strain SSP642E9-1 | Pseudomonas | WCRW | SEQ ID NO: 11 | SEQ ID NO: 552 |
| | | | protegens | | | |
| IPD092-2Cb | 68.9% | internal strain SSP642E9-1 | Pseudomonas | WCRW | SEQ ID NO: 12 | SEQ ID NO: 553 |
| | | | protegens | | | |
| IPD092-1Da | 65.4% | internal strain JH67425-2 | Pseudomonas | | SEQ ID NO: 13 | SEQ ID NO: 554 |
| | | | protegens | | | |
| IPD092-2Da | 61.4% | internal strain JH67425-2 | Pseudomonas | | SEQ ID NO: 14 | SEQ ID NO: 555 |
| | | | protegens | | | |
| IPD092-1Db | 61.1% | internal strainJH94099-1 | Chromobacterium | | SEQ ID NO: 15 | |
| | | | aquaticum | | | |
| IPD092-2Db | 54.5% | internal strainJH94099-1 | Chromobacterium | | SEQ ID NO: 16 | |
| | | | aquaticum | | | |
| IPD092-1Ea | 54.7% | internal strain JH90668-1 | Chromobacterium | | SEQ ID NO: 17 | SEQ ID NO: 556 |
| | | | aquaticum | | | |
| IPD092-2Ea | 54.0% | internal strain JH90668-1 | Chromobacterium | | SEQ ID NO: 18 | SEQ ID NO: 557 |
| | | | aquaticum | | | |
| IPD092-1Eb | 50.5% | internal strain SSP283D11-1 | Burkholderia ambifaria | | SEQ ID NO: 19 | |
| IPD092-2Eb | 49.0% | internal strain SSP283D11-1 | Burkholderia ambifaria | | SEQ ID NO: 20 | |
| IPD092-2Ec | 55.8% | SSP932E4-1 | Pseudomonas mosselii | | SEQ ID NO: 21 | |
| IPD092-1Fa | 41.5% | internal strain SSP588G7-1 | Pseudomonas | | SEQ ID NO: 22 | SEQ ID NO: 558 |
| | | | putida | | | |
| IPD092-2Fa | 41.8% | internal strain SSP588G7-1 | Pseudomonas | | SEQ ID NO: 23 | SEQ ID NO: 559 |
| | | | putida | | | |
| IPD092-1Fb | 42.9% | internal strain SSP603E4-1 | Pseudomonas vranovensis | WCRW | SEQ ID NO: 24 | SEQ ID NO: 560 |
| IPD092-2Fb | 44.9% | internal strain SSP603E4-1 | Pseudomonas vranovensis | WCRW | SEQ ID NO: 25 | SEQ ID NO: 561 |
| IPD092-1Fc | 45.9% | SSP932E4-1 | Pseudomonas mosselii | | SEQ ID NO: 26 | |

TABLE 5

| | IPD092- | IPD092- | IPD092- | IPD092- | IPD092- | IPD092- |
|---|---|---|---|---|---|---|
| | 1Ab SEQ | 10a SEQ | 18b SEQ | 1Ca SEQ | 1Cb SEQ | 1Da SEQ |
| | ID NO: 3 | ID NO: 5 | ID NO: 7 | ID NO: 9 | ID NO: 11 | ID NO: 13 |
| IPD092-1Aa | 95.7 | 89.1 | 82.2 | 76.4 | 71.5 | 65.9 |
| SEQ ID NO: 1 | | | | | | |
| IPD092-1Ab | — | 88.6 | 81.8 | 75.9 | 72.4 | 65.9 |
| SEQ ID NO: 3 | | | | | | |
| IPD092-1Ba | — | — | 87.0 | 77.9 | 71.5 | 67.5 |
| SEQ ID NO: 5 | | | | | | |
| IPD092-1Bb | — | — | — | 77.9 | 69.6 | 64.8 |
| SEQ ID NO: 7 | | | | | | |
| IPD092-1Ca | — | — | — | — | 68.4 | 64.9 |
| SEQ ID NO: 9 | | | | | | |
| IPD092-1Cb | — | — | — | — | — | 71.5 |
| SEQ ID NO: 11 | | | | | | |
| IPD092-1Da | — | — | — | — | — | — |
| SEQ ID NO: 13 | | | | | | |
| IPD092-1Db | — | — | — | — | — | — |
| SEQ ID NO: 15 | | | | | | |
| IPD092-1Ea | — | — | — | — | — | — |
| SEQ ID NO: 17 | | | | | | |
| IPD092-1Eb | — | — | — | — | — | — |
| SEQ ID NO: 19 | | | | | | |
| IPD092-1Fa | — | — | — | — | — | — |
| SEQ ID NO: 22 | | | | | | |
| IPD092-1Fb | — | — | — | — | — | — |
| SEQ ID NO: 24 | | | | | | |
| | IPD092- | IPD092- | IPD092- | IPD092- | IPD092- | IPD092- |
| | 1Db SEQ | 1Ea SEQ | 1Eb SEQ | 1Fa SEQ | 1Fb SEQ | 1Fc SEQ |
| | ID NO: 15 | ID NO: 17 | ID NO: 19 | ID NO: 22 | ID NO: 24 | ID NO: 26 |
| IPD092-1Aa | 61.1 | 54.7 | 52.6 | 47.0 | 45.1 | 33.3 |
| SEQ ID NO: 1 | | | | | | |
| IPD092-1Ab | 59.4 | 53.1 | 50.2 | 45.8 | 46.0 | 33.0 |
| SEQ ID NO: 3 | | | | | | |
| IPD092-1Ba | 58.0 | 52.1 | 50.7 | 45.9 | 46.8 | 32.2 |
| SEQ ID NO: 5 | | | | | | |
| IPD092-1Bb | 57.9 | 52.1 | 48.4 | 44.9 | 45.6 | 32.9 |
| SEQ ID NO: 7 | | | | | | |
| IPD092-1Ca | 59.0 | 53.5 | 48.6 | 47.4 | 42.5 | 33.6 |
| SEQ ID NO: 9 | | | | | | |
| IPD092-1Cb | 56.5 | 54.0 | 49.1 | 45.4 | 43.4 | 35.9 |
| SEQ ID NO: 11 | | | | | | |
| IPD092-1Da | 54.0 | 52.4 | 48.4 | 46.5 | 42.3 | 35.0 |
| SEQ ID NO: 13 | | | | | | |
| IPD092-1Db | — | 79.7 | 48.4 | 41.3 | 42.6 | 33.9 |
| SEQ ID NO: 15 | | | | | | |
| IPD092-1Ea | — | — | 46.3 | 38.3 | 37.8 | 30.6 |
| SEQ ID NO: 17 | | | | | | |
| IPD092-1Eb | — | — | — | 38.5 | 40.0 | 29.3 |
| SEQ ID NO: 19 | | | | | | |
| IPD092-1Fa | — | — | — | — | 53.0 | 55.2 |
| SEQ ID NO: 22 | | | | | | |
| IPD092-1Fb | — | — | — | — | — | 41.6 |
| SEQ ID NO: 24 | | | | | | |

TABLE 6

| | IPD092- | IPD092- | IPD092- | IPD092- | IPD092- | IPD092- |
|---|---|---|---|---|---|---|
| | 2Ab SEQ | 2Ba SEQ | 2Bb SEQ | 2Ca SEQ | 2Cb SEQ | 2Da SEQ |
| | ID NO: 4 | ID NO: 6 | ID NO: 8 | ID NO: 10 | ID NO: 12 | ID NO: 14 |
| IPD092-2Aa | 96.9 | 85.6 | 80.9 | 73.1 | 69.6 | 62.4 |
| SEQ ID NO: 2 | | | | | | |
| IPD092-2Ab | — | 85.1 | 80.9 | 73.1 | 69.6 | 60.9 |
| SEQ ID NO: 4 | | | | | | |
| IPD092-2Ba | — | — | 87.6 | 71.0 | 70.6 | 61.9 |
| SEQ ID NO: 6 | | | | | | |
| IPD092-2Bb | — | — | — | 74.1 | 70.6 | 62.9 |
| SEQ ID NO: 8 | | | | | | |
| IPD092-2Ca | — | — | — | — | 66.8 | 59.9 |
| SEQ ID NO: 10 | | | | | | |
| IPD092-2Cb | — | — | — | — | — | 68.3 |
| SEQ ID NO: 12 | | | | | | |
| IPD092-2Da | — | — | — | — | — | — |
| SEQ ID NO: 14 | | | | | | |
| IPD092-2Db | — | — | — | — | — | — |
| SEQ ID NO: 16 | | | | | | |
| IPD092-2Ea | — | — | — | — | — | — |
| SEQ ID NO: 18 | | | | | | |
| IPD092-2Eb | — | — | — | — | — | — |
| SEQ ID NO: 20 | | | | | | |
| IPD092-2Ec | — | — | — | — | — | — |
| SEQ ID NO: 21 | | | | | | |
| IPD092-2Fa | — | — | — | — | — | — |
| SEQ ID NO: 23 | | | | | | |
| | IPD092- | IPD092- | IPD092- | IPD092- | IPD092- | IPD092- |
| | 2Db SEQ | 2Ea SEQ | 2Eb SEQ | 2Ec SEQ | 2Fa SEQ | 2Fb SEQ |
| | ID NO: 16 | ID NO: 18 | ID NO: 20 | ID NO: 21 | ID NO: 23 | ID NO: 25 |
| IPD092-2Aa | 55.7 | 55.2 | 49.0 | 38.3 | 41.8 | 43.7 |
| SEQ ID NO: 2 | | | | | | |
| IPD092-2Ab | 57.2 | 56.7 | 47.9 | 33.3 | 41.8 | 45.9 |
| SEQ ID NO: 4 | | | | | | |
| IPD092-2Ba | 57.5 | 57.7 | 50.3 | 34.7 | 44.0 | 42.7 |
| SEQ ID NO: 6 | | | | | | |
| IPD092-2Bb | 56.0 | 55.2 | 48.7 | 34.7 | 43.0 | 41.7 |
| SEQ ID NO: 8 | | | | | | |
| IPD092-2Ca | 53.0 | 52.5 | 46.9 | 33.9 | 41.5 | 39.2 |
| SEQ ID NO: 10 | | | | | | |
| IPD092-2Cb | 55.2 | 54.5 | 50.0 | 31.7 | 42.8 | 44.7 |
| SEQ ID NO: 12 | | | | | | |
| IPD092-2Da | 58.5 | 56.1 | 52.2 | 31.7 | 41.9 | 42.9 |
| SEQ ID NO: 14 | | | | | | |
| IPD092-2Db | — | 91.5 | 51.0 | 33.6 | 39.5 | 41.4 |
| SEQ ID NO: 16 | | | | | | |
| IPD092-2Ea | — | — | 53.7 | 31.9 | 39.1 | 38.4 |
| SEQ ID NO: 18 | | | | | | |
| IPD092-2Eb | — | — | — | 47.4 | 33.3 | 32.3 |
| SEQ ID NO: 20 | | | | | | |
| IPD092-2Ec | — | — | — | — | 57.9 | 44.6 |
| SEQ ID NO: 21 | | | | | | |
| IPD092-2Fa | — | — | — | — | — | 56.0 |
| SEQ ID NO: 23 | | | | | | |

Table 7 shows the IPD095-1Aa and IPD095-2Aa polypeptide homologs identified, sequence identification numbers for each and the bacterial isolates they were identified from.

TABLE 7

| | Identity to | | | | | |
|---|---|---|---|---|---|---|
| | IPD095- | | | | | |
| IPD Name | 1/2Aa | Source | Species | activity | Protein | DNA |
| IPD095-1Aa | | SSP232H12-1, SSP237H1d | Serratia nematodiphila | WCRW | SEQ ID NO: 27 | SEQ ID NO: 562 |
| IPD095-2Aa | | SSP232H12-1, SSP237H1d | Serratia nematodiphila | WCRW | SEQ ID NO: 28 | SEQ ID NO: 563 |
| IPD095-1Ab | 98.9% | Internal strain SSP587F12-1 | Serratia marcescens | | SEQ ID NO: 29 | SEQ ID NO: 564 |
| IPD095-1Ac | 97.7% | Internal strain JH20487-2; | Serratia marcescens | | SEQ ID NO: 30 | SEQ ID NO: 565 |
| | | JH52720-2; JH52735-2 | | | | |
| IPD095-1Ad | 97.1% | NCBI_WP_019455460; | Serratia marcescens | | SEQ ID NO: 31 | SEQ ID NO: 566 |
| | | internal - JH47141-1 (2aa); | H1q | | | |
| | | JH47215-1 (2aa) | | | | |
| IPD095-1Ae | 97% | NCBI_A0A0A5TBG6 | Serratia marcescens | | SEQ ID NO: 32 | |
| IPD095-1Af | 98% | NCBI_A0A0A5NMY4 | Serratia marcescens | | SEQ ID NO: 33 | |
| IPD095-1Ag | 98.3% | internal strain SSP639G4-2; | Serratia marcescens | | SEQ ID NO: 34 | |
| | | SSP639F11-1 | | | | |
| IPD095-1Ah | 97.1% | 4714-1 | | | SEQ ID NO: 35 | |
| IPD095-1Ai | 99.4% | WP_033650308 | | | SEQ ID NO: 36 | |
| IPD095-1Aj | 98.3% | WP_047026045 | | | SEQ ID NO: 37 | |
| IPD095-1Ak | 96.6% | A0A0A5NMY4 | | | SEQ ID NO: 38 | |
| IPD095-1Al | 97.1% | WP_055313380 | | | SEQ ID NO: 39 | |
| IPD095-1Am | 97.7% | WP_063919633 | Serratia sp. | | SEQ ID NO: 40 | |
| IPD095-1Ba | 85.1% | internal strain JH20785-4; | Serratia proteamaculans | | SEQ ID NO: 41 | SEQ ID NO: 567 |
| | | JH78168-2 (1aa); | | | | |
| IPD095-1Bb | 83.9% | internal strain JH21591-1; | Serratia liquefaciens | | SEQ ID NO: 42 | SEQ ID NO: 568 |
| | | JH21602-1 | | | | |
| IPD095-1Bc | 82.2% | internal strain SSP443E1-2; | Serratia plymuthica | | SEQ ID NO: 43 | SEQ ID NO: 569 |
| | | SSP443E10-1; JH79545-2; | | | | |
| | | JH90222-1; KL1 pooled | | | | |
| IPD095-1Bd | 83.3% | NCBI_WP_017892809 | Serratia sp. S4 | | SEQ ID NO: 44 | |
| IPD095-1Be | 82.2% | NCBI_YP_008159223 | Serratia plymuthica | | SEQ ID NO: 45 | SEQ ID NO: 570 |
| | | | S13 | | | |
| IPD095-1Bf | 84% | NCBI-WP_041418562 | Serratia proteamaculans | | SEQ ID NO: 46 | |
| IPD095-1Bg | 86% | WP_044551028 | Serratia liquefaciens | | SEQ ID NO: 47 | |
| IPD095-1Bh | 83.3% | internal pooled XM16 | | | SEQ ID NO: 48 | |
| IPD095-1Bi | 84% | internal pool HK- | | | SEQ ID NO: 49 | |
| | | 8_D1470007_NODE_3_6056 | | | | |
| IPD095-1Bj | 84.5% | JH78168-2 | | | SEQ ID NO: 50 | |
| IPD095-1Bk | 81% | KL1_pooled_NODE_1076_116 | | | SEQ ID NO: 51 | |
| IPD095-1Bl | 81.6% | WP_062870795 | | | SEQ ID NO: 52 | |
| IPD095-1Bm | 81.6% | WP_063198351 | Serratia plymuthica | | SEQ ID NO: 53 | |
| IPD095-1Ca | 79.9% | internal strain - JH19892-1 | Serratia plymuthica | | SEQ ID NO: 54 | SEQ ID NO: 571 |
| IPD095-1Cb | 79.3% | NCBI_WP_006318834 | Serratia plymuthica | | SEQ ID NO: 55 | SEQ ID NO: 572 |
| | | | PRI-2C | | | |
| IPD095-1Cc | 79% | internal pool XM4 | | | SEQ ID NO: 56 | |
| IPD095-1Cd | 79.3% | PMCJ3367H8-1 | Serratia plymuthica | | SEQ ID NO: 57 | SEQ ID NO: 573 |
| IPD095-1Ea | 52.8% | NCBI-WP_027273487.1 | Leminorella grimontii | | SEQ ID NO: 58 | SEQ ID NO: 574 |
| | | hypothetical protein | | | | |
| IPD095-2Ab | 97.9% | internal strain SSP587F12-1 | Serratia marcescens | | SEQ ID NO: 59 | SEQ ID NO: 575 |
| IPD095-2Ac | 98.9% | internal strain JH20487-2 | Serratia nematodiphil | | SEQ ID NO: 60 | SEQ ID NO: 576 |
| IPD095-2Ae | 99.3% | internal stain JH52720-2; | Serratia marcescens | | SEQ ID NO: 61 | SEQ ID NO: 577 |
| | | JH52935-2 | | | | |
| IPD095-2Af | 99.4% | NCBI_EZQ65351 | Serratia marcescens | | SEQ ID NO: 62 | SEQ ID NO: 578 |
| | | | BIDMC 81 | | | |
| IPD095-2Ag | 97% | internal - JH4714-1; | Serratia marcescens | | SEQ ID NO: 63 | |
| | | JH47215-1 | | | | |
| IPD095-2Ah | 96% | NCBI_A0A0A5LR46 | Serratia marcescens | | SEQ ID NO: 64 | |
| IPD095-2Aj | 98% | NCBI_A0A0A5VA11 | Serratia marcescens | | SEQ ID NO: 65 | |
| IPD095-2Aj | 98.9% | internal strain SSP639G4-2; | Serratia marcescens | | SEQ ID NO: 66 | |
| | | SSP639F11-1; | | | | |
| IPD095-2Ak | 97.2% | AKL43846 | Serratia marcescens | | SEQ ID NO: 67 | |
| IPD095-2Al | 98.9% | internal pooled XM13 | | | SEQ ID NO: 68 | |
| IPD095-2Am | 96.8% | WP_055316991 | Serratia marcescens | | SEQ ID NO: 69 | |
| IPD095-2An | 97.6% | WP_060439867 | Serratia marcescens | | SEQ ID NO: 70 | |
| IPD095-2Ae | 96.4% | WP_060435039 | Serratia marcescens | | SEQ ID NO: 71 | |
| IPD095-2Ap | 98.3% | WP_047026044 | | | SEQ ID NO: 72 | |
| IPD095-2Aq | 97% | WP_055313378 | | | SEQ ID NO: 73 | |
| IPD095-2Ar | 98.3% | WP_046898260 | | | SEQ ID NO: 74 | |
| IPD095-2As | 99.1% | XM28_pooled_NODE_1742_74 | | | SEQ ID NO: 75 | |
| IPD095-2At | 97.2% | WP_060418690 | | | SEQ ID NO: 76 | |
| IPD095-2Au | 97.2% | PMC3675E4-1 | Serratia marcescens | | SEQ ID NO: 77 | |
| IPD095-2Av | 98.1% | PMC3703F6-1 | Serratia ureilytica | | SEQ ID NO: 78 | |
| IPD095-2Aw | 97.2% | SAY43294 | Serratia marcescens | | SEQ ID NO: 79 | |
| IPD095-2Ca | 78.9% | NCBI_WP_019455461 | Serratia marcescens | | SEQ ID NO: 80 | SEQ ID NO: 579 |
| | | | H1q | | | |
| IPD095-2Cb | 79.8% | WP_063919534 | Serratia sp. | | SEQ ID NO: 81 | |
| IPD095-2Da | 62.1% | internal strain JH20785-4 | Serratia proteamaculans | | SEQ ID NO: 82 | SEQ ID NO: 580 |
| IPD095-2Db | 62.2% | internal strain JH21591-1; | Serratia liquefaciens | | SEQ ID NO: 83 | SEQ ID NO: 581 |
| | | JH21602-1; JH78168-2 (2aa); | | | | |
| IPD095-2Dc | 62.6% | internal strain | Serratia plymuthica | | SEQ ID NO: 84 | SEQ ID NO: 582 |
| | | SSP443E1-2 | | | | |
| IPD095-2Dd | 62.8% | internal strain SSP443E10-1; | Serratia plymuthica | | SEQ ID NO: 85 | SEQ ID NO: 583 |
| | | JH79545-2 (1aa); JH80222-1 | | | | |
| | | (1aa) | | | | |
| IPD095-2De | 63.2% | internal strain JH19892-1 | Serratia plymuthica | | SEQ ID NO: 86 | SEQ ID NO: 584 |
| IPD095-2Df | 62.4% | NCBI_WP_017892808 | | | SEQ ID NO: 87 | |
| IPD095-2Dg | 62.2% | NCBI_WP_006318833 | Serratia plymuthica | | SEQ ID NO: 88 | SEQ ID NO: 585 |
| | | | PRI-2C | | | |
| IPD095-2Dh | 63.6% | NCBI_YP_001478610 | Serratia proteamaculans 568 | | SEQ ID NO: 89 | SEQ ID NO: 586 |
| IPD095-2Di | 62.2% | NCBI_YP_0081592222 | Serratia plymuthica S13 | | SEQ ID NO: 90 | SEQ ID NO: 587 |
| IPD095-2Dj | 61.9% | NCBI_WP_006325190 | Serratia plymuthica | | SEQ ID NO: 91 | SEQ ID NO: 588 |
| | | | A30 | | | |
| IPD095-2Dk | 62.4% | NCBI_AHY07405.1 | Serratia plymuthica | | SEQ ID NO: 92 | SEQ ID NO: 589 |
| | | hypothetical protein | V4 | | | |
| IPD095-2Dl | 62% | WP_044551026 | Serratia liquefaciens | | SEQ ID NO: 93 | |
| IPD095-2Dm | 62% | internal pooled KL1 | | | SEQ ID NO: 94 | |
| IPD095-2Dn | 63% | internal pooled KL1 | | | SEQ ID NO: 95 | |
| IPD095-2Do | 63.4% | internal pooled XM8 | | | SEQ ID NO: 96 | |
| IPD095-2Dp | 61.9% | internal pooled XM16 | | | SEQ ID NO: 97 | |
| IPD095-2Dq | 61.9% | WP_062790859 | Serratia sp. | | SEQ ID NO: 98 | |
| IPD095-2Dr | 62.1% | JH78168-2 | | | SEQ ID NO: 99 | |
| IPD095-2Ds | 62.8% | JH79545-2 | | | SEQ ID NO: 100 | |
| IPD095-2Dt | 63% | WP_062871340 | | | SEQ ID NO: 101 | |
| IPD095-2Du | 63% | WP_063198354 | Serratia plymuthica | | SEQ ID NO: 102 | |
| IPD095-2Dv | 62.1% | ANK01150 | Serratia plymuthica | | SEQ ID NO: 103 | |
| IPD095-2Dw | 62.4% | WP_073439915 | Serratia plymuthica | | SEQ ID NO: 104 | |
| IPD095-2Ea | 51.5% | NCBI_WP_005186718, | | | SEQ ID NO: 105 | |
| | | WP_050008601, | | | | |
| IPD095-2Eb | 50% | NCBI-WP_035346074, | Dickeya sp. | | SEQ ID NO: 106 | |
| | | WP_051124215 | | | | |
| IPD095-2Ec | 58.1% | internal pooled XM10 | | | SEQ ID NO: 107 | |
| IPD095-2Ed | 57.6% | WP_047607950 | Rahnella aquatilis | | SEQ ID NO: 108 | |
| IPD095-2Ee | 57.2% | WP_047611350 | Rahnella aquatilis | | SEQ ID NO: 109 | |
| IPD095-2Ef | 51.2% | A0A0E8JDU8, | Yersinia intermedia | | SEQ ID NO: 110 | |
| | | WP_050882131 | | | | |
| IPD095-2Eg | 61.4% | WP_061495529 | Enterobacter sp. | | SEQ ID NO: 111 | |
| IPD095-2Eh | 51.7% | WP_042568849 | | | SEQ ID NO: 112 | |
| IPD095-2Ei | 51.5% | WP_0500088601 | | | SEQ ID NO: 113 | |
| IPD095-2Ej | 51.5% | WP_050074011 | | | SEQ ID NO: 114 | |
| IPD095-2Ek | 50% | WP_051124215 | | | SEQ ID NO: 115 | |
| IPD095-2El | 51.3% | WP_050882131 | | | SEQ ID NO: 116 | |
| IPD095-2Em | 55.3% | WP_067707065 | Erwinia sp. | | SEQ ID NO: 117 | |
| IPD095-2Fa | 47.0% | NCBI_WP_017346989 | | | SEQ ID NO: 118 | |
| IPD095-2Fb | 47.8% | XM28_pooled_NODE_127_1270 | | | SEQ ID NO: 119 | |
| IPD095-2Fc | 47.7% | A0A1C4CX92 | Enterobacter | | SEQ ID NO: 120 | |
| | | | oryzendophyticus | | | |

Table 8 shows the IPD097-1Aa polypeptide homologs identified, sequence identification numbers for each and the bacterial isolates they were identified from.

TABLE 8

| | Identity to | | | | | |
|---|---|---|---|---|---|---|
| IPD Name | IPD097Aa | Source | Species | activity | Protein | DNA |
| IPD097Aa | | internal strain JH58776-1, | Haemophilus piscium | WCRW | SEQ ID NO: 121 | SEQ ID NO: 590 |
| | | JH67351-1 (1aa differ); | | | | |
| | | JH78490-2; JH82751-1; XM5 pooled | | | | |
| IPD097Ab | 96.6% | NCBI YP_001143082.1 | Aeromonas salmonicida | | SEQ ID NO: 122 | |
| | | hypothetical protien | | | | |
| IPD097Ac | 96.9% | internal strain SSP651B-1 | Aeromonas salmonicida | | SEQ ID NO: 123 | |
| IPD097Ad | 96% | NCBI-WP_017411752 | Aeromonas salmonicida | | SEQ ID NO: 124 | |
| IPD097Ae | 98% | WP_042860997 | Aeromonas piscicola | | SEQ ID NO: 125 | |
| IPD097Af | 90.4% | WP_050718115 | Aeromonas tecta | | SEQ ID NO: 126 | |
| IPD097Ag | 99.3% | JH67351-1; PMCH4138E11-1; | | | SEQ ID NO: 127 | |
| | | PMCH4138E05-1 | | | | |
| IPD097Ah | 99.3% | XM5_pooled_NODE_966_119 | | | SEQ ID NO: 128 | |
| IPD097Ai | 99.3% | JH78147-1 | | | SEQ ID NO: 129 | |
| IPD097Aj | 95.2% | WP_058393614 | | | SEQ ID NO: 130 | |
| IPD097Ak | 95.9% | AA0W0AXB2 | | | SEQ ID NO: 131 | |
| IPD097Al | 96.2% | XM26_pooled_NODE_540_188 | | | SEQ ID NO: 132 | |
| IPD097Am | 98.6% | WP_065403351 | Aeromonas piscicola | | SEQ ID NO: 133 | |
| IPD097Ba | 82.9% | NCBI WP_005348511.1 | Aeromonas diversa | | SEQ ID NO: 134 | |
| | | hypothetical | | | | |
| | | protein | | | | |
| IPD097Ea | 56.6% | WP_066501527 | Clostridiales bacterium | | SEQ ID NO: 135 | |

Table 9 shows the IPD099-1Aa, IPD099-2Aa, and IPD099-3Aa polypeptide homologs identified, sequence identification numbers for each and the bacterial isolates they were identified from.

TABLE 9

| | Identity to | | | | | |
|---|---|---|---|---|---|---|
| | IPD099- | | | | | |
| IPD Name | 1/2/3Aa | Source | Species | activity | Protein | DNA |
| IPD099-1Aa | | internal strain JH58776-1; | Aeromonas salmonicida | WCRW | SEQ ID NO: 136 | SEQ ID |
| | | JH91676-2 | ssp. Salmonicida | | | NO: 591 |
| IPD099-2Aa | | internal strain JH28776-1, | Aeromonas salmonicida | WCRW | SEQ ID NO: 137 | SEQ ID |
| | | JH67351-1 (1aa), NCBI | ssp. Salmonicida | | | NO: 592 |
| | | KFN17897.1; JH78490-2; | | | | |
| | | WP_042869733; | | | | |
| | | WP_043556154 | | | | |
| IPD099-3Aa | | JH58776-1 | Aeromonas salmonicida | WCRW | SEQ ID NO: 138 | SEQ ID |
| | | | ssp. Salmonicida | | | NO: 593 |
| IPD099-1Ab | 98% | JH82751-1 (2aa differ) | Aeromonas salmonicida | WCRW | SEQ ID NO: 139 | SEQ ID |
| | | | | | | NO: 594 |
| IPD099-1Ac | 98% | JH67351-1 (3aa differ) | Aeromonas salmonicida | WCRW | SEQ ID NO: 140 | SEQ ID |
| | | | | | | NO: 595 |
| IPD099-1Ad | 97% | NCBI KFN17896.1 | Aeromonas salmonicida | | SEQ ID NO: 141 | |
| IPD099-1Ae | 99% | JH78490-2 | Haemophilus piscium | WCRW | SEQ ID NO: 142 | SEQ ID |
| | | | | | | NO: 596 |
| IPD099-1Af | 92% | WP_042036788 | Aeromonas popoffii | | SEQ ID NO: 143 | |
| IPD099-1Ag | 98% | internal pooled XM5 | | | SEQ ID NO: 144 | |
| IPD099-1Ah | 99% | internal pooled KL1 | | | SEQ ID NO: 145 | |
| IPD099-1Ai | 96.2% | WP_042869735 | Aeromonas piscicola | | SEQ ID NO: 146 | |
| IPD099-1Aj | 98.4% | WP_043556153 | Aeromonas bestiarum | | SEQ ID NO: 147 | |
| IPD099-1Ak | 98.4% | internal pooled XM23 | | | SEQ ID NO: 148 | |
| IPD099-1Al | 99.7% | JH91676-2 | | | SEQ ID NO: 149 | |
| IPD099-1Am | 99.2% | JH78147-1 | | | SEQ ID NO: 150 | |
| IPD099-1An | 95.9% | WP_065403857 | Aeromonas piscicola | | SEQ ID NO: 151 | |
| IPD099-1Ba | 80% | NCBI FW306009.1 | Aeromonas hydrophila | | SEQ ID NO: 152 | |
| IPD099-1Bb | 80% | NCBI WP_011706401.1 | Aeromonas hydrophila | | SEQ ID NO: 153 | |
| IPD099-1Ca | 79% | NCBI WP_017786782.1 | Aeromonas hydrophila | | SEQ ID NO: 154 | |
| IPD099-1Cb | 79% | K01-14A1-1, K01-20A3-2 | Aeromonas hydrophila | | SEQ ID NO: 155 | |
| IPD099-1Cc | 79% | JH58748-2; XM5 pooled; | Aeromonas hydrophila | WcRw | SEQ ID NO: 156 | SEQ ID |
| | | NCBI-AJQ55024 | | | | NO: 597 |
| IPD099-1Cd | 79% | JH67338-1 | Aeromonas hydrophila | WCRW | SEQ ID NO: 157 | SEQ ID |
| | | | | | | NO: 598 |
| IPD099-1Ce | 79% | JH71362-2, JH70211-1 | Aeromonas hydrophila | WCRW | SEQ ID NO: 158 | SEQ ID |
| | | | | | | NO: 599 |
| IPD099-1Cf | 79% | NCBI WP_019840111.1 | Aeromonas sp. MDS6 | | SEQ ID NO: 159 | |
| IPD099-1Cg | 79% | NCBI WP_WP_017764245.1 | Aeromonas hydrophila | | SEQ ID NO: 160 | |
| IPD099-1Ch | 79% | NCBI WP_024941132.1 | Aeromonas hydrophila | | SEQ ID NO: 161 | |
| IPD099-1Ci | 79% | NCBI WP_010633676.1 | Aeromonas dhakensis | | SEQ ID NO: 162 | |
| IPD099-1Cj | 79% | NCBI WP_017778854.1 | Aeromonas hydrophila | | SEQ ID NO: 163 | |
| IPD099-1Ck | 79% | NCBI WP_017782950.1 | Aeromonas hydrophila | | SEQ ID NO: 164 | |
| IPD099-1Cl | 79% | NCBI WP_017408110.1 | Aeromonas hydrophila | | SEQ ID NO: 165 | |
| IPD099-1Cm | 79% | NCBI WP_005302106.1 | Aeromonas | | SEQ ID NO: 166 | |
| IPD099-1Cn | 79% | NCBI WP_006302106.1 | Aeromonas | | SEQ ID NO: 167 | |
| IPD099-1Co | 79% | NCBI KER63311.1 | Aeromonas hydrophila | | SEQ ID NO: 168 | |
| IPD099-1Cp | 78% | NCBI WP_029300415.1 | Aeromonas hydrophila | | SEQ ID NO: 169 | |
| IPD099-1Cq | 78% | NCBI EXH80101.1 | Aeromonas hydrophila | | SEQ ID NO: 170 | |
| IPD099-1Cr | 78% | NCBI WP_029302783.1 | Aeromonas hydrophila | | SEQ ID NO: 171 | |
| IPD099-1Cs | 77% | NCBI WP_016351060.1 | Aeromonas hydrophila | | SEQ ID NO: 172 | |
| IPD099-1Ct | 79% | WP_039213462; internal | Aeromonas hydrophila | | SEQ ID NO: 173 | |
| | | pooled XM1 | | | | |
| IPD099-1Cu | 79% | NCBI - A0A0A6C9G1 | Aeromonas hydrophila | | SEQ ID NO: 174 | |
| IPD099-1Cv | 79% | WP_042007673 | Aeromonas dhakensis | | SEQ ID NO: 175 | |
| IPD099-1Cw | 79% | NCBI - A0A0A5LAG3 | Aeromonas hydrophila | | SEQ ID NO: 176 | |
| IPD099-1Cx | 79% | JH78710-1 | Aeromonas hydrophila | | SEQ ID NO: 177 | |
| IPD099-1Cy | 79% | JH99577-2; | Aeromonas hydrophila | | SEQ ID NO: 178 | |
| | | WP_049047798 | | | | |
| IPD099-1Cz | 79% | internal pooled XM1 | | | SEQ ID NO: 179 | |
| IPD099-1Caa | 79% | internal pooled KL1 | | | SEQ ID NO: 180 | |
| IPD099-1Cab | 79% | WP_043169496 | Aeromonas | | SEQ ID NO: 181 | |
| IPD099-1Cac | 78.8% | WP_045527022; | Aeromonas hydrophila | | SEQ ID NO: 182 | |
| | | WP_045789543 | | | | |
| IPD099-1Cad | 79% | internal pooled XM24 | | | SEQ ID NO: 183 | |
| IPD099-1Cae | 78.8% | WP_0603900299 | Aeromonas hydrophila | | SEQ ID NO: 184 | |
| IPD099-1Caf | 79% | XM27_pooled_NODE_19_3967 | | | SEQ ID NO: 185 | |
| IPD099-1Cag | 78.5% | XM26_pooled_NODE_315_83 | | | SEQ ID NO: 186 | |
| IPD099-1Cah | 78.8% | XM31_pooled_NODE_9686_1 | | | SEQ ID NO: 187 | |
| IPD099-1Cai | 79.3% | XM5_pooled_NODE_929_130 | | | SEQ ID NO: 188 | |
| IPD099-1Caj | 78.8% | AJQ55024 | | | SEQ ID NO: 189 | |
| IPD099-1Cak | 79% | WP_042064547 | | | SEQ ID NO: 190 | |
| IPD099-1Cal | 78.8% | WP_054544695 | | | SEQ ID NO: 191 | |
| IPD099-1Cam | 79.6% | partial_XM1_pooled_NODE_1535_6 | | | SEQ ID NO: 192 | |
| IPD099-1Can | 79.3% | WP_049047798 | | | SEQ ID NO: 193 | |
| IPD099-1Cao | 79% | WP_045789543 | | | SEQ ID NO: 194 | |
| IPD099-1Cap | 78.5% | XM30_pooled_NODE_8056_21 | | | SEQ ID NO: 195 | |
| IPD099-1Caq | 75.3% | JH77890-1 | | | SEQ ID NO: 196 | |
| IPD099-1Car | 79.3% | ANT67622 | Aeromonas hydrophila | | SEQ ID NO: 197 | |
| IPD099-1Cas | 79% | WP_065018003 | Aeromonas dhakensis | | SEQ ID NO: 198 | |
| IPD099-1Cat | 79% | PMCJ4115H2-1 | Aeromonas hydrophila | | SEQ ID NO: 199 | SEQ ID |
| | | | | | | NO: 600 |
| IPD099-1Cau | 78.5% | WP_073350653 | Aeromonas aquatica | | SEQ ID NO: 200 | |
| IPD099-1Cav | 78.5% | WP_076360968 | Aeromonas | | SEQ ID NO: 201 | |
| IPD099-1Ea | 50% | WP_043629758, | Chromobacterium piscinae | | SEQ ID NO: 202 | |
| | | WP_052247043 | | | | |
| IPD099-1Eb | 50% | WP_052247043 | | | SEQ ID NO: 203 | |
| IPD099-1Fa | 47% | NCBI EXIJ77038.1 | Erwinia amylovora | | SEQ ID NO: 204 | |
| IPD099-1Fb | 44% | internal strain JH78168-2; | Serratia liquefaciens | | SEQ ID NO: 205 | SEQ ID |
| | | JH20785-4 | | | | NO: 601 |
| IPD099-1Fc | 43.6% | WP_006320606 | Serratia plymuthica | | SEQ ID NO: 206 | |
| IPD099-1Fd | 45.8% | WP_044553510 | Serratia liquefaciens | | SEQ ID NO: 207 | |
| IPD099-1Fe | 43.9% | PMC3546H11-1 | Serratia marcescens | | SEQ ID NO: 208 | SEQ ID |
| | | | | | | NO: 602 |
| IPD099-1Ff | 45.3% | A0A0X2PAR3 | Serratia | | SEQ ID NO: 209 | |
| IPD099-1Fg | 44.2% | PMC3677F8-1 | Serratia ureilytica | | SEQ ID NO: 210 | |
| IPD099-1Fh | 41.6% | WP_069590127 | Salinivibrio sp. | | SEQ ID NO: 211 | |
| IPD099-1Fi | 45.5% | WP_073534092 | Serratia marcescens | | SEQ ID NO: 212 | |
| IPD099-1Fj | 43.9% | WP_074055057 | Serratia marcescens | | SEQ ID NO: 213 | |
| IPD099-1Ga | 34.6% | WP_063524174 | Vibrio sp. | | SEQ ID NO: 214 | |
| IPD099-1Gb | 35.4% | IB2016_0659 | | | SEQ ID NO: 215 | |
| IPD099-2Ab | 99% | JH82751-1 (1aa differ) | Aeromonas salmonicida | | SEQ ID NO: 216 | |
| IPD099-2Ac | 90% | JH71362-2, JH70211-1, | Aeromonas hydrophila, | WCRW | SEQ ID NO: 217 | |
| | | JH58748-2, JH77890-1, | Aeromonas salmonicida | | | |
| | | JH67338-1 (1aa), | | | | |
| | | JH77959-1 (1aa); internal | | | | |
| | | pooled KL1; NCBI | | | | |
| | | WP_005302109.1; | | | | |
| | | NCBI_WP_041216094 | | | | |
| | | (1aa) WP_045789542; | | | | |
| | | WP_049047797 | | | | |
| IPD099-2Ad | 90% | K01-14A1-1, K01-20A3-2, | Aeromonas hydrophila, | | SEQ ID NO: 218 | |
| | | NCBI WP_010633675.1, | Aeromonas dhakensis | | | |
| | | WP_017778853 (2aa); | | | | |
| | | WP_005302109 (1aa); | | | | |
| | | WP_042040644 (2aa); | | | | |
| | | internal pooled XM1 | | | | |
| IPD099-2Ae | 90% | NCBI WP_011706402.1, | Aeromonas hydrophila | | SEQ ID NO: 219 | |
| | | FW306009.1; AGM44514 | | | | |
| | | (1aa) | | | | |
| IPD099-2Af | 90% | NCBI EZH80102.1 | Aeromonas hydrophila | | SEQ ID NO: 220 | |
| IPD099-2Ag | 96% | | Aeromonas popoffii | | SEQ ID NO: 221 | |
| IPD099-2Ah | 99.7% | JH67351-1 | | | SEQ ID NO: 222 | |
| IPD099-2Ai | 99.7% | WP_042869733 | | | SEQ ID NO: 223 | |
| IPD099-2Aj | 99.7% | WP_043556154 | | | SEQ ID NO: 224 | |
| IPD099-2Ak | 99.7% | JH78147-1 | | | SEQ ID NO: 225 | |
| IPD099-2Al | 90% | WP_005302109 | | | SEQ ID NO: 226 | |
| IPD099-2Am | 93.9% | partial_XM1_pooled_NODE_6114_1 | | | SEQ ID NO: 227 | |
| IPD099-2An | 99.4% | WP_065403858 | Aeromonas piscicola | | SEQ ID NO: 228 | |
| IPD099-2Ao | 90.3% | WP_073350654 | Aeromonas aquatica | | SEQ ID NO: 229 | |
| IPD099-2Ba | 89.7% | JH67338-1 | | | SEQ ID NO: 230 | SEQ ID |
| | | | | | | NO: 603 |
| IPD099-2Bb | 89.7% | WP_041216094 | | | SEQ ID NO: 231 | |
| IPD099-2Bc | 89.4% | KL1_pooled_NODE_194_599 | | | SEQ ID NO: 232 | |
| IPD099-2Bd | 89.4% | WP_045789542 | | | SEQ ID NO: 233 | |
| IPD099-2Be | 89.7% | WP_048047797 | | | SEQ ID NO: 234 | |
| IPD099-2Bf | 89.4% | WP_060390298 | | | SEQ ID NO: 235 | |
| IPD099-2Bg | 89.7% | WP_062826546 | | | SEQ ID NO: 236 | |
| IPD099-2Bh | 89.7% | WP_017778853 | | | SEQ ID NO: 237 | |
| IPD099-2Bi | 89.7% | WP_042040644 | | | SEQ ID NO: 238 | |
| IPD099-2Bj | 86.1% | WP_075384207 | Aeromonas hydrophila | | SEQ ID NO: 239 | |
| IPD099-2Ca | 77% | NCBI EXIJ77039.1 | Erwinia amylovora | | SEQ ID NO: 240 | |
| IPD099-2Cb | 76.5% | WP_043629747 | Chromobacterium piscinae | | SEQ ID NO: 241 | |
| IPD099-2Cc | 77.4% | WP_069590129 | Salinivibrio sp. | | SEQ ID NO: 242 | |
| IPD099-2Cd | 75.9% | WP_071109676 | Chromobacterium amazonense | | SEQ ID NO: 243 | |
| IPD099-2Da | 62% | SSP605C7, SSP605C7-1n | Serratia plymuthica | | SEQ ID NO: 244 | SEQ ID |
| | | | | | | NO: 604 |
| IPD099-2Db | 62% | NCBI WP_010644719.1, | Vibrio campbellii | | SEQ ID NO: 245 | |
| | | WP_005532945.1 | | | | |
| IPD099-2Dc | 61% | JH20785-4 | Serratia plymuthica | | SEQ ID NO: 246 | |
| IPD099-2Dd | 60% | NCBI WP_006320605.1 | Serratia plymuthica | | SEQ ID NO: 247 | |
| IPD099-2De | 61.3% | WP_005532945 | Vibrio campbellii | | SEQ ID NO: 248 | |
| IPD099-2Df | 62.3% | PMC3546H11-1 | | | SEQ ID NO: 249 | |
| IPD099-2Dg | 63% | WP_063524175 | Vibro sp. | | SEQ ID NO: 250 | |
| IPD099-2Dh | 61.7% | PMC3677FB-1 | Serratia ureilytica | | SEQ ID NO: 251 | |
| IPD099-2Di | 62% | WP_074055056 | Serratia marcescens | | SEQ ID NO: 252 | |
| IPD099-2Di | 61.2% | WP_073534094 | Serratia marcescens | | SEQ ID NO: 253 | |
| IPD099-2Ea | 59% | GAK2831.1 | Serratia | | SEQ ID NO: 254 | |
| | | | liquefaciens | | | |
| IPD099-2Eb | 59.5% | A0A0X2PCH1 | Serratia | | SEQ ID NO: 255 | |
| IPD099-2Fa | 41.1% | WP_035095894, | Aquimarina megaterium | | SEQ ID NO: 256 | |
| | | WP_035095894 | | | | |
| IPD099-2Fb | 46.9% | WP_035827333, | Janthinobacterium sp. | | SEQ ID NO: 257 | |
| | | WP_051958568 | | | | |
| IPD099-2Fc | 44.5% | WP_045872306 | Tolypothrix sp. | | SEQ ID NO: 258 | |
| IPD099-2Fd | 40.1% | WP_011221504 | Photobacterium | | SEQ ID NO: 259 | |
| | | | profundum | | | |
| IPD099-2Fe | 46.5% | WP_051990888 | Janthinobacterium | | SEQ ID NO: 260 | |
| | | | lividum | | | |
| IPD099-2Ff | 46.8% | WP_058048092 | Janthinobacterium sp. | | SEQ ID NO: 261 | |
| IPD099-2Fg | 42.6% | A0A0Q4VRG6 | Rhizobium sp. | | SEQ ID NO: 262 | |
| IPD099-2Fh | 41% | WP_035095894 | | | SEQ ID NO: 263 | |
| IPD099-2Fi | 46.5% | WP_051958568 | | | SEQ ID NO: 264 | |
| IPD099-2Fj | 44.6% | A0A137SAT8 | Moritella sp | | SEQ ID NO: 265 | |
| IPD099-2Fk | 46.8% | WP_070302362 | Janthinobacterium sp. | | SEQ ID NO: 266 | |
| IPD099-2Fl | 46.8% | WP_072453575 | Janthinobacterium lividum | | SEQ ID NO: 267 | |
| IPD099-2Ga | 33% | AMG67705 | Providencia stuartii | | SEQ ID NO: 268 | |
| IPD099-2Gb | 38.6% | A0A0T9L222 | Yersinia nurmii | | SEQ ID NO: 269 | |
| IPD099-2Gc | 36.9% | WP_072082496 | Yersinia kristensenii | | SEQ ID NO: 270 | |
| IPD099-2Gd | 30.2% | WP_074407284 | Aquimarina megaterium | | SEQ ID NO: 271 | |
| IPD099-3Ab | 99% | JH82751-1 (1aa differ from | Aeromonas salmonicida | WCRW | SEQ ID NO: 272 | SEQ ID |
| | | Aa); JH78490-2 (1aa); | | | | NO: 605 |
| | | JH91676-2 | | | | |
| IPD099-3Ac | 99% | JH673511 (2aa differ from Aa) | Aeromonas | WCRW | SEQ ID NO: 273 | SEQ ID |
| | | | | | | NO: 606 |
| IPD099-3Ad | 99% | JH67338-1; JH77959-1; | Aeromonas | WCRW | SEQ ID NO: 274 | SEQ ID |
| | | JH77890-1 (1aa); | | | | NO: 607 |
| | | JH78710-1; NCBI - | | | | |
| | | WP_042064546 (1aa); | | | | |
| | | WP_044800223 | | | | |
| IPD099-3Ae | 99% | JH58748-2 | Aeromonas | WCRW | SEQ ID NO: 275 | SEQ ID |
| | | | | | | NO: 608 |
| IPD099-3Af | 96% | NCBI KFN17898.1 | Aeromonas salmonicida | | SEQ ID NO: 276 | |
| IPD099-3Ag | 96% | NCBI WP_011706403.1; | Aeromonas hydrophila | | SEQ ID NO: 277 | |
| | | JH99577-2 | | | | |
| IPD099-3Ah | 96% | NCBI WP_024944912.1; | Aeromonas hydrophila | | SEQ ID NO: 278 | |
| | | internal- JH91484-1 | | | | |
| IPD099-3Ai | 96% | NCBI WP_029300414.1; | Aeromonas hydrophila | | SEQ ID NO: 279 | |
| | | WP_045527026; | | | | |
| | | WP_049047795 | | | | |
| IPD099-3Aj | 96% | NCBI WP_016351062.1 | Aeromonas hydrophila | | SEQ ID NO: 280 | |
| IPD099-3Ak | 96% | NCBI WP_029302782.1 | Aeromonas hydrophila | | SEQ ID NO: 281 | |
| | | | | | SEQ ID NO: 282 | |
| IPD099-3Al | 96% | NCBI WP_019840112.1 | Aeromonas | | | |
| IPD099-3Am | 96% | NCBI KER63313.1 | Aeromonas hydrophila | | SEQ ID NO: 283 | |
| IPD099-3An | 96% | NCBI EZH80103.1 | Aeromonas hydrophila | | SEQ ID NO: 284 | |
| IPD099-3Ao | 95% | JH71362-2, JH70211-1 | Aeromonas | WCRW | SEQ ID NO: 285 | SEQ ID |
| | | | | | | NO: 609 |
| IPD099-3Ap | 95% | K01-14A1-1, K01-20A3-2 | Aeromonas | | SEQ ID NO: 286 | |
| IPD099-3Aq | 95% | NCBI WP_017782951.1 | Aeromonas hydrophila | | SEQ ID NO: 287 | |
| IPD099-3Ar | 95% | NCBI WP_017778852.1 | Aeromonas hydrophila | | SEQ ID NO: 288 | |
| IPD099-3As | 95% | NCBI WP_017764244.1 | Aeromonas hydrophila | | SEQ ID NO: 289 | |
| IPD099-3Ar | 95% | NCBI WP_017784070.1 | Aeromonas hydrophila | | SEQ ID NO: 290 | |
| IPD099-3Au | 95% | NCBI WP_005302111.1 | Aermonas | | SEQ ID NO: 291 | |
| IPD099-3Av | 95% | NCBI WP_017786781.1 | Aeromonas hydrophila | | SEQ ID NO: 292 | |
| IPD099-3Aw | 95% | NCBI WP_010633674.1 | Aeromonas dhakensis | | SEQ ID NO: 293 | |
| IPD099-3Ax | 94% | NCBI WP_024941131.1 | Aeromonas hydrophila | | SEQ ID NO: 294 | |
| IPD099-3Ay | 96% | WP_041216095 | Aeromonas hydrophila | | SEQ ID NO: 295 | |
| IPD099-3Az | 97.7% | WP_042869731 | Aeromonas piscicola | | SEQ ID NO: 296 | |
| IPD099-3Aaa | 99.2% | WP_043556156 | Aeromonas bestiarum | | SEQ ID NO: 297 | |
| IPD099-3Aab | 94.7% | A0A0A5L9U8_{—} | Aeromonas hydrophila | | SEQ ID NO: 298 | |
| IPD099-3Aac | 98.1% | JH78147-1 | Haemophilus piscium | | SEQ ID NO: 299 | |
| IPD099-3Aad | 99.2% | JH78490-2 | | | SEQ ID NO: 300 | |
| IPD099-3Aae | 99.2% | JH91676-2 | | | SEQ ID NO: 301 | |
| IPD099-3Aad | 96.2% | JH77890-1 | | | SEQ ID NO: 302 | |
| IPD099-3Aag | 96.2% | WP_042064546 | | | SEQ ID NO: 303 | |
| IPD099-3Aah | 96.6% | JH78710-1 | | | SEQ ID NO: 304 | |
| IPD099-3Aai | 96.2% | WP_044800223 | | | SEQ ID NO: 305 | |
| IPD099-3Aaj | 96.2% | XM24_pooled_NODE_35_464 | | | SEQ ID NO: 306 | |
| IPD099-3Aak | 96.2% | JH89577-2 | | | SEQ ID NO: 307 | |
| IPD099-3Aal | 96.6% | JH91484 | | | SEQ ID NO: 308 | |
| IPD099-3Aam | 97% | WP_060390297 | | | SEQ ID NO: 309 | |
| IPD099-3Aan | 97% | WP_045527026 | | | SEQ ID NO: 310 | |
| IPD099-3Aao | 96.2% | WP_049047796 | | | SEQ ID NO: 311 | |
| IPD099-3Aap | 96.6% | XM27_pooled_NODE_19_5114 | | | SEQ ID NO: 312 | |
| IPD099-3Aaq | 96.2% | ANT67620 | Aeromonas hydrophila | | SEQ ID NO: 313 | |
| IPD099-3Aar | 97.7% | WP_065403859 | Aeromonas piscicola | | SEQ ID NO: 314 | |
| | | | | | SEQ ID NO: 315 | |
| IPD099-3Aas | 95.1% | WP_065018002 | Aeromonas dhakensis | | | |
| IPD099-3Ca | 77.8% | WP_042036785 | Aeromonas popoffii | | SEQ ID NO: 316 | |
| IPD099-3Ea | 52% | NCBI EXIJ77040.1 | Erwinia mallotivora | | SEQ ID NO: 317 | |
| IPD099-3Fa | 47% | NCBI WP_006532948.1; | Vibrio campbellii | | SEQ ID NO: 318 | |
| | | WP_010644721.1 | | | | |
| IPD099-3Fb | 46% | NCBI GAK28232.1 | Serratia liquefaciens | | SEQ ID NO: 319 | |
| IPD099-3Fc | 42% | JH20785-4, JH78168-2 | Serratia plymuthica | WCRW | SEQ ID NO: 320 | SEQ ID |
| | | | | | | NO: 610 |
| IPD099-3Fd | 46.3% | WP_010644721 | Vibrio campbellii | | SEQ ID NO: 321 | |
| IPD099-3Fe | 49% | WP_043629750 | Chromobacterium | | SEQ ID NO: 322 | |
| | | | piscinae | | | |
| IPD099-3Ff | 43.8% | PMC3546H11-1 | Serratia marcescens | | SEQ ID NO: 323 | |
| IPD099-3Fg | 44.2% | A0A0X2PC11 | Serratia | | SEQ ID NO: 324 | |
| IPD099-3Fh | 45.6% | WP_063524176 | Vibrio sp. | | SEQ ID NO: 325 | |
| IPD099-3Fi | 43.8% | PMC3677F8-1 | Serratia ureilytica | | SEQ ID NO: 326 | |
| IPD099-3Fi | 48.5% | WP_069590131 | Salinivibrio sp. | | SEQ ID NO: 327 | |
| IPD099-3Fk | 47.4% | WP_071109675 | Chromobacterium | | SEQ ID NO: 328 | |
| | | | amazonense | | | |
| IPD099-3Fl | 43.4% | WP_074055055 | Serratia marcescens | | SEQ ID NO: 329 | |
| IPD099-3Fm | 43.4% | WP_073534095 | Serratia marcescens | | SEQ ID NO: 330 | |
| IPD099-3Ga | 37.8% | ANS44859 | Serratia | | SEQ ID NO: 331 | |
| | | | plymuthica | | | |

Table 10 shows the IPD100-1Aa and IPD100-2Aa polypeptide homologs identfied, sequence identification numbers for each and the bacterial isolates they were identified from.

TABLE 10

| | Identity to | | | | | |
|---|---|---|---|---|---|---|
| | IPD100- | | | | | |
| IPD Name | 1/2Aa | Source | Species | activity | Protein | DNA |
| IPD100-1Aa | | JH55673-1 JH19870-2, | Pseudomonas gessardii | WCRW | SEQ ID NO: 332 | SEQ ID NO: 611 |
| | | JH81777-2, JH81870-2 | | | | |
| IPD100-2Aa | | JH55673-1, JH81777-2, | Pseudomonas gessardii | | SEQ ID NO: 333 | SEQ ID NO: 612 |
| | | JH81870-2 | | | | |
| IPD100-1Ba | 86% | JGI 2036107561 | mountain pine beetle microbes | | SEQ ID NO: 334 | |
| IPD100-1Ea | 59.9% | WP_060293698 | Burkholderia ubonensis | | SEQ ID NO: 335 | |
| IPD100-1Fa | 49% | NCBI WP_034419751.1 | Candidatus Entotheonella sp. | | SEQ ID NO: 336 | |
| IPD100-2Ab | 99% | JH19870-2 | Pseudomonas gessardii | WCRW | SEQ ID NO: 337 | SEQ ID NO: 613 |
| IPD100-2Ba | 84.8% | internal pooled XM17 | | | SEQ ID NO: 338 | |
| IPD100-2Ca | 72.3% | WP_060293699 | Burkholderia ubonensis | | SEQ ID NO: 339 | |
| IPD100-2Ea | 56% | NCBI ETW97596.1 | Candidatus Entotheonella sp. | | SEQ ID NO: 340 | |
| IPD100-2Ga | 36.8% | A0A0P9N9S1 | Pseudomonas syringae | | SEQ ID NO: 341 | |
| IPD100-2Gb | 36.2% | A0A0Q6U9X6 | Duganella sp. | | SEQ ID NO: 342 | |
| IPD100-2Gc | 36.8% | A0A0P9THA2 | | | SEQ ID NO: 343 | |
| IPD100-2Gd | 34.3% | A0A0Q8R323 | | | SEQ ID NO: 344 | |
| IPD100-2Ge | 34.5% | WP_065336555 | Salmonella enterica | | SEQ ID NO: 345 | |
| IPD100-2Gf | 32.7% | WP_064966240 | Tenacibaculum ovolyticum | | SEQ ID NO: 346 | |
| IPD100-2Gg | 33.5% | A0A0B2TSA4 | Dickeya solani | | SEQ ID NO: 347 | |
| IPD100-2Gh | 35% | WP_074607035 | Pelobacter steyni | | SEQ ID NO: 348 | |
| IPD100-2Gi | 33.8% | WP_076246106 | Mycobacterium sp. | | SEQ ID NO: 349 | |

Table 11 shows the IPD105Aa polypeptide homologs identified, sequence identification numbers for each and the bacterial isolates they were identified from.

TABLE 11

| | Identity to | | | | | |
|---|---|---|---|---|---|---|
| IPD Name | IPD105Aa | Source | Species | activity | Protein | DNA |
| IPD105Aa | | JH90961-1, JH97541-1, | Chromobacterium aquaticum | WCRW | SEQ ID NO: 350 | SEQ ID NO: 614 |
| | | JH90377-1, SSP555D9c, | | | | |
| IPD105Ab | 99% | NCBI WP_019104214. | Chromobacterium | WCRW | SEQ ID NO: 351 | SEQ ID NO: 615 |
| | | | sp. C-61 | | | |
| IPD105Ac | 99% | JH96390-2, JH94105-2, | | | SEQ ID NO: 362 | |
| | | JH97517-1, JH97285-1, | | | | |
| | | JH97240-1, JH90688-1, | | | | |
| | | NCBI_WP_043589636.1 | | | | |
| IPD105Ad | 98% | JH90976-1 | | | SEQ ID NO: 353 | |
| IPD105Ae | 99% | NCBI WP_043637141.1 | Chromobacterium | | SEQ ID NO: 354 | |
| | | | haemolyticum | | | |
| IPD105Af | 95.1% | XM27_pooled | | | SEQ ID NO: 355 | |
| IPD105Ba | 89.6% | WP_048410860 | Chromobacterium | | SEQ ID NO: 356 | |
| IPD105Ea | 56% | JH94452-1 | Chromobacterium | WCRW | SEQ ID NO: 357 | SEQ ID NO: 616 |
| IPD105Eb | 55.7% | NCBI WP_045052236.1, | Chromobacterium | | SEQ ID NO: 358 | |
| | | gb\|KJH65942.1, | violaceum | | | |
| | | WP_011134423.1 | | | | |
| IPD105Ec | 58% | NCBI WP_021475143.1, | Pseudogulbenkiania | | SEQ ID NO: 359 | |
| | | gb\|ERE20223.1 | ferrooxidans | | | |
| IPD105Ed | 51% | NCBI WP_043621092.1, | Chromobacterium | | SEQ ID NO: 360 | |
| | | gb\|KIA81985.1 | piscinae | | | |
| IPD105Ee | 56.6% | WP_052258131, | Chromobacterium subtsugae | | SEQ ID NO: 361 | |
| | | WP_052941274 | | | | |
| IPD105Ef | 51.2% | A0A0J6QEL9 | | | SEQ ID NO: 362 | |
| IPD105Eg | 56% | WP_052941274 | | | SEQ ID NO: 363 | |
| IPD105Eh | 53.7% | WP_071111093 | Chromobacterium | | SEQ ID NO: 364 | |
| IPD105Fa | 49.8% | WP_07110944.2 | Chromobacterium amazonense | | SEQ ID NO: 365 | |

Table 12 shows a matrix table of pair-wise identity relationships for global alignments of the IPD105Aa homologs, based upon the Needleman-Wunsch algorithm, as implemented in the Needle program (EMBOSS tool suite).

TABLE 12

| | IPD105Ab | SPD105Ac | IPD105Ad | IPD105Ae | IPD105Af | IPD105Ba | IPD105Ea |
|---|---|---|---|---|---|---|---|
| IPD105Aa | 93.9 | 98.8 | 98.2 | 93.3 | 95.1 | 89.6 | 52.4 |
| IPD105Ab | — | 93.3 | 92.7 | 98.1 | 90.2 | 94.2 | 55.8 |
| IPD105Ac | — | — | 99.4 | 92.7 | 96.3 | 89.0 | 52.4 |
| IPD105Ad | — | — | — | 92.1 | 95.7 | 88.4 | 52.4 |
| IPD105Ae | — | — | — | — | 90.9 | 94.8 | 54.5 |
| IPD105Af | — | — | — | — | — | 92.1 | 51.8 |
| IPD105Ba | — | — | — | — | — | — | 54.5 |
| IPD105Ea | — | — | — | — | — | — | — |
| IPD105Eb | — | — | — | — | — | — | — |
| IPD105Ec | — | — | — | — | — | — | — |
| IPD105Ed | — | — | — | — | — | — | — |
| IPD105Ee | — | — | — | — | — | — | — |
| IPD105Ef | — | — | — | — | — | — | — |
| IPD105Eg | — | — | — | — | — | — | — |

| | IPD105Eb | IPD105Ec | IPD105Ed | IPD105Ee | IPD105Ef | IPD105Eg | IPD105Eh |
|---|---|---|---|---|---|---|---|
| IPD105Aa | 53.0 | 53.0 | 48.8 | 46.5 | 53.0 | 56.0 | 65.1 |
| IPD105Ab | 56.4 | 56.4 | 51.0 | 59.7 | 56.4 | 59.1 | 58.2 |
| IPD105Ac | 53.0 | 53.0 | 48.2 | 57.2 | 53.0 | 56.6 | 55.8 |
| IPD105Ad | 52.4 | 53.0 | 48.2 | 57.2 | 52.4 | 56.6 | 55.8 |
| IPD105Ae | 55.1 | 55.1 | 49.7 | 59.1 | 55.1 | 58.5 | 57.0 |
| IPD105Af | 52.4 | 52.4 | 47.6 | 58.6 | 52.4 | 56.0 | 55.2 |
| IPD105Ba | 55.1 | 55.1 | 50.3 | 57.2 | 55.1 | 56.6 | 56.2 |
| IPD105Ea | 92.5 | 90.5 | 59.0 | 65.4 | 93.2 | 64.7 | 67.3 |
| IPD105Eb | — | 91.8 | 59.6 | 66.7 | 99.3 | 66.0 | 67.9 |
| IPD105Ec | — | — | 58.3 | 65.4 | 92.6 | 64.7 | 66.7 |
| IPD105Ed | — | — | — | 60.9 | 59.6 | 60.3 | 61.4 |
| IPD105Ee | — | — | — | — | 66.7 | 99.4 | 88.5 |
| IPD105Ef | — | — | — | — | — | 66.0 | 67.9 |
| IPD105Eg | — | — | — | — | — | — | 87.8 |

Table 13 shows the IPD106-1Aa and IPD106-2Aa polypeptide homologs identified, sequence identification numbers for each and the bacterial isolates they were identified from.

TABLE 13

| | Identity to | | | | | |
|---|---|---|---|---|---|---|
| | IPD106- | | | | | |
| IPD Name | 1/2Aa | Source | Species | activity | | |
| IPD106-1Aa | | JH8820-1 | Chitinophaga | WCRW | SEQ ID NO: 366 | SEQ ID NO: 617 |
| | | | pinensis | | | |
| IPD106-2Aa | | JH48820-1 | Chitinophaga | | SEQ ID NO: 367 | SEQ ID NO: 618 |
| | | | pinensis | | | |
| IPD106-1Ab | 92.6% | internal- D6270037 | Taxon P0045 pooled | | SEQ ID NO: 368 | |
| IPD106-1Ca | 72.8% | PMCJ3307D3-1 | Chitinophaga sp. | WCRW | SEQ ID NO: 369 | SEQ ID NO: 619 |
| IPD106-1Da | 63% | JGI: 2165210246 | Switchgrass rhizosphere metagenome | | SEQ ID NO: 370 | |
| IPD106-1Ea | 51.3% | WP_071503955 | Arsenicibacter rosenii | | SEQ ID NO: 371 | |
| IPD106-2Ba | 87.3% | internal- D6270037 | Taxon P0045 pooled | WCRW | SEQ ID NO: 372 | SEQ ID NO: 620 |
| IPD106-2Bb | 80.2% | PMCJ3307D3-1 | Chitinophaga sp. | | SEQ ID NO: 373 | |
| IPD106-2Ea | 55% | JGI: 2165039070 | Switchgrass rhizosphere | | SEQ ID NO: 374 | |
| | | | metagenome | | | |
| IPD106-2Fa | 41.4% | WP_071503954 | Arsenicibacter rosenii | | SEQ ID NO: 375 | |
| IPD106-2Ga | 37.6% | A0A0G3A128 | Archangium gephyra | | SEQ ID NO: 376 | |

Table 14 shows the IPD107Aa polypeptide homoologs identified, sequence identification numbers for each and the bacterial isolates they were identified from.

TABLE 14

| | Identity to | | | | | |
|---|---|---|---|---|---|---|
| IPD Name | IPD107Aa | Source | Species | activity | Protein | DNA |
| IPD107Aa | | JH60888-1, JH62342-2, | Pseudomonas brassicacearum | WCRW | SEQ ID NO: 377 | SEQ ID NO: 621 |
| | | JH61870-1, JH61310-1-1, | | | | |
| | | JH60848-1; WP_040071980 | | | | |
| | | (2aa) | | | | |
| IPD107Ab | 96.3% | JH77717-1, JH77706-1, | Pseudomonas brassicacearum | | SEQ ID NO: 378 | SEQ ID NO: 622 |
| | | JH75881-1, SSP347B8a | | | | |
| | | (1aa) | | | | |
| IPD107Ac | 92.5% | WP_017901970 | Pseudomonas | | SEQ ID NO: 379 | |
| | | | fuscovaginae | | | |
| IPD107Ad | 98.1% | WP_040071980 | | | SEQ ID NO: 380 | |
| IPD107Ae | 95.3% | SSP347B8a | | | SEQ ID NO: 381 | |
| IPD107Ba | 83.2% | JH93481-1 | Pseudomonas parafulva | | SEQ ID NO: 362 | SEQ ID NO: 623 |
| IPD107Bb | 80.4% | SSP10A9a, SSP509A5b, | | | SEQ ID NO: 363 | |
| | | SSP509A5a; | | | | |
| | | WP_017124730 | | | | |
| IPD107Ca | 77.6% | SSP652H10-1, SSP652H3-1, | Pseudomonas | | SEQ ID NO: 364 | SEQ ID NO: 624 |
| | | SSP652H2-1, SSP650H5-1, | chlororaphis | | | |
| | | SSP650H1-2, SSP648H11-1, | | | | |
| | | SSP648H7-1, SSP648H5-1 | | | | |
| IPD107Cc | 76.6% | LBNL154D2-1, LBNL154C2-1 | | | SEQ ID NO: 365 | |
| | | LBNL154B2-1, | | | | |
| | | LBNL151A2-2, LBNL151D1-1 | | | | |
| | | LBNL151B1-2, | | | | |
| | | SSP630B11-1, | | | | |
| | | SSP596B11d, LBNL151C1-1 | | | | |
| | | SSP631G11-1, | | | | |
| | | SSP630H8-1, SSP596B7c, | | | | |
| | | JH23300-1, JH68784-1, | | | | |
| | | JH67396-1, MX3C7, | | | | |
| | | LBV08710-7, LBV10044-5, | | | | |
| | | SSP374H3-7, LBV8556-5, | | | | |
| | | LBV10056, JH22647-2, | | | | |
| | | JH22857-2, JH22817-1, | | | | |
| | | JH59295-2, JH58797-1, | | | | |
| | | JH58576-1, JH23965-2, | | | | |
| | | JH23148-1, JH22841-2, | | | | |
| | | JH21457-2 JH21296-2, | | | | |
| | | JH20150-1, BAQ71928, | | | | |
| | | A0A0D689V9, | | | | |
| | | WP_016963092, JH20286-4 | | | | |
| | | (1aa) | | | | |
| IPD107Cd | 73.8% | JH71892-1, JH71833-2, | Pseudomonas protegens | | SEQ ID NO: 386 | |
| | | JH71826-1, JH71539-1 | | | | |
| IPD107Ce | 74.8% | JH72069-2, JH1668-2, | Pseudomonas monteilii | | SEQ ID NO: 387 | |
| | | JH69828-1 (1aa) | | | | |
| IPD107Cf | 71% | SSP587D6-1 | | | SEQ ID NO: 388 | |
| IPD107Cg | 74.1% | JH85760-1 | Pseudomonas | | SEQ ID NO: 389 | |
| | | | frederiksbergensis | | | |
| IPD107Ch | 71% | SSP459A9-4, SSP551G4b | | | SEQ ID NO: 390 | |
| | | (1aa), SSP551G5a (1aa) | | | | |
| IPD107Ci | 70.4% | JH18994-3, JH18447-2 | Pseudomonas rhodesiae | | SEQ ID NO: 391 | |
| IPD107Cj | 70.4% | SSP719C3-1, SSP616E3-1, | | | SEQ ID NO: 392 | SEQ ID NO: 625 |
| | | SSP615D3-2, SSP616E6-2, | | | | |
| | | LBV4325, JH69839-1; | | | | |
| | | WP_007952780; SSP8B7 | | | | |
| | | (1aa), JH19281-2 (1aa), | | | | |
| | | J3FC90, WP_003220553 | | | | |
| | | (1aa), WP_034151843 (2aa) | | | | |
| IPD107Ck | 77.6% | WP_023969973, SSP652H6-1 | Pseudomonas | | SEQ ID NO: 393 | |
| | | (1aa), SSP652H1-2 (1aa), | chlororaphis | | | |
| | | SSP652D6-1 (1aa), | | | | |
| | | SSP650H4-1 (1aa), | | | | |
| | | SSP143C2 (1aa) | | | | |
| IPD107Cl | 74.8% | WP_025128520 | Pseudomonas sp. | | SEQ ID NO: 394 | |
| IPD107Cm | 71.3% | WP_038978461 | Pseudomonas | | SEQ ID NO: 395 | |
| IPD107Cn | 71.3% | JH102832-1 | Pseudomonas | | SEQ ID NO: 396 | |
| | | | fluorescens | | | |
| IPD107Co | 71.3% | internal pooled XM17 | | | SEQ ID NO: 397 | |
| IPD107Cp | 71.3% | internal pooled XM17 | | | SEQ ID NO: 398 | |
| IPD107Cq | 70.4% | WP_054616563 | Pseudomonas sp. | | SEQ ID NO: 399 | |
| IPD107Ct | 74.1% | internal pooled XM24 | | | SEQ ID NO: 400 | |
| IPD107Cu | 70.4% | internal pooled XM24 | | | SEQ ID NO: 401 | |
| IPD107Cv | 71.3% | P0114 | Taxon pooled sample | | SEQ ID NO: 402 | |
| IPD107Cx | 77.6% | JH20286-4 | | | SEQ ID NO: 403 | |
| IPD107Cy | 73.8% | XM31_pooled_NODE_102_2137 | | | SEQ ID NO: 404 | |
| IPD107Cz | 73.8% | JH69828-1 | | | SEQ ID NO: 405 | |
| IPD107Caa | 73.1% | WP_054047873 | | | SEQ ID NO: 406 | |
| IPD107Cab | 70.1% | SSP551G4b | | | SEQ ID NO: 407 | |
| IPD107Cac | 70.4% | WP_034151843 | | | SEQ ID NO: 408 | |
| IPD107Cad | 70.4% | XM22_pooled_NODE_3714_78 | | | SEQ ID NO: 409 | |
| IPD107Cae | 76.6% | SSP652H6-1 | | | SEQ ID NO: 410 | |
| IPD107Caf | 70.4% | XM17_pooled_NODE_17832_14 | | | SEQ ID NO: 411 | |
| IPD107Cag | 72.2% | PMC3015E8-2 | | | SEQ ID NO: 412 | |
| IPD107Cah | 71.3% | PMC3618B11-1 | | | SEQ ID NO: 413 | |
| IPD107Cai | 70.4% | internal pooled HK-23 | | | SEQ ID NO: 414 | |
| IPD107Caj | 70.4% | internal pooled HK-24 | | | SEQ ID NO: 415 | |
| IPD107Cak | 75.7% | WP_068580103 | Pseudomonas | | SEQ ID NO: 416 | |
| IPD107Da | 68.5% | SSP452E2-1 | Pseudomonas | | SEQ ID NO: 417 | SEQ ID NO: 626 |
| | | | brassicacearum | | | |
| IPD107Db | 68.5% | SSP562B7b, JH58586-2 | | | SEQ ID NO: 418 | |
| | | (1aa), JH31096-2 (1aa), | | | | |
| | | JH34637-1 (1aa), JH32046-2 | | | | |
| | | (1aa), JH17095-4 (1aa), | | | | |
| | | JH17069-1 (1aa), | | | | |
| | | SSP346B1a (2aa), JH18316-4 | | | | |
| | | (2aa), JH31283-1 (2aa), | | | | |
| | | JH17340-4 (2aa), JH17338-2 | | | | |
| | | (2aa) | | | | |
| IPD107Dc | 69.2% | JH19896-4, JH19820-2, | Pseudomonas | | SEQ ID NO: 419 | |
| | | JH90148-1 (1aa), JH89756-1 | chlororaphis | | | |
| | | (1aa), JH94168-1 (1aa), | | | | |
| | | JH94122-1 (1aa), JH94108-1 | | | | |
| | | (1aa), JH94092-2 (1aa), | | | | |
| | | JH94070-1 (1aa), JH94046-1 | | | | |
| | | (1aa), JH94031-1 (1aa), | | | | |
| | | JH72002-1 (1aa), JH71363-1 | | | | |
| | | (1aa), JH66281-1 (1aa), | | | | |
| | | SSN45H3 (1aa), JH36960-1 | | | | |
| | | (1aa), SSP560H5b (2aa), | | | | |
| | | DC14B2 (2aa) | | | | |
| IPD107Dd | 68.5% | JH31121-1 | Pseudomonas | | SEQ ID NO: 420 | |
| | | | frederiksbergensis | | | |
| IPD107De | 69.4% | JH72599-2, SSP475F12-1, | Pseudomonas | | SEQ ID NO: 421 | |
| | | WP_039764506 | fluorescens | | | |
| IPD107Df | 68.5% | JH87747-2, JH75493-1 | Serratia plymuthica | | SEQ ID NO: 422 | |
| IPD107Dg | 69.4% | WP_02511024, | Pseudomonas sp. | | SEQ ID NO: 423 | |
| | | WP_007919145 (1aa), | | | | |
| | | WP_042557100 (1aa) | | | | |
| IPD107Dh | 68.5% | WP_007963451 | Pseudomonas sp. | | SEQ ID NO: 424 | |
| IPD107Di | 67.6% | WP_047601000 | Pseudomonas sp. | | SEQ ID NO: 425 | |
| IPD107Dj | 69.4% | PMC3508D8-1 | Pseudomonas | | SEQ ID NO: 426 | |
| | | | fluorescens | | | |
| IPD107Dk | 67.6% | internal pooled XM17 | | | SEQ ID NO: 427 | |
| IPD107Dl | 68.5% | internal pooled XM20 | | | SEQ ID NO: 428 | |
| IPD107Dm | 68.5% | JH31096-2 | | | SEQ ID NO: 429 | |
| IPD107Dn | 69.4% | SSP346B1a | | | SEQ ID NO: 430 | |
| IPD107Do | 69.4% | JH58586-2 | | | SEQ ID NO: 431 | |
| IPD107Dp | 68.5% | JH31283-1 | | | SEQ ID NO: 432 | |
| IPD107Dq | 69.2% | JH90148-1 | | | SEQ ID NO: 433 | |
| IPD107Dr | 68.2% | SSP560H5b | | | SEQ ID NO: 434 | |
| IPD107Ds | 69.2% | DC14B2 | | | SEQ ID NO: 435 | |
| IPD107Dt | 67.6% | WP_007919145 | | | SEQ ID NO: 436 | |
| IPD107Du | 67.6% | WP_042557100 | | | SEQ ID NO: 437 | |
| IPD107Dv | 69.4% | XM24_pooled_NODE_138_1840 | | | SEQ ID NO: 438 | |
| IPD107Dw | 68.5% | XM17_pooled_NODE_2233_214 | | | SEQ ID NO: 439 | |
| IPD107Dx | 69.4% | SSP8B7 | | | SEQ ID NO: 440 | |
| IPD107Dy | 69.4% | WP_003220553 | | | SEQ ID NO: 441 | |
| IPD107Dz | 69.4% | XM16_pooled_NODE_40846_1 | | | SEQ ID NO: 442 | |
| IPD107Daa | 68.5% | PMCJ3261G6-1 | | | SEQ ID NO: 443 | |
| IPD107Dab | 69.4% | WP_064117478 | Pseudomonas | | SEQ ID NO: 444 | |
| | | | fluorescens | | | |
| IPD107Dac | 66.7% | SDR78243 | Pseudomonas sp. | | SEQ ID NO: 445 | |
| IPD107Fa | 42.7% | JH91906-1, JH91791-1 | Chromobacterium haemolyticum | | SEQ ID NO: 446 | SEQ ID NO: 627 |
| IPD107Fb | 41.7% | WP_02834481 | Bradyrhizobium elkanii | | SEQ ID NO: 447 | |
| IPD107Fc | 42.6% | WP_029064374 | Bradyrhizobium sp. | | SEQ ID NO: 448 | SEQ ID NO: 628 |
| IPD107Fd | 40.7% | WP_038361133 | Bradyrhizobium elkanii | | SEQ ID NO: 449 | |
| IPD107Fe | 45.4% | WP_050632090 | Bradyrhizobium sp. | | SEQ ID NO: 450 | |
| IPD107Ff | 42.1% | Taxon Pooled | | | SEQ ID NO: 451 | |
| | | P0053_D6840010_NODE_331_956 | | | | |
| IPD107Ga | 33.3% | XM28_pooled_NODE_163_467 | | | SEQ ID NO: 452 | |

Table 15 shows the IPD111Aa polypeptide homologs identified, sequence identification numbers for each and the bacterial isolates they were identified from.

TABLE 15

| | Identity to | | | | | |
|---|---|---|---|---|---|---|
| IPD Name | IPD111Aa | Source | Species | activity | Protein | DNA |
| IPD111Aa | | JH59138-1 | Burkholderia | WCRW | SEQ ID NO: 453 | SEQ ID NO: 629 |
| | | | ambifaria | | | |
| IPD111Ab | 98.2% | SSP672A11-1, SSP674A8-1 | Burkholderia | | SEQ ID NO: 454 | |
| | | | ambifaria | | | |
| IPD111Ac | 98.2% | JH72812-1 | Burkholderia | WCRW | SEQ ID NO: 455 | SEQ ID NO: 630 |
| | | | diffusa | | | |
| IPD111Ad | 97.3% | JH72375-2, JH72383-2 | Burkholderia | WCRW, SBL, | SEQ ID NO: 456 | SEQ ID NO: 631 |
| | | | ambifaria | CEW, FAW | | |
| IPD111Ba | 84.5% | WP_069242065 | Burkholderia fatens | | SEQ ID NO: 457 | |
| IPD111Ca | 73.8% | WP_47904279 | | | SEQ ID NO: 458 | |
| IPD111Cb | 74.4% | WP_065504152 | Burkholderia stabilis | | SEQ ID NO: 459 | |
| IPD111Da | 65.8% | WP_057929659 | Burkholderia ambifaria | | SEQ ID NO: 460 | |
| IPD111Db | 64.9% | WP_057926891 | Burkholderia ambifaria | | SEQ ID NO: 461 | |
| IPD111Fa | 48.4% | JH33490-1, JH44778-1 | Burkholderia ambifaria | WCRW | SEQ ID NO: 462 | SEQ ID NO: 632 |
| IPD111Fb | 47.9% | SSP652C4-1 | Burkholderia sp. | | SEQ ID NO: 463 | |
| IPD111Fc | 49.6% | WP_06759785 | | | SEQ ID NO: 464 | |
| IPD111Fd | 48.7% | JH7091-2; JH47108-1; | Burkholderia ambifaria | | SEQ ID NO: 465 | |
| | | JH51223-1 | | | | |
| IPD111Fe | 49% | JH51372-1; SSP283G7-1 | Burkholderia fata | | SEQ ID NO: 466 | |
| IPD111Ff | 49.6% | JH31254-2 | Burkholderia cenocepacia | | SEQ ID NO: 467 | |
| IPD111Fg | 49.6% | JH11173-1 | Burkholderia ambifaria | | SEQ ID NO: 468 | |
| IPD111Fh | 49.3% | JH91810-2 | Burkholderia ambifaria | | SEQ ID NO: 469 | |
| IPD111Fi | 48.4% | SSP657E4-1 | Burkholderia ambifaria | | SEQ ID NO: 470 | |
| IPD111Fj | 48.8% | JH58609-1 | Burkholderia ambifaria | | SEQ ID NO: 471 | |
| IPD111Fk | 48.4% | SSP652A2-1 | Burkholderia ambifaria | | SEQ ID NO: 472 | |
| IPD111Fl | 43.8% | SSP283D11-1 | Burkholderia ambifaria | | SEQ ID NO: 473 | |
| IPD111Fm | 49.6% | D6270030 | Taxon P0024 pooled | | SEQ ID NO: 474 | |
| | | | samples | | | |
| IPD111Fn | 48.7% | D6270042 | Taxon P0067 pooled | | SEQ ID NO: 475 | |
| | | | samples | | | |
| IPD111Fo | 48.5% | D6270045 | Taxon P0115 pooled | | SEQ ID NO: 476 | |
| | | | samples | | | |
| IPD111Fp | 49.6% | WP_059523551_lipase | Burkholderia cepacia | | SEQ ID NO: 477 | |
| IPD111Fq | 40% | Taxon Pooled | Taxon D684 pooled | | SEQ ID NO: 478 | |
| | | P0038_D6840007_NODE_500_415 | sample | | | |
| IPD111Fr | 48% | P0123_D6840018_NODE_1450_17 | Taxon D684 pooled | | SEQ ID NO: 479 | |
| | | | sample | | | |
| IPD111Fs | 49.6% | WP_059769828_lipase | Burkholderia territorii | | SEQ ID NO: 480 | |
| IPD111Fl | 49.6% | WP_059506011_lipase | Burkholderia territorii | | SEQ ID NO: 481 | |
| IPD111Fu | 49.6% | WP_060136307_lipase | Burkholderia territorii | | SEQ ID NO: 482 | |
| IPD111Fv | 49.6% | WP_060257678_lipase | Burkholderia territorii | | SEQ ID NO: 483 | |
| IPD111Fw | 49.6% | WP_060295530_lipase | Burkholderia territorii | | SEQ ID NO: 484 | |
| IPD111Fx | 49.6% | WP_059545996_lipase | Burkholderia territorii | | SEQ ID NO: 485 | |
| IPD111Fy | 49.3% | WP_060348612_lipase | Burkholderia territorii | | SEQ ID NO: 486 | |
| IPD111Fz | 49.3% | WP_060103013_lipase | Burkholderia territorii | | SEQ ID NO: 487 | |
| IPD111Faa | 49% | WP_059870323_lipase | Burkholderia territorii | | SEQ ID NO: 488 | |
| IPD111Fab | 41.7% | SSP965C9-1 | Pseudomonas | WCRW | SEQ ID NO: 489 | SEQ ID NO: 633 |
| | | | chlororaphis | | | |
| IPD111Fac | 49.3% | WP_059702086 | | | SEQ ID NO: 490 | |
| IPD111Fad | 49.3% | WP_059691143 | | | SEQ ID NO: 491 | |
| IPD111Fae | 49% | WP_059951984 | | | SEQ ID NO: 492 | |
| IPD111Faf | 49.3% | WP_059449404 | | | SEQ ID NO: 493 | |
| IPD111Fah | 49% | WP_060330215 | | | SEQ ID NO: 494 | |
| IPD111Fai | 49% | WP_060121537 | | | SEQ ID NO: 495 | |
| IPD111Faj | 44.4% | AMP40097_lipase | Ralstonia solanacearum | | SEQ ID NO: 496 | |
| IPD111Fak | 40.2% | JH22700-1 | | | SEQ ID NO: 497 | |
| IPD111Fal | 40.2% | JH93224-1 | | | SEQ ID NO: 498 | |
| IPD111Fam | 47.9% | JH51223-1 | | | SEQ ID NO: 499 | |
| IPD111Fan | 41.1% | SDS88364_Lipase | Pseudomonas | | SEQ ID NO: 500 | |
| | | | chloraphis | | | |
| IPD111Fao | 40.2% | BAV76361_lipase | Pseudomonas | | SEQ ID NO: 501 | |
| | | | chloraphis | | | |
| IPD111Ga | 38.5% | P0391 | Taxon pooled samples | | SEQ ID NO: 502 | |
| IPD111Gb | 36.5% | P0407 | Taxon pooled samples | | SEQ ID NO: 503 | |
| IPD111Gc | 39.4% | internal pooled YJP-8 | | | SEQ ID NO: 504 | |
| IPD111Gd | 39.4% | internal pooled YJP-7 | | | SEQ ID NO: 505 | |
| IPD111Ge | 36.3% | P0391 | Taxon pooled samples | | SEQ ID NO: 506 | |
| IPD111Gf | 37.4% | XM27_pooled_NODE_8699_33 | | | SEQ ID NO: 507 | |
| IPD111Gg | 39.9% | XM31_pooled_NODE_68_2723 | | | SEQ ID NO: 508 | |
| IPD111Gh | 38.5% | XM27_pooled_NODE_11503_14 | | | SEQ ID NO: 509 | |
| IPD111Gi | 39.4% | WP_062922774 | | | SEQ ID NO: 510 | |
| IPD111Gj | 39.4% | PMC3632G10-1 | | | SEQ ID NO: 511 | |
| IPD111Gk | 39.9% | PMCJ329E8-1 | | | SEQ ID NO: 512 | |
| IPD111Gl | 39.9% | XM27_pooled_NODE_152_1222 | | | SEQ ID NO: 513 | |
| IPD111Gm | 38.8% | PMC3093AB-1 | Pseudomonas baetica | | SEQ ID NO: 514 | |
| IPD111Gn | 39.9% | JH20450-1 | | | SEQ ID NO: 515 | |
| IPD111Go | 39.7% | JH52581-2 | | | SEQ ID NO: 516 | |
| IPD111Gp | 39.7% | JH19881-4 | | | SEQ ID NO: 517 | |
| IPD111Gq | 39.7% | JH20401-2 | | | SEQ ID NO: 518 | |
| IPD111Gr | 39.7% | JH19896-4 | | | SEQ ID NO: 519 | |
| IPD111Gs | 39.9% | JH25061-1 | | | SEQ ID NO: 520 | |
| IPD111Gt | 39.9% | JH106357-1 | | | SEQ ID NO: 521 | |
| IPD111Gu | 37.1% | JH18110-4 | | | SEQ ID NO: 522 | |
| IPD111Gv | 39% | SDU67175_lipase | Pseudomonas | | SEQ ID NO: 523 | |
| | | | mediterranea | | | |
| IPD111Gw | 35.5% | SDU97137_lipase | Pseudomonas corrugata | | SEQ ID NO: 524 | |
| IPD111Gx | 31.2% | WP_071810643_lipase | Burkholderia | | SEQ ID NO: 525 | |
| | | | pseudomallei | | | |
| IPD111Gy | 39.9% | PMC_34221C2-1 | Pseudomonas chlororaphis | | SEQ ID NO: 526 | SEQ ID NO: 634 |
| IPD111Gz | 37.4% | WP_072394639_lipase | Pseudomanas sp. | | SEQ ID NO: 527 | |
| IPD111Gaa | 31.5% | WP_071893161 | Burkholderia | | SEQ ID NO: 528 | |
| | | | pseudomallei | | | |

Table 16 shows the IPD112Aa polyptide homologs identified, sequence identification numbers for each and the bacterial isolates they were identification.

TABLE 16

| | Identity to | | | | | |
|---|---|---|---|---|---|---|
| IPD Name | IPD112Aa | Source | Species | activity | | |
| IPD112Aa | | SSP640H4-1, SSP640H7-1 | Pseudomonas vranovensis | WCRW | SEQ ID NO: 529 | SEQ ID NO: 635 |
| IPD112Ab | 90% | SSP1049G2-1 | Pseudomonas | | SEQ ID NO: 530 | |
| | | | mosselii | | | |
| IPD112Ea | 54.7% | JH91108-2; SSP519B5b; | Pseudomonas vranovensis | WCRW | SEQ ID NO: 531 | SEQ ID NO: 636 |
| | | JH91611-1; gi771573074 | | | | |
| IPD112Eb | 52.5% | JH31230-2; SSP473A12-1 | Pseudomonas poae | WCRW | SEQ ID NO: 532 | SEQ ID NO: 637 |
| IPD112Ec | 50.2% | parial_WP_048401789 | | | SEQ ID NO: 533 | |
| IPD112Fa | 46.5% | JH68858-1; 15AKG2; | Pseudomonas | WCRW, | SEQ ID NO: 534 | SEQ ID NO: 638 |
| | | JH67425-2; JH67950-2 | protegens | SGSB | | |
| IPD112Fb | 49.7% | NCBI gi860274938 | | | SEQ ID NO: 535 | |
| IPD112Fc | 49.7% | WP_059763362 | Pseudomonas | | SEQ ID NO: 536 | |
| IPD112Fd | 46.5% | PMC353B5-1 | | | SEQ ID NO: 537 | |
| IPD112Ga | 39.5% | NCBI gi902539997 | | | SEQ ID NO: 538 | |
| IPD112Gb | 38.9% | NCBI gi748796500 | | | SEQ ID NO: 539 | |
| IPD112Gc | 38.9% | NCBI gi759783199 | | | SEQ ID NO: 540 | |
| IPD112Gd | 38.9% | NCBI gi815717717 | | | SEQ ID NO: 541 | |
| IPD112Ge | 38.6% | NCBI gi764039999 | | | SEQ ID NO: 542 | |
| IPD112Gf | 37.3% | NCBI gi496088163 | | | SEQ ID NO: 543 | |
| IPD112Gg | 37.9% | WP_061060315 | | | SEQ ID NO: 544 | |
| IPD112Gh | 38.9% | A0A1C6Z1H7 | Hafnia alvei | | SEQ ID NO: 545 | |

### Example 8

### Gene Subcloning and E. coli Expression

The target genes encoding the insecticidal proteins were first amplified by PCR using their genomic DNA as templates. The PCR primers were designed based on the 5′ end and 3′ end sequences of the gene either with appropriate restriction site incorporated or with added sequences overlapping with the 5′ and 3′ ends of the linearized E. coli expression vector. With restriction enzyme digestion and ligation or homolog recombination based cloning, the PCR products were cloned into selected E. coli expression vectors, i.e. pCOLD™ 1, 3, pET16, 24, 28 for N-, C-His tag and no tag expression. In some cases, pMAL™ vector was used for MBP fusion expression. In case of co-expression of two proteins for binary toxins, their native operon sequences were also cloned into one of the E. coli vectors. The proteins were expressed in BL21(DE3), C41 or SHuffle® E. coli hosts cells with 1 mM IPTG overnight induction at 16° C.

The recombinant protein was extracted from E. coli culture after induction. Cell clear lysates or purified proteins were assayed against WCRW as described in Exemple 1.

The purify recombinant proteins were tested on each insect target with dilution series and the minimal inhibitory concentrations were calculated (Table 17 and Table 16).

Table 17. IPD proteins and their minimal inhibitory concentrations on insect targets based on incorporation in artificial diet bioassays.

TABLE 17

| Protein | WCRW | | CEW | ECB | FAW | SBL | VBC |
|---|---|---|---|---|---|---|---|
| IPD092-1Aa/IPD092-2Aa | ~25 ppm/~35 ppm | | n.t.* | | | n.t. | n.t. |
| IPD097Aa | ~600 | ppm | n.t. | | | n.t. | n.t. |
| IPD100-1Aa/IPD100-2Aa | ~1 ppm/~2 ppm | | n.t. | n.t. | n.t. | n.t. | n.t. |
| IPD105Aa | 2667 | ppm | n.t. | n.t. | n.t. | n.t. | n.t. |
| IPD107Aa | 1100 | ppm | n.t. | n.t. | n.t. | n.t. | n.t. |
| IPD112Aa | 160 | ppm | >1250 ppm | >1250 ppm | >1250 ppm | >1250 ppm | n.t. |

*not tested

Table 18. IPD099 and IPD111 proteins and their minimal inhibitory concentrations on insect targets based on overlay artificial diet bioassays.

TABLE 18

| Protein | WCRW | | CEW | ECB | FAW | SBL | VBC |
|---|---|---|---|---|---|---|---|
| IPD099-1Aa/ | ~15/15/15 | μg/cm² | >15/15/15 | >15/15/15 | >15/15/15 | >15/15/15 | >15/15/15 |
| IPD099-2Aa/ | | | μg/cm² | μg/cm² | μg/cm² | μg/cm² | μg/cm² |
| IPD099-3Aa | | | | | | | |
| IPD099-2Aa | ~30 | μg/cm² | n.t.* | n.t. | n.t. | n.t. | n.t. |
| IPD111Aa | 74 | μg/cm² | n.t. | n.t. | n.t. | n.t. | n.t. |

*not tested

Table 19. IPD095 and IPD106 proteins active against WCRW when assayed as unpurified protein in crude E. coli lysates after expression in E. coli.

TABLE 19

| Protein | WCRW |
|---|---|
| IPD095-1Aa/IPD095-2Aa | active |
| IPD106-1Aa/IPD106-2Aa | active |

### Example 9

### Agrobacterium-Mediated Stable Transformation of Maize

For Agrobacterium-mediated maize transformation of insecticidal polypeptides, the method of Zhao is employed (U.S. Pat. No. 5,981). Briefly, immature embryos are isolated from maize and the embryos contacted with an Agrobacterium Suspension, where the bacteria were capable of transferring a polynucleotide encoding an insecticidal polypeptide of the disclosure to at least one cell of at least one of the immature embryos (step 1: the infection step). In this step the immature embryos are immersed in an Agrobacterium suspension for the initiation of inoculation. The embryos are co-cultured for a time with the Agrobacterium (step 2: the co-cultivation step). The immature embryos are cultured on solid medium with antibiotic, but without a selecting agent, for Agrobacterium elimination and for a resting phase for the infected cells. Next, inoculated embryos are cultured on medium containing a selective agent and growing transformed callus is recovered (step 4: the selection step). The immature embryos are cultured on solid medium with a selective agent resulting in the selective growth of transformed cells. The callus is then regenerated into plants (step 5: the regeneration step), and calli grown on selective medium are cultured on solid medium to regenerate the plants.

For detection of the insecticidal polypeptide in leaf tissue 4 lyophilized leaf punches/sample are pulverized and resuspended in 100 μL PBS buffer containing 0.1% TWEEN™ 20 (PBST), 1% beta-mercaoptoethanol containing 1 tablet/7 mL complete Mini proteinase inhibitor (Roche 1183615301). The suspension is sonicated for 2 min and then centrifuged at 4° C., 20,000 g for 15 min. To a supernatant aliquot ⅓ volume of 3× NuPAGE® LDS Sample Buffer (Invitrogen™ (CA, USA), 1% B-ME containing 1 tablet/7 mL complete Mini proteinase inhibitor was added. The reaction is heated at 80° C. for 10 min and then centrifuged. A supernatant sample is loaded on 4-12% Bis-Tris Midi gels with MES running buffer as per manufacturer's (Invitrogen™) instructions and transferred onto a nitrocellulose membrane using an iBlot® apparatus (Invitrogen™). The nitrocellulose membrane is incubated in PBST containing 5% skim milk powder for 2 hours before overnight incubation in affinity-purified rabbit anti-insecticidal polypeptide in PBST overnight. The membrane is rinsed three times with PBST and then incubated in PBST for 15 min and then two times 5 min before incubating for 2 hours in PBST with goat anti-rabbit-HRP for 3 hours. The detected proteins are visualized using ECL Western Blotting Reagents (GE Healthcare cat #RPN2106) and Kodak® Biomax® MR film. For detection of the insecticidal protein in roots the roots are lyophilized and 2 mg powder per sample is suspended in LDS, 1% beta-mercaptoethanol containing 1 tablet/7 mL Complete Mini proteinase inhibitor is added. The reaction is heated at 80° C. for 10 min and then centrifuged at 4° C., 20,000 g for 15 min. A supernatant sample is loaded on 4-12% Bis-Tris Midi gels with MES running buffer as per manufacturer's (Invitrogen™) instructions and transferred onto a nitrocellulose membrane using an iBlot® apparatus (Invitrogen™). The nitrocellulose membrane is incubated in PBST containing 5% skim milk powder for 2 hours before overnight incubation in affinity-purified polyclonal rabbit anti-insecticidal antibody in PBST overnight. The membrane is rinsed three times with PBST and then incubated in PBST for 15 min and then two times 5 min before incubating for 2 hours in PBST with goat anti-rabbit-HRP for 3 hrs. The antibody bound insecticidal proteins are detected using ECL™ Western Blotting Reagents (GE Healthcare cat #RPN2106) and Kodak® Biomax® MR film.

Transgenic maize plants positive for expression of the insecticidal proteins are tested for pesticidal activity using standard bioassays. Such methods include, for example, root excision bioassays and whole plant bioassays. See, e.g., US Patent Application Publication Number US 2003/0120054.

### Example 10

### Expression Vector Constructs for Expression of Insecticidal Polypeptides in Plants

The plant expression vectors, can be constructed to include a transgene cassette containing the coding sequence pf the insecticidal polypeptide, under control of the Mirabilis Mosaic Virus (MMV) promoter [Dey N and Maiti I B, 1999, Plant Mol. Biol. 40(5):771-82] in combination with an enhancer element. These constructs can be used to generate transgenic maize events to test for efficacy against corn rootworm provided by expression of the insecticidal polypeptide of the disclosure.

T0 greenhouse efficacy of the events can be measured by root protection from Western corn rootworm. Root protection is measured according to the number of nodes of roots injured (CRWNIS=corn rootworm node injury score) using the method developed by Oleson, et al. (2005) [J. Econ Entomol. 98(1):1-8]. The root injury score is measured from “0” to “3” with “0” indicating no visible root injury, “1” indicating 1 node of root damage, “2” indicating 2 nodes or root damage, and “3” indicating a maximum score of 3 nodes of root damage. Intermediate scores (e.g. 1.5) indicate additional fractions of nodes of damage (e.g. one and a half nodes injured).

The above description of various illustrated embodiments of the disclosure is not intended to be exhaustive or to limit the scope to the precise form disclosed. While specific embodiments of and examples are described herein for illustrative purposes, various equivalent modifications are possible within the scope of the disclosure, as those skilled in the relevant art will recognize. The teachings provided herein can be applied to other purposes, other than the examples described above. Numerous modifications and variations are possible in light of the above teachings and, therefore, are within the scope of the appended claims.

These and other changes may be made considering the above detailed description. In general, in the following claims, the terms used should not be construed to limit the scope to the specific embodiments disclosed in the specification and the claims.

The entire disclosure of each document cited (including patents, patent applications, journal articles, abstracts, manuals, books or other disclosures) in the Background, Detailed Description, and Examples is herein incorporated by reference in their entireties.

Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight; temperature is in degrees centigrade; and pressure is at or near atmospheric.

That which is claimed is:

1. A recombinant insecticidal polypeptide selected from:
a) a polypeptide comprising an amino acid sequence having at least 80% sequence identity to SEQ ID NO: 1 or fragments thereof having insecticidal activity;
b) a polypeptide comprising an amino acid sequence having at least 80% sequence identity to SEQ ID NO: 2 or fragments thereof having insecticidal activity;
c) a polypeptide comprising an amino acid sequence having at least 80% sequence identity to SEQ ID NO: 27 or fragments thereof having insecticidal activity;
d) a polypeptide comprising an amino acid sequence having at least 80% sequence identity to SEQ ID NO: 28 or fragments thereof having insecticidal activity;
e) a polypeptide comprising an amino acid sequence having at least 80% sequence identity to SEQ ID NO: 121 or fragments thereof having insecticidal activity;
f) a polypeptide comprising an amino acid sequence having at least 80% sequence identity to SEQ ID NO: 136 or fragments thereof having insecticidal activity;
g) a polypeptide comprising an amino acid sequence having at least 80% sequence identity to SEQ ID NO: 137 or fragments thereof having insecticidal activity;
h) a polypeptide comprising an amino acid sequence having at least 80% sequence identity to SEQ ID NO: 138 or fragments thereof having insecticidal activity;
i) a polypeptide comprising an amino acid sequence having at least 80% sequence identity to SEQ ID NO: 332 or fragments thereof having insecticidal activity;
j) a polypeptide comprising an amino acid sequence having at least 80% sequence identity to SEQ ID NO: 333 or fragments thereof having insecticidal activity;
k) polypeptide comprising an amino acid sequence having at least 80% sequence identity to SEQ ID NO: 350 or fragments thereof having insecticidal activity;
l) a polypeptide comprising an amino acid sequence having at least 80% sequence identity to SEQ ID NO: 377 or fragments thereof having insecticidal activity;
m) a polypeptide comprising an amino acid sequence having at least 80% sequence identity to SEQ ID NO: 453 or fragments thereof having insecticidal activity; and
n) a polypeptide comprising an amino acid sequence having at least 80% sequence identity to SEQ ID NO: 529 or fragments thereof having insecticidal activity
wherein the insecticidal polypeptide is operably joined to a heteroloqous signal sequence or a transit sequence.

2. The recombinant insecticidal polypeptide of claim 1, wherein the insecticidal polypeptide is selected from:
a) a polypeptide comprising an amino acid sequence having at least 95% sequence identity to SEQ ID NO: 1 or fragments thereof having insecticidal activity;
b) a polypeptide comprising an amino acid sequence having at least 95% sequence identity to SEQ ID NO: 2 or fragments thereof having insecticidal activity;
c) a polypeptide comprising an amino acid sequence having at least 95% sequence identity to SEQ ID NO: 27 or fragments thereof having insecticidal activity;
d) a polypeptide comprising an amino acid sequence having at least 95% sequence identity to SEQ ID NO: 28 or fragments thereof having insecticidal activity;
e) a polypeptide comprising an amino acid sequence having at least 95% sequence identity to SEQ ID NO: 121 or fragments thereof having insecticidal activity;
f) a polypeptide comprising an amino acid sequence having at least 95% sequence identity to SEQ ID NO: 136 or fragments thereof having insecticidal activity;
g) a polypeptide comprising an amino acid sequence having at least 95% sequence identity to SEQ ID NO: 137 or fragments thereof having insecticidal activity;
h) a polypeptide comprising an amino acid sequence having at least 95% sequence identity to SEQ ID NO: 138 or fragments thereof having insecticidal activity;
i) a polypeptide comprising an amino acid sequence having at least 95% sequence identity to SEQ ID NO: 332 or fragments thereof having insecticidal activity;
j) a polypeptide comprising an amino acid sequence having at least 95% sequence identity to SEQ ID NO: 333 or fragments thereof having insecticidal activity;
k) a polypeptide comprising an amino acid sequence having at least 95% sequence identity to SEQ ID NO: 350 or fragments thereof having insecticidal activity;
l) a polypeptide comprising an amino acid sequence having at least 95% sequence identity to SEQ ID NO: 377 or fragments thereof having insecticidal activity;
m) a polypeptide comprising an amino acid sequence having at least 95% sequence identity to SEQ ID NO: 453 or fragments thereof having insecticidal activity; and
n) a polypeptide comprising an amino acid sequence having at least 95% sequence identity to SEQ ID NO: 529 or fragments thereof having insecticidal activity;
wherein the insecticidal polypeptide is operably ioined to a heteroloqous signal sequence or a transit sequence.

3. (canceled)

4. A composition comprising at least one recombinant insecticidal polypeptide of claim 1.

5. The composition of claim 4, wherein the composition comprises: a polypeptide comprising an amino acid sequence having at least 80% sequence identity to SEQ ID NO: 1 or fragments thereof having insecticidal activity; and a polypeptide comprising an amino acid sequence having at least 80% sequence identity to SEQ ID NO: 2 or fragments thereof having insecticidal activity.

6. The composition of claim 4, wherein the composition comprises: a polypeptide comprising an amino acid sequence having at least 80% sequence identity to SEQ ID NO: 27 or fragments thereof having insecticidal activity; and a polypeptide comprising an amino acid sequence having at least 80% sequence identity to SEQ ID NO: 28 or fragments thereof having insecticidal activity.

7. The composition of claim 4, wherein the composition comprises: a polypeptide comprising an amino acid sequence having at least 80% sequence identity to SEQ ID NO: 136 or fragments thereof having insecticidal activity; a polypeptide comprising an amino acid sequence having at least 80% sequence identity to SEQ ID NO: 137 or fragments thereof having insecticidal activity; and a polypeptide comprising an amino acid sequence having at least 80% sequence identity to SEQ ID NO: 138 or fragments thereof having insecticidal activity.

8. The composition of claim 4, wherein the composition comprises: a polypeptide comprising an amino acid sequence having at least 80% sequence identity to SEQ ID NO: 332 or fragments thereof having insecticidal activity; and a polypeptide comprising an amino acid sequence having at least 80% sequence identity to SEQ ID NO: 333 or fragments thereof having insecticidal activity.

9. A recombinant polynucleotide encoding the insecticidal polypeptide of claim 1.

10. The recombinant polynucleotide of claim 9, wherein the polynucleotide has codons optimized for expression in an agriculturally important crop.

11. A DNA construct comprising the recombinant polynucleotide of claim 9 operably linked to a heterologous regulatory element.

12. A transgenic plant or plant cell comprising the polynucleotide of claim 9.

13. A transgenic plant comprising the DNA construct of claim 10.

14. A method of inhibiting growth or killing an insect pest or pest population, comprising contacting the insect pest with the insecticidal polypeptide of claim 1.

15. A method of inhibiting growth or killing an insect pest or pest population, comprising contacting the insect pest with the composition of claim 4.

16. A method of inhibiting growth or killing an insect pest or pest population comprising expressing in a plant the polynucleotide of claim 9.

17. The method of claim 14, wherein the insect pest or pest population is resistant to at least one Cry insecticidal protein.

18. A transformed prokaryotic cell comprising the polynucleotide of claim 9.
